(19) United States
(12) Patent Application Publication
(10) Pub. No.: US 2009/0163454 A1
(43) Pub. Date: Jun. 25, 2009

(54) Methods of Step-Down Hormone Treatment

(75) Inventors: Howard Hait, Wilmington, DE (US); Kathleen Reape, Newtown Square, PA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Duramed Pharmaceuticals, Inc., Montvale, NJ (US)

(21) Appl. No.: 12/341,789

(22) Filed: Dec. 22, 2008

Related U.S. Application Data
(63) Continuation-in-part of application No. 10/837,268, filed on May 3, 2004.
Application No. 10/837,268 is a continuation-in-part of application No. 10/892,404, filed on Jul. 16, 2004.
(60) Provisional application No. 60/467,172, filed on May 2, 2003.
(60) Provisional application No. 60/524,081, filed on Nov. 24, 2003.
(60) Provisional application No. 60/487,257, filed on Jul. 16, 2003.

(51) Int. Cl. A61K 31/56; A61P 15/18

(52) U.S. Cl. 514/170

(57) ABSTRACT

The present invention relates to methods of step-down hormone treatment in which an estrogen and progestin combination regimen is administered for one or more cycles prior to administration of a lower dose hormone regimen containing a lower daily dosage amount of estrogen and/or progestin.

### CROSS-REFERENCE TO RELATED APPLICATIONS

This application is a continuation-in-part of U.S. application Ser. No. 10/837,268, filed May 3, 2004, which claims the benefit, under 35 U.S.C. § 119(e), of U.S. Provisional Application No. 60/467,172, filed on May 2, 2003, and U.S. Provisional Application No. 60/524,081, filed on Nov. 24, 2003. This application is also a continuation-in-part of U.S. application Ser. No. 10/892,404, filed Jul. 16, 2004, which claims the benefit, under 35 U.S.C. § 119(e), of U.S. Provisional Application No. 60/487,257, filed on Jul. 16, 2003. The contents of each of these applications are herein incorporated by reference in their entireties.

### BACKGROUND OF THE INVENTION

1. Field of the Invention

The present invention relates to methods of step-down hormone treatment in which an estrogen and progestin combination regimen is administered for one or more cycles prior to administration of a lower dose estrogen and/or progestin regimen.

2. Related Art

The ovarian/menstrual cycle is a complex event characterized by an estrogen rich follicular phase and, after ovulation, a progesterone rich luteal phase. Each phase has a duration of approximately 14 days resulting in an intermenstrual interval of about 28 days. The endometrial tissue responds to the changes in the hormonal milieu.

The follicular and luteal phases correspond to changes occurring in the ovary. These phases may also be described as proliferative or secretory, corresponding to changes observed in the uterine endometrium. Variations in the length of the cycle are usually due to alterations in the follicular phase, because the luteal phase length remains relatively constant at about 12 days to about 16 days.

During the follicular phase, several primary follicles are recruited for further growth and development. Granulosa cells in primary follicles possess follicle stimulating hormone (FSH) and estradiol receptors. Upon FSH stimulation, granulosa cells produce aromatase. This enzyme converts the androgens androstenedione and testosterone, made in response to luteinizing hormone (LH) by thecal cells, to estrone and estradiol, respectively. Granulosa cells respond to estradiol by undergoing mitosis to increase the number of granulosa cells and estradiol production. By day 7 of the cycle, one enlarging primary follicle is selected by unknown processes to be the follicle that will release the oocyte at ovulation.

The midcycle rise in plasma estradiol stimulates the large midcycle LH surge. This midcycle LH surge triggers resumption of meiosis within the oocyte and luteinization of the granulosa cells within the preovulatory follicle. Immediately before ovulation, the outer follicular wall begins to dissolve and an oocyte is released approximately 24 to 36 hours from the onset of the LH surge.

After ovulation, granulosa cells and the surrounding thecal cells enlarge, accumulate lipid, and become transformed into lutein cells. This begins the luteal phase of the menstrual cycle. These cells form a new vascularized structure called the corpus luteum, which secretes estradiol and progesterone. LH maintains the corpus luteum during the luteal phase and, acting via the adenyl cyclase system, stimulates progesterone production. If pregnancy does not occur, lutein cells degenerate, and diminished hormone secretion precedes menstruation. Menstruation is immediately followed by the onset of another menstrual cycle.

The onset of menstruation is generally considered to be the beginning of a new menstrual cycle and is generally counted as Day 1. During a span of about 5 to 7 days, the superficial layers of the endometrium, which grew and developed during the antecedent ovarian/menstrual cycle, are sloughed because demise of the corpus luteum in the non-fertile menstrual cycle is associated with a loss of progesterone secretion.

Ovarian follicular maturation occurs progressively resulting in a rise in the circulating levels of estrogen, which in turn leads to new endometrial proliferation.

The dominant ovarian follicle undergoes ovulation at mid-cycle, generally between menstrual cycle days 12 to 16 and is converted from a predominantly estrogen source to a predominantly progesterone source (the corpus luteum). The increasing level of progesterone in the blood converts the proliferative endometrium to a secretory phase in which the tissue proliferation has promptly abated, leading to the formation of endometrial glands or organs. When the ovulated oocyte is viably fertilized and continues its progressive embryonic cleavage, the secretory endometrium and the conceptus can interact to bring about implantation (nidation), beginning about 6 to 8 days after fertilization.

If an ongoing pregnancy is to be established via implantation, the embryo will attach and burrow into the secretory endometrium and begin to produce human chorionic gonadotropin (HCG). The HCG in turn stimulates extended corpus luteum function, i.e., the progesterone production remains elevated, and menses does not occur in the fertile menstrual cycle. Pregnancy is then established.

In the non-fertile menstrual cycle, the waning level of progesterone in the blood causes the endometrial tissue to be sloughed. This starts a subsequent menstrual cycle.

Because endometrial proliferation serves to prepare the uterus for an impending pregnancy, manipulation of hormones and of the uterine environment can provide contraception. For example, estrogens are known to decrease follicle stimulating hormone secretion by feedback inhibition. Under certain circumstances, estrogens can also inhibit luteinizing hormone secretion, once again by negative feedback. Under normal circumstances, the spike of circulating estrogen found just prior to ovulation induces the surge of gonadotropic hormones that occurs just prior to and resulting in ovulation. High doses of estrogen immediately post-coitally also can prevent conception probably due to interference with implantation.

Progestins can also provide contraception. Endogenous progesterone after estrogen is responsible for the progestational changes of the endometrium and the cyclic changes of cells and tissue in the cervix and the vagina. Administration of progestin makes the cervical mucus thick, tenacious and cellular, which is believed to impede spermatozoal transport. Administration of progestin also inhibits luteinizing hormone secretion and blocks ovulation in humans.

The most prevalent form of oral contraception is a pill that combines both an estrogen and a progestin, a so-called combined oral contraceptive preparation. Alternatively, there are contraceptive preparations that comprise progestin only. However, the progestin-only preparations have a more varied spectrum of side effects than do the combined preparations, especially more breakthrough bleeding. As a result, the combined preparations are the preferred oral contraceptives in use today (Sheth et al., Contraception 25:243 (1982)).

Whereas the conventional 21 day pill packs with a 7 day “pill free” or placebo interval worked well when oral contraceptives were of higher dosage, as the doses have come down, for both the estrogen and progestin components, bleeding problems have increased in frequency, especially in the early months of oral contraceptive use, but even persistently so in some patients.

There exists a need for contraceptives that reduce bleeding problems and/or have additional benefits for women.

### SUMMARY OF THE INVENTION

The present invention provides a method of step-down hormone treatment in which an estrogen and progestin combination regimen is administered for one or more cycles prior to administration of a lower dose hormone regimen containing a lower daily dosage amount of estrogen and/or progestin.

The present invention is further directed to a method of reducing unscheduled bleeding or spotting during administration of a hormone regimen in a female, the method comprising:

(a) administering to the female one or more cycles of a run-in regimen, wherein each cycle of the regimen comprises administering to the female a combination of estrogen and progestin for 60 to 110 consecutive days,

wherein the daily amount of estrogen administered for 60 to 110 consecutive days is equivalent to about 5 μg to about 50 μg of ethinyl estradiol,

wherein the daily amount of progestin administered for 60 to 110 consecutive days is equivalent to about 50 μg to about 1.5 mg of levonorgestrel,

wherein the one or more cycles of (a) is followed by

(b) administering to the female one or more cycles of a lower dose hormone regimen,

wherein the daily amount of hormone administered during any period of a cycle comprising administration of hormone is selected from the group consisting of: a daily amount of estrogen that is lower than the daily amount of estrogen administered for 60 to 110 consecutive days in (a); a daily amount of progestin that is lower than the daily amount of progestin administered for 60 to 110 consecutive days in (a); and combinations thereof,

wherein the daily amounts are contraceptive or therapeutic amounts, and

wherein unscheduled bleeding or spotting during administration of the lower dose hormone regimen of (b) is reduced following prior administration of the run-in regimen of (a).

In some aspects, the combination of estrogen and progestin administered during the run-in regimen for 60 to 110 consecutive days is administered for 81 to 110 consecutive days.

In some aspects, the combination of estrogen and progestin administered during the run-in regimen for 60 to 110 consecutive days is administered for 81 to 89 consecutive days.

In some aspects, the combination of estrogen and progestin administered during the run-in regimen for 60 to 110 consecutive days is administered for 84 consecutive days.

In some aspects, each cycle of the run-in regimen comprises:

(i) administering the combination of estrogen and progestin for 60 to 110 consecutive days, followed by

(ii) a period of 2 to 10 consecutive days selected from the group consisting of: a hormone-free period of 2 to 10 consecutive days, wherein neither estrogen nor progestin is administered to the female during the hormone-free period; and a period of 2 to 10 consecutive days wherein estrogen is administered without progestin.

The present invention is further directed to a method of reducing unscheduled bleeding or spotting during administration of a hormone regimen in a female, the method comprising:

(a) administering to the female one or more cycles of a run-in regimen, wherein each cycle of the regimen comprises administering to the female a combination of estrogen and progestin for 21 to 26 consecutive days, wherein the daily amount of estrogen administered for 21 to 26 consecutive days is equivalent to about 5 μg to about 50 μg of ethinyl estradiol, wherein the daily amount of progestin administered for 21 to 26 consecutive days is equivalent to about 50 μg to about 1.5 mg of levonorgestrel, wherein the one or more cycles of (a) is followed by

(b) administering to the female one or more cycles of a lower dose hormone regimen, wherein the daily amount of hormone administered during any period of a cycle comprising administration of hormone is selected from the group consisting of: a daily amount of estrogen that is lower than the daily amount of estrogen administered for 21 to 26 consecutive days in (a); a daily amount of progestin that is lower than the daily amount of progestin administered for 21 to 26 consecutive days in (a); and combinations thereof,

wherein the daily amounts are contraceptive or therapeutic amounts, and

wherein unscheduled bleeding or spotting during administration of the lower dose hormone regimen of (b) is reduced following prior administration of the first hormone regimen of (a).

In some aspects, the combination of estrogen and progestin administered during the run-in regimen for 21 to 26 consecutive days is administered for 21 consecutive days.

In some aspects, the combination of estrogen and progestin administered during the run-in regimen for 21 to 26 consecutive days is administered for 23 to 25 consecutive days.

In some aspects, the combination of estrogen and progestin administered during the run-in regimen for 23 to 25 consecutive days is administered for 25 consecutive days.

In some aspects, each cycle of the run-in regimen administered for 21 to 26 consecutive days comprises:

(i) administering the combination of estrogen and progestin for 21 to 26 consecutive days, followed by

(ii) a period of 2 to 10 consecutive days selected from the group consisting of: a hormone-free period of 2 to 10 consecutive days, wherein neither estrogen nor progestin is administered to the female during the hormone-free period; and a period of 2 to 10 consecutive days wherein estrogen is administered without progestin.

In some aspects, the daily amount of estrogen administered during the run-in regimen of any of the methods above is equivalent to about 10 μg to about 30 μg of ethinyl estradiol, and the daily amount of progestin during the run-in regimen of any of the methods above is equivalent to about 50 μg to about 150 μg of levonorgestrel.

In some aspects, the daily amount of estrogen administered during the run-in regimen of any of the methods above is equivalent to about 30 μg of ethinyl estradiol, and the daily amount of progestin during the run-in regimen of any of the methods above is equivalent to about 150 μg of levonorgestrel.

In some aspects, each cycle of the lower dose hormone regimen in any of the methods above comprises administering to the female a regimen comprising: a combination of estrogen and progestin for 60 to 110 consecutive days; a combination of estrogen and progestin for 21 to 26 consecutive days; an estrogen-only regimen in which estrogen is administered without progestin; or a progestin-only regimen in which progestin is administered without estrogen,

wherein the daily amount of estrogen in the combination of estrogen and progestin or in the estrogen-only regimen is equivalent to about 2 μg to about 50 μg of ethinyl estradiol, and

wherein the daily amount of progestin in the combination of estrogen and progestin or in the progestin-only regimen is equivalent to about 10 μg to about 1.5 mg of levonorgestrel.

In some aspects, each cycle of the lower dose hormone regimen in any of the methods above further comprises a period of 2 to 10 consecutive days selected from the group consisting of: a hormone-free period of 2 to 10 consecutive days wherein neither estrogen nor progestin is administered to the female during the hormone-free period; and a period of 2 to 10 days wherein estrogen is administered without progestin.

In some aspects, the combination of estrogen and progestin administered during the lower dose hormone regimen for 60 to 110 consecutive days in any of the methods above is administered for 81 to 110 consecutive days.

In some aspects, the combination of estrogen and progestin administered during the lower dose hormone regimen for 60 to 110 consecutive days in any of the methods above is administered for 81 to 89 consecutive days.

In some aspects, the combination of estrogen and progestin administered during the lower dose hormone regimen for 60 to 110 consecutive days in any of the methods above is administered for 84 consecutive days.

In some aspects, the combination of estrogen and progestin administered during the lower dose hormone regimen for 21 to 26 consecutive days in any of the methods above is administered for 21 consecutive days.

In some aspects, the combination of estrogen and progestin administered during the lower dose hormone regimen for 21 to 26 consecutive days in any of the methods above is administered for 23 to 25 consecutive days.

In some aspects, the combination of estrogen and progestin administered during the lower dose hormone regimen for 23 to 25 consecutive days in any of the methods above is administered for 25 consecutive days.

In some aspects, the daily amount of estrogen administered during the lower dose hormone regimen of any of the methods above is equivalent to about 10 μg to about 30 μg of ethinyl estradiol, and the daily amount of progestin during the lower dose hormone regimen of any of the methods above is equivalent to about 50 μg to about 150 μg of levonorgestrel.

In some aspects, the daily amount of estrogen administered during the lower dose hormone regimen of any of the methods above is equivalent to about 20 μg of ethinyl estradiol, and the daily amount of progestin during the lower dose hormone regimen of any of the methods above is equivalent to about 100 μg of levonorgestrel.

In some aspects, the period of 2 to 10 consecutive days in any of the run-in or lower dose hormone regimens above is a period of 7 consecutive days.

In some aspects, the period of 2 to 10 consecutive days in any of the run-in or lower dose hormone regimens above is a hormone-free period.

In some aspects, the hormone-free period in any of the run-in or lower dose hormone regimens above is achieved by administering a hormone-free placebo.

In some aspects, the period of 2 to 10 consecutive days in any of the run-in or lower dose hormone regimens above is a period of 2 to 10 consecutive days wherein estrogen is administered without progestin.

In some aspects, the daily amount of estrogen administered during the period of 2 to 10 consecutive days in any of the run-in or lower dose hormone regimens above is equivalent to about 5 μg to about 50 μg of ethinyl estradiol.

In some aspects, the daily amount of estrogen administered during the period of 2 to 10 consecutive days in any of the run-in or lower dose hormone regimens above is equivalent to about 10 μg to about 30 μg of ethinyl estradiol.

In some aspects, the daily amount of estrogen administered during the period of 2 to 10 consecutive days in any of the run-in or lower dose hormone regimens above is equivalent to about 30 μg of ethinyl estradiol.

In some aspects, the daily amount of estrogen administered during the period of 2 to 10 consecutive days in any of the run-in or lower dose hormone regimens above is equivalent to about 10 μg of ethinyl estradiol.

In some aspects, the estrogen administered during any of the run-in or lower dose hormone regimens above is ethinyl estradiol.

In some aspects, the progestin administered during any of the run-in or lower dose hormone regimens above is levonorgestrel.

In some aspects, the progestin administered during any of the run-in or lower dose hormone regimens above is desogestrel.

In some aspects, the estrogen administered during the run-in regimen for 60 to 110 consecutive days is administered for 84 consecutive days and is present in a daily amount of about 30 μg of ethinyl estradiol; the progestin administered during the run-in regimen for 60 to 110 consecutive days is administered for 84 consecutive days and is present in a daily amount of about 150 μg of levonorgestrel; the estrogen administered during the lower dose hormone regimen for 60 to 110 consecutive days is administered for 84 consecutive days and is present in a daily amount of about 20 μg of ethinyl estradiol; the progestin administered during the lower dose hormone regimen for 60 to 110 consecutive days is administered for 84 consecutive days and is present in a daily amount of about 100 μg of levonorgestrel; and the periods of 2 to 10 consecutive days in both the run-in and lower dose hormone regimens are hormone-free periods of 7 consecutive days.

In some aspects, the estrogen administered during the run-in regimen for 60 to 110 consecutive days is administered for 84 consecutive days and is present in a daily amount of about 30 μg of ethinyl estradiol; the progestin administered during the run-in regimen for 60 to 110 consecutive days is administered for 84 consecutive days and is present in a daily amount of about 150 μg of levonorgestrel; the estrogen administered during the lower dose hormone regimen for 60 to 110 consecutive days is administered for 84 consecutive days and is present in a daily amount of about 20 μg of ethinyl estradiol; the progestin administered during the lower dose hormone regimen for 60 to 110 consecutive days is administered for 84 consecutive days and is present in a daily amount of about 100 μg of levonorgestrel; the period of 2 to 10 consecutive days in the run-in regimen is a hormone-free period of 7 consecutive days; and the period of 2 to 10 consecutive days in the lower dose hormone regimen is a period of 7 consecutive days wherein estrogen is administered without progestin in a daily amount of about 10 μg of ethinyl estradiol.

In some aspects, the estrogen administered during the run-in regimen for 60 to 110 consecutive days is administered for 84 consecutive days and is present in a daily amount of about 30 μg of ethinyl estradiol; the progestin administered during the run-in regimen for 60 to 110 consecutive days is administered for 84 consecutive days and is present in a daily amount of about 150 μg of levonorgestrel; the estrogen administered during the lower dose hormone regimen for 60 to 110 consecutive days is administered for 84 consecutive days and is present in a daily amount of about 20 μg of ethinyl estradiol; the progestin administered during the lower dose hormone regimen for 60 to 110 consecutive days is administered for 84 consecutive days and is present in a daily amount of about 100 μg of levonorgestrel; and the periods of 2 to 10 consecutive days in both the run-in and lower dose hormone regimens are periods of 7 consecutive days wherein estrogen is administered without progestin in a daily amount of about 10 μg of ethinyl estradiol.

In some aspects, the estrogen administered during the run-in regimen for 60 to 110 consecutive days is administered for 84 consecutive days and is present in a daily amount of about 30 μg of ethinyl estradiol; the progestin administered during the run-in regimen for 60 to 110 consecutive days is administered for 84 consecutive days and is present in a daily amount of about 150 μg of levonorgestrel; the estrogen administered during the lower dose hormone regimen for 60 to 110 consecutive days is administered for 84 consecutive days and is present in a daily amount of about 20 μg of ethinyl estradiol; the progestin administered during the lower dose hormone regimen for 60 to 110 consecutive days is administered for 84 consecutive days and is present in a daily amount of about 100 μg of levonorgestrel; the period of 2 to 10 consecutive days in the run-in regimen is a period of 7 consecutive days wherein estrogen is administered without progestin in a daily amount of about 10 μg of ethinyl estradiol; and the period of 2 to 10 consecutive days in the lower dose hormone regimen is a hormone-free period of 7 consecutive days.

The present invention is further directed to kits comprising a run-in regimen as described above followed by a lower dose hormone regimen.

In some aspects, the kit comprises:

(a) 60 to 110 tablets for oral administration comprising a combination of estrogen and progestin, wherein:
(1) the estrogen in each of the tablets is present in an amount equivalent to about 5 μg to about 50 μg of ethinyl estradiol; and
(2) the progestin in each of the tablets is present in an amount equivalent to about 0.05 mg to about 1.5 mg of levonorgestrel;

(b) 2 to 10 tablets for oral administration selected from the group consisting of:
(1) 2 to 10 tablets for oral administration consisting essentially of estrogen, wherein the estrogen in each of the tablets is present in an amount equivalent to about 5 μg to about 50 μg ethinyl estradiol; and
(2) 2 to 10 tablets for oral administration consisting of a hormone-free placebo;

wherein the tablets in (a) and (b) are arranged in a fixed sequence that corresponds to the stages of daily administration; and

(c) instructions for administering the tablets of (a) and (b),

wherein the instructions require administration of the tablets of (a) and (b) prior to a lower dose hormone regimen for oral administration, wherein the daily amount of hormone administered during any period of a cycle comprising administration of hormone is selected from the group consisting of: a daily amount of estrogen that is lower than the daily amount of estrogen administered in the 60 to 110 tablets in (a); a daily amount of progestin that is lower than the daily amount of progestin administered in the 60 to 110 tablets in (a); and combinations thereof.

In some aspects, the kit comprises:

(a) 21 to 26 tablets for oral administration comprising a combination of estrogen and progestin, wherein:
(1) the estrogen in each of the tablets is present in an amount equivalent to about 5 μg to about 50 μg of ethinyl estradiol; and
(2) the progestin in each of the tablets is present in an amount equivalent to about 0.05 mg to about 1.5 mg of levonorgestrel;

(b) 2 to 10 tablets for oral administration selected from the group consisting of:
(1) 2 to 10 tablets for oral administration consisting essentially of estrogen, wherein the estrogen in each of the tablets is present in an amount equivalent to about 5 μg to about 50 μg ethinyl estradiol; and
(2) 2 to 10 tablets for oral administration consisting of a hormone-free placebo;

wherein the tablets in (a) and (b) are arranged in a fixed sequence that corresponds to the stages of daily administration; and (c) instructions for administering the tablets of (a) and (b),

wherein the instructions require administration of the tablets of (a) and (b) prior to a lower dose hormone regimen for oral administration, wherein the daily amount of hormone administered during any period of a cycle comprising administration of hormone is selected from the group consisting of: a daily amount of estrogen that is lower than the daily amount of estrogen administered in the 21 to 26 tablets in (a); a daily amount of progestin that is lower than the daily amount of progestin administered in the 21 to 26 tablets in (a); and combinations thereof.

In some aspects, the lower dose hormone regimen is provided in a separate kit. In some aspects, the lower dose hormone regimen is provided in the same kit.

In some aspects, the lower dose hormone regimen is a contraceptive regimen. In some aspects, the lower dose hormone regimen is a hormone replacement therapy regimen.

### BRIEF DESCRIPTION OF THE DRAWINGS

FIG. 1 shows the median number of days of breakthrough bleeding/spotting by cycle in patients receiving the 30 μg ethinyl estradiol (EE)/150 μg levonorgestrel (LNG) 91-day extended cycle regimen in the clinical study described in Example 1.

FIG. 2 shows the percent of patients on the 30 μg EE/150 μg LNG 91-day extended regimen reporting bleeding by study day for the clinical study described in Example 1.

FIG. 3 shows the distribution of bleeding and spotting reported by patients administered the DP3-84/30 regimen during the first clinical study described in Example 7.

FIG. 4 shows the distribution of bleeding and spotting reported by patients administered the DP3-84/10 regimen during the first clinical study described in Example 7.

FIG. 5 shows the distribution of bleeding and spotting reported by patients administered the DP3-84/30 regimen during the second clinical study described in Example 7.

FIG. 6 shows the distribution of bleeding and spotting reported by patients administered the DP3-84/10 regimen during the second clinical study described in Example 7.

FIG. 7 shows the distribution of bleeding and spotting reported by patients administered the DP3-25/30 regimen during the second clinical study described in Example 7.

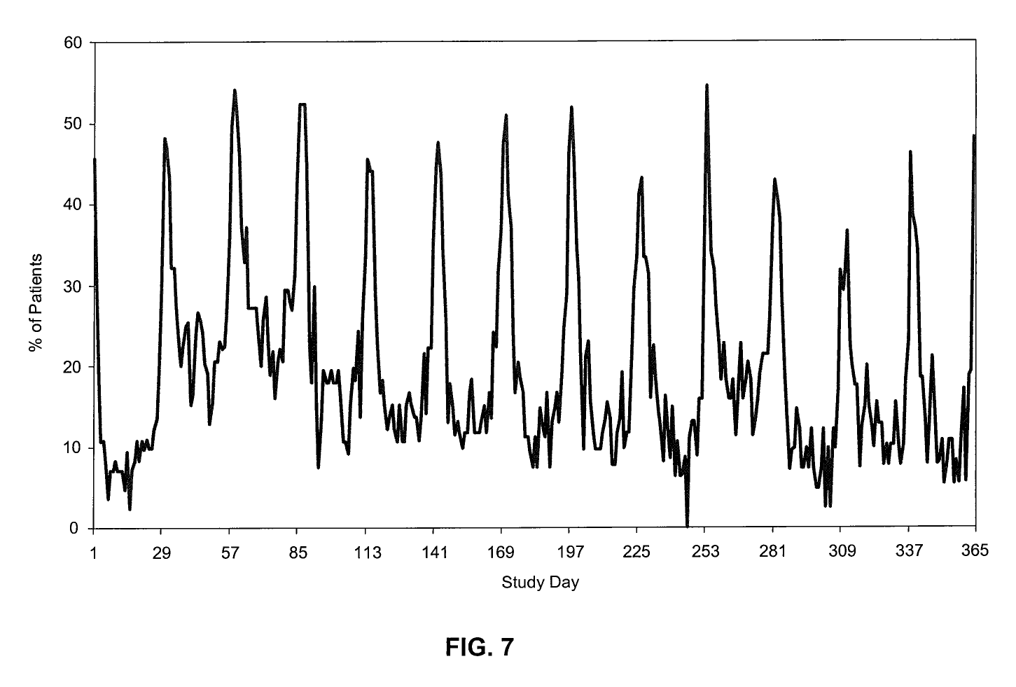

FIG. 8 shows the plasma concentration of Follicle Stimulating Hormone (FSH) in patients during daily administration of levonorgestrel (0.150 mg)/ethinyl estradiol (0.030 mg) tablets for 84 consecutive days, followed by daily administration of ethinyl estradiol (0.030 mg) tablets for 7 days, as described in Example 10. FIG. 8 also shows the plasma concentration of FSH up to about 56 days (Day 147) after completion of administration, during which no hormone was administered to the patients.

FIG. 9 shows the plasma concentration of estradiol in patients during daily administration of levonorgestrel (0.150 mg)/ethinyl estradiol (0.030 mg) tablets for 84 consecutive days, followed by daily administration of ethinyl estradiol (0.030 mg) tablets for 7 days, as described in Example 10. FIG. 9 also shows the plasma concentration of estradiol up to about 56 days (Day 147) after completion of administration, during which no hormone was administered to the patients.

FIG. 10 shows the plasma concentration of Luteinizing Hormone (LH) in patients during daily administration of levonorgestrel (0.150 mg)/ethinyl estradiol (0.030 mg) tablets for 84 consecutive days, followed by daily administration of ethinyl estradiol (0.030 mg) tablets for 7 days, as described in Example 10. FIG. 10 also shows the plasma concentration of LH up to about 56 days (Day 147) after completion of administration, during which no hormone was administered to the patients.

FIG. 11 shows the plasma concentration of free testosterone in patients during daily administration of levonorgestrel (0.150 mg)/ethinyl estradiol (0.030 mg) tablets for 84 consecutive days, followed by daily administration of ethinyl estradiol (0.030 mg) tablets for 7 days, as described in Example 10. FIG. 11 also shows the plasma concentration of free testosterone up to about 56 days (Day 147) after completion of administration, during which no hormone was administered to the patients.

FIG. 12 shows the plasma concentration of total testosterone in patients during daily administration of levonorgestrel (0.150 mg)/ethinyl estradiol (0.030 mg) tablets for 84 consecutive days, followed by daily administration of ethinyl estradiol (0.030 mg) tablets for 7 days, as described in Example 10. FIG. 12 also shows the plasma concentration of total testosterone up to about 56 days (Day 147) after completion of administration, during which no hormone was administered to the patients.

### DETAILED DESCRIPTION OF THE INVENTION

The present invention provides methods of step-down hormone treatment in which one or more cycles of an estrogen and progestin combination regimen is administered to a female prior to administration of a lower dose hormone regimen. The regimen administered prior to the lower dose hormone regimen is alternatively referred to as a “run-in” regimen. Each cycle of the run-in regimen can contain one or more periods, wherein each period can comprise different dosage amounts, different active agents, and/or fewer active agents than those administered in a prior period of the run-in regimen.

The lower daily dosage amount of the estrogen and/or progestin in the lower dose hormone regimen is lower than the daily dosage amount administered during at least one period in each cycle of the run-in regimen. The lower dose hormone regimen can be an extended cycle regimen or can be a conventional or commercially available regimen.

Extended Cycle Regimens

In some aspects of the invention, the run-in regimen or lower dose hormone regimen is an “extended cycle regimen,” i.e., a combined dosage form of estrogen and progestin (or progestogen) that is administered to a female for more than 50 consecutive days, for greater than 50 consecutive days, or for at least 50 consecutive days, in which the daily dosage amount of estrogen can be equivalent to about 5 μg to about 50 μg of ethinyl estradiol and the daily dosage amount of progestin can be equivalent to about 0.01 mg to about 1.5 mg of levonorgestrel.

In some aspects of the invention, the combined dosage form of estrogen and progestin is administered for about 60 to about 110 consecutive days, about 81 to about 110 consecutive days, about 80 to about 90 consecutive days, about 81 to about 89 consecutive days, at least about 81 consecutive days, at least about 84 consecutive days, or about 84 consecutive days.

In one extended cycle regimen of the invention, the period of administration of the combined dosage form of estrogen and progestin is optionally followed by a period of about 2 to about 10 days during which neither estrogen nor progestin is administered (“hormone-free period”). In other aspects of the invention, the hormone-free period is about 5 to about 8 days. In yet other aspects of the invention, the hormone-free period is about 7 days. In some aspects of the invention, the hormone-free period is achieved by administering a hormone-free placebo during that period.

In some embodiments, the extended cycle regimen comprises 84 consecutive days of administration of estrogen and progestin, followed by a hormone-free period of 7 consecutive days.

In another extended cycle regimen of the invention, the period of administration of the combined dosage form of estrogen and progestin (or progestogen) is optionally followed by an estrogen-only period of about 2 to about 10 consecutive days during which estrogen is administered without progestin and in which the daily dosage amount of estrogen can be equivalent to about 5 μg to about 50 μg of ethinyl estradiol (“unopposed estrogen interval”). An extended cycle regimen in which a combined dosage form of estrogen and progestin is followed by an unopposed estrogen interval is also referred to as a “bridged extended cycle regimen.” In some aspects of the invention, an unopposed estrogen interval is about 2 to about 7, about 3 to about 7, about 2 to about 5, about 2 to about 3, or about 5 to about 8 consecutive days. In some aspects of the invention, an unopposed estrogen interval is about 3 consecutive days. In some aspects of the invention, an unopposed estrogen interval is about 7 consecutive days.

In some embodiments, the extended cycle regimen comprises 84 consecutive days of administration of estrogen and progestin, followed by 7 consecutive days of administration of estrogen.

In some aspects of the invention, the daily dosage amount of estrogen in either the extended regimen or an optional unopposed estrogen interval is equivalent to about 5 μg to about 25 μg of ethinyl estradiol. In other aspects of the invention, the daily dose of estrogen in either the extended regimen or an optional unopposed estrogen interval is equivalent to about 25 μg to about 40 μg of ethinyl estradiol. In other aspects of the invention, the daily dose of estrogen in either the extended regimen or an optional unopposed estrogen interval is equivalent to about 10 μg to about 30 μg of ethinyl estradiol. In other aspects of the invention, the daily dose of estrogen in either the extended regimen or an optional unopposed estrogen interval is equivalent to about 20 μg of ethinyl estradiol. In other aspects, the daily dose of estrogen in either the extended regimen or an optional unopposed estrogen interval is equivalent to about 30 μg of ethinyl estradiol. In other aspects, the daily dose of estrogen in the optional unopposed estrogen interval is equivalent to about 5 μg to about 15 μg of ethinyl estradiol. In other aspects, the daily dose of estrogen in the optional unopposed estrogen interval is equivalent to about 10 μg of ethinyl estradiol.

In some aspects of the invention, the daily dosage amount of progestin in the extended regimen is equivalent to about 0.01 mg to about 0.25 mg of levonorgestrel. In other aspects of the invention, the daily dose of progestin in the extended regimen is equivalent to about 0.05 mg to about 0.15 mg of levonorgestrel. In other aspects of the invention, the daily dose of progestin in the extended regimen is equivalent to about 0.1 mg of levonorgestrel. In other aspects, the daily dose of progestin in the extended regimen is equivalent to about 0.15 mg levonorgestrel.

In some aspects of the invention, the extended cycle regimen is optionally administered with an antidepressant. In some aspects of the invention, the antidepressant is administered during a hormone-free period or in combination with estrogen during an unopposed estrogen interval. In other aspects of the invention, the antidepressant is administered continuously throughout the regimen, or, in yet other aspects of the invention, the antidepressant is administered intermittently. For example, in one aspect of the invention, the antidepressant is administered intermittently during the late luteal phase, which is typically one to two weeks before menses. In yet other aspects of the invention, the antidepressant is administered one time during a menstrual cycle, or once weekly.

Methods of Treatment

The regimens disclosed herein can be used as methods of female contraception.

Thus, the invention is directed to methods of providing contraception to a female in need thereof by administering to the female the regimens disclosed herein. The female can be, for example, of childbearing age or peri-menopausal.

However, the regimens are also useful as methods of treating a variety of conditions and disorders in females, including peri-menopausal and menopausal females.

Thus, the regimens can be used as methods of providing contraception to a female for the treatment of a condition or disorder, or as methods of providing contraception and treating a condition or disorder in a female. Such conditions and disorders are described below and include, but are not limited to: breakthrough bleeding; irregular withdrawal bleeding; menstrual bleeding disorders; symptoms associated with an ovarian cyst, uterine leiomyoma (fibroid tumor), and/or Polycystic Ovarian Syndrome; hirsutism; iron deficiency anemia; menstrual disorders; acne; endometriosis; endometrial cancer; ovarian cancer; benign breast disease; infections; ectopic pregnancy; temporomandibular disorder; catamenial symptoms; non-menstrual related headache, nausea, and/or depression; peri-menopausal symptoms; hypoestrogenism; menopausal disorders; and loss of bone density. Menopausal conditions and disorders also include vaginal dryness, pain during intercourse (dyspareunia), increased risk of infections, inability to control urination (incontinence), increased frequency of urinary infections, vaginal atrophy, kraurosis vulvae, hot flashes and night sweats, fatigue, emotional changes (mood swings and changes in sexual interest), sleep disturbances (insomnia), drier skin and hair, increased growth of facial and body hair, aches and pains in the joints, headaches, palpitations (rapid, irregular heart beats), vaginal itching, osteoporosis, and generalized itching.

The invention, therefore, is directed to a method of providing contraception to a female for the treatment of a condition or disorder, wherein the female is in need of treatment for the condition or disorder, by administering to the female the regimens disclosed herein. The female can be, for example, of childbearing age, peri-menopausal, or menopausal.

The invention is also directed to a method of providing contraception and treating a condition or disorder in a female, wherein the female is in need of both contraception and treatment of the condition or disorder, by administering to the female the regimens disclosed herein. The female can be, for example, of childbearing age or peri-menopausal.

The regimens disclosed herein can include administration to a female beginning at Day 1 of a menstrual cycle that is defined as beginning at the first day of menstrual flow.

Alternatively, the regimens disclosed herein can also include administration to a female beginning at Day 1 of a menstrual cycle that is defined as beginning at the day after the ending of the menstrual flow. Alternatively, the regimens disclosed herein also can include administration to a female beginning at Day 1 of a menstrual cycle that is defined as beginning at any day within the menstrual cycle.

As used herein, “female” refers to any animal classified as a mammal, including humans and non-humans, such as, but not limited to, domestic and farm animals, zoo animals, sports animals, and pets.

“Peri-menopausal female” refers to a woman who has not yet definitely arrived at menopause but who is experiencing symptoms associated with menopause. “Peri-menopause” means “about or around the time of menopause.” It encompasses the years preceding the last menstrual period during which ovarian function declines and ultimately ceases and can include the presence of symptoms and irregular cycles. “Menopausal female” refers to a woman who has definitely arrived at menopause and may be experiencing symptoms associated with menopause. Menopause or post-menopause is the permanent cessation of menstruation after the loss of ovarian activity and is generally defined clinically as the absence of menstruation for about one year. Menopause may occur naturally in a woman or it may be artificially induced, e.g., through surgical or chemical means. For example, removal of the ovaries, which can occur, e.g., through hysterectomy, frequently leads to symptoms associated with menopause (“surgical menopause”).

The terms “treat” and “treatment” refer to both therapeutic treatment and prophylactic or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state, remission (whether partial or total), whether detectable or undetectable; or enhancement or improvement of condition, disorder or disease. Treatment includes eliciting a clinically significant response, without excessive levels of side effects. Treatment also includes prolonging survival as compared to expected survival if not receiving treatment.

The term “continuous” or “consecutive” in reference to “administration” means that the frequency of administration is at least once daily. Note, however, that the frequency of administration can be greater than once daily and still be “continuous,” e.g., twice or even three times daily, as long as the dosage levels as specified herein are not exceeded.

The term “dosage level” means the total amount of estrogen or progestin administered per day. Thus, for example, “continuous administration” of a progestin to a woman at a “dosage level” of 30 μg means that the woman receives a total of 30 μg of progestin on a daily basis, whether the progestin is administered as a single 30 μg dose or, e.g., three separate 10 μg doses. A conventional means of continuously administering an estrogen or progestin is as a single daily oral dose at the prescribed dosage level.

For each of the disclosed methods of the invention, the effect of administration of the regimens with respect to the specified condition (e.g., inducing the specified condition in the female, reducing the occurrence of the condition, minimizing the condition, or treating the condition or disorder) can be evaluated in comparison to each other, to the condition or disorder exhibited by the female after administration of a standard 28-day contraceptive regimen other than the disclosed regimens, after administration of an extended cycle contraceptive regimen other than the disclosed regimens, and/or after administration of no contraceptive regimen. For example, the effect of administering the regimens to treat a menstrual bleeding disorder can be evaluated by comparing the occurrence and/or severity of the bleeding disorder in females suffering from the disorder who have been administered the regimens with the occurrence and/or severity of the bleeding disorder in females suffering from the disorder who have not been treated with a contraceptive regimen, or with females suffering from the disorder who have been administered a contraceptive regimen not disclosed in the present invention. The effect of administering the regimens of the invention can also be evaluated by comparing the occurrence and/or severity of a condition in a female before and after administration of the regimens of the invention, or by evaluating the condition of the female during the course of one or more cycles.

When the period of continuous administration of estrogen and progestin, which in some aspects of the invention is more than 50 consecutive days, such as about 60 to about 110 consecutive days, is followed by a hormone-free period or unopposed estrogen interval of about 2 to about 10 consecutive days, the extended cycle regimen is characterized by a reduced incidence of breakthrough or unscheduled bleeding by about the fourth cycle. When the extended cycle regimen is administered on a schedule of, e.g., 84 days of administration of the estrogen and progestin, followed by a hormone-free period or unopposed estrogen interval of, e.g., 7 days, the incidence of breakthrough bleeding decreases with continued use of the extended cycle regimen, so that by the fourth menstrual cycle (after about 274 days), it is comparable to that observed with the traditional 28-day regimen. Further continued use of the disclosed extended regimen can lead to even further reduction in the incidence of breakthrough bleeding. Thus, the present invention is directed to a method of reducing breakthrough bleeding in a female in need thereof by administering the extended cycle regimen disclosed herein to the female.

For example, the female can be of childbearing age or peri-menopausal.

The invention is also directed to a method of providing contraception and reducing breakthrough bleeding in a female in need thereof by administering to the female the extended cycle regimens disclosed herein. For example, the female can be of childbearing age or peri-menopausal.

The invention is also directed to methods of reducing unscheduled bleeding, such as breakthrough bleeding, and/or spotting, in a female by administering to the female a run-in regimen of the invention prior to administration of a lower dose hormone regimen.

The invention is directed to a method of inducing regular, predictable withdrawal bleeding in a female in need thereof by administering to the female the regimens disclosed herein. The female can be, but is not limited to, a female of childbearing age or a peri-menopausal female. Administration of the regimens is useful in controlling menstrual cycles in a female by inducing regular, predictable withdrawal bleeding. By suppressing ovulation and delivering estrogen and progesterone in a programmed fashion, the regimens can establish or restore synchrony to the endometrium. This is particularly useful in the treatment of heavy or intermenstrual bleeding. The resulting predictable timing and shorter duration of bleeding are especially advantageous to peri-menopausal women, who often experience irregular menstrual cycles.

The invention is also directed to a method of providing contraception and inducing regular, predictable withdrawal bleeding in a female in need thereof by administering to the female the regimen disclosed herein. The female can be, for example, a female of childbearing age or a peri-menopausal female.

The invention is directed to a method of reducing frequency or delaying onset of a menstrual cycle in a female in need of delayed or reduced menstruation by administering to the female the regimens disclosed herein. The female can be, but is not limited to, a female of childbearing age or a peri-menopausal female. Particular groups or subpopulations of women can benefit from reduced menstruation, such as women enlisted in the military and women athletes. Control of the menstrual cycle, or even induction of amenorrhea using the extended bridged cycle regimen, can be an advantage for women on active duty. The non-contraceptive benefits resulting from use of the regimens, such as reduction in dysmenorrhea, premenstrual syndrome, menorrhagia, iron deficiency anemia, and ability to control timing of withdrawal bleeding, can be desirable and advantageous to women athletes as well.

The invention is also directed to a method of providing contraception and reducing frequency or delaying onset of a menstrual cycle in a female in need thereof by administering to the female the regimens disclosed herein. The female can be, for example, a female of childbearing age or a peri-menopausal female.

The invention is directed to a method for minimizing uterine bleeding in a female in need thereof by administering to the female the regimens disclosed herein. The female can be, but is not limited to, a female of childbearing age or a peri-menopausal female.

By diminishing endometrial proliferation, administration of estrogen and progestin in the regimens can reduce the volume and duration of menstrual flow. A female on the disclosed extended regimen experiences fewer total scheduled days of bleeding than a female on a traditional 28-day regimen, and experiences less blood loss, because the regimens involves fewer stop/start transitions per year. The female to be treated can exhibit menorrhagia or abnormal uterine bleeding. Menorrhagia or abnormal uterine bleeding is often associated with conditions that include, but are not limited to, adenomyosis and uterine leiomyomas (uterine fibroids). As used herein, “abnormal uterine bleeding” refers to an abnormal duration of bleeding (i.e., greater than about 7 days of bleeding, or hypermenorrhea), abnormal amount of bleeding (i.e., greater than about 80 mL blood loss during menses, or menorrhagia), increased frequency of bleeding (i.e., less than about 22 days between menstrual cycles, or polymenorrhea), or any combinations thereof.

The invention is also directed to a method of providing contraception and minimizing uterine bleeding in a female in need thereof by administering to the female the regimens disclosed herein. The female can be, for example, a female of childbearing age or a peri-menopausal female.

The invention is, moreover, directed to a method of treating a menstrual bleeding disorder in a female in need thereof by administering to the female the regimens disclosed herein. The female can be, but is not limited to, a female of childbearing age or a peri-menopausal female.

The invention is also directed to a method of providing contraception and treating a menstrual bleeding disorder in a female in need thereof by administering to the female the regimens disclosed herein. The female can be, for example, a female of childbearing age or a peri-menopausal female.

The invention is directed to a method of treating symptoms associated with ovarian cysts, uterine leiomyomas (fibroids), or Polycystic Ovarian Syndrome in a female in need thereof by administering to the female the regimens disclosed herein. The female can be, for example, of childbearing age, peri-menopausal, or menopausal. The invention is also directed to a method of providing contraception and treating symptoms associated with ovarian cysts, uterine leiomyomas (fibroids), or Polycystic Ovarian Syndrome in a female in need thereof by administering to the female the regimens disclosed herein. The female can be, for example, a female of childbearing age or peri-menopausal.

Ovarian cysts, uterine leiomyomas (fibroids), or Polycystic Ovarian Syndrome can cause symptoms including, but not limited to, pelvic pain, dysmenorrhea, abnormal uterine bleeding, acne, and hirsutism. In the invention, such symptoms can be treated by administration of the regimens described herein.

Ovarian cysts arise from functional cysts that commonly occur around mid-cycle, when a follicle destined to become an egg fails to mature. Instead of leaving the ovary in a process known as ovulation, it remains inside, floating in a tiny sac of fluid. It is that sac that eventually forms into a cyst. Although rarely malignant, ovarian cysts lead to 200,000 hospitalizations in the United States each year. For some women, some studies have shown that the cysts develop cycle after cycle. Though ovarian cysts can sometimes be asymptomatic, they can also cause pain (constant pelvic pain, pain during intercourse, pain during pelvic movement, and/or pain before or after menses), abnormal bleeding (lengthened, shortened, irregular and/or absent menses), and/or abdominal bloating or distension.

Uterine fibroids are benign growths of uterine muscle that sometimes exist singly, but most often are multiple and range in size from microscopic to filling most of the lower abdominal cavity. Many women with fibroids have no symptoms at all. For those that do, the most common complaints are pressure symptoms and heavy, prolonged periods. There may be pressure in the pelvic region from the enlarged uterus, and the resulting symptoms are often related to where the fibroid is exerting pressure (e.g., increased urinary frequency, constipation or difficulty with bowel movements). The pressure can also cause backache, lower abdominal discomfort, and pain during and after intercourse. Fibroids can cause very heavy and prolonged periods, leading to iron-deficiency anemia, as well as painful periods (secondary dysmenorrhea). The presence of fibroids can also cause reproductive problems such as infertility, multiple miscarriages, premature labor, or labor complications.

The term “ovarian cyst” as used above represents more singular occurrences caused by the failure of an egg to mature. Polycystic Ovarian Syndrome (PCOS), in contrast, is due to an abnormal production of LH (luteinizing hormone) and FSH (follicle stimulating hormone) by the pituitary gland. An imbalance of these hormones stops egg production and increases production of androgens, with the ovaries producing higher levels of testosterone and estrogens. This results in ovaries “peppered” with empty egg follicles that become inflamed cysts, irregular or stopped periods (which in turn causes infertility), excess body hair growth, and acne on the face and body. PCOS often leads to obesity, diabetes and hypertension.

Polycystic Ovarian Syndrome is the cause of most cases of androgen-dependent hirsutism. See Rittmaster, R. S., Lancet 349:191-195 (1997). Hirsutism can be described as the growth of excessive hair in women on parts of the body where excessive hair is generally not present, e.g., on the back and chest. Most cases of hirsutism are androgen-dependent, i.e., result from a combination of increased androgen production by the body and increased skin sensitivity to androgens. Normally, small quantities of androgens are produced by the ovaries and the adrenal glands. However, in women suffering from Polycystic Ovarian Syndrome, androgen levels are elevated, which can lead to the development of androgen-dependent conditions such as, for example, pronounced forms of acne (e.g., acne papulopustulosa), androgenetic alopecia and mild forms of hirsutism. Oral contraceptives can suppress the ovarian production of androgens and are thus useful in the treatment of these androgen-dependent conditions.

Thus, the invention is also directed to a method of treating hirsutism in a female in need thereof, by administering to the female the regimens disclosed herein. The female can be, for example, of childbearing age, peri-menopausal, or menopausal.

The invention is also directed to a method of providing contraception and treating hirsutism in a female in need thereof, by administering to the female the regimens disclosed herein. The female can be, for example, a female of childbearing age or peri-menopausal.

The invention is further directed to a method of treating alopecia in a female in need thereof, by administering to the female the regimens disclosed herein. The female can be, for example, of childbearing age, peri-menopausal, or menopausal. The invention is also directed to a method of providing contraception and treating alopecia in a female in need thereof, by administering to the female the regimens disclosed herein. The female can be, for example, a female of childbearing age or peri-menopausal.

The invention is directed to a method of decreasing risk of iron deficiency anemia in a female in need thereof by administering to the female the regimens disclosed herein. The female can be, but is not limited to, a female of childbearing age or a peri-menopausal female. The reduction in the volume and duration of menstrual flow that results from administration of the regimens can lead to a reduction in the total loss of blood, thus improving the body's iron stores and reducing the morbidity associated with menorrhagia. This effect is particularly desirable in women with coagulation or bleeding disorders that include, but are not limited to, von Willebrand's disease, hemophilia, Factor XI deficiency, chronic anticoagulation, and thrombocytopenia.

The invention is also directed to a method of providing contraception and decreasing risk of iron deficiency anemia in a female in need thereof by administering to the female the regimens disclosed herein. The female can be, for example, a female of childbearing age or a peri-menopausal female.

The invention is directed to a method of treating a menstrual disorder in a female in need thereof by administering to the female the regimens disclosed herein. The female can be, but is not limited to, a female of childbearing age or a peri-menopausal female. Menstrual disorders include, but are not limited to, irregular cycles, dysmenorrhea (painful menstruation), mittelschmerz, and dysfunctional uterine bleeding, as well as premenstrual symptoms such as, but not limited to, those associated with premenstrual syndrome (PMS) or Premenstrual Dysphoric Disorder (PMDD).

The invention is also directed to a method of providing contraception and treating a menstrual disorder in a female in need thereof by administering to the female the regimens disclosed herein. The female can be, for example, a female of childbearing age or a peri-menopausal female.

During the luteal phase of the menstrual cycle, as many as 75% of women with regular menstrual cycles experience some symptoms of premenstrual syndrome (PMS), a recurring, cyclical disorder involving behavioral, emotional, social and physical symptoms (Steiner et al., Ann. Rev. Med. 48:447-455 (1997)). Behavioral, emotional and social symptoms include, but are not limited to, irritability, mood swings, depression, hostility and social withdrawal. Physical symptoms include, but are not limited to, bloating, breast tenderness, myalgia, migraines or headaches, abdominal pain, and fatigue. True PMS only occurs during the luteal phase of the menstrual cycle, with a symptom-free period during the follicular phase. The etiology of PMS is still unknown.

A subgroup of women with PMS, about 2% to about 9%, exhibit symptoms that are primarily related to a severe mood disorder. In these women, the diagnosis of Premenstrual Dysphoric Disorder (PMDD), as defined in the Fourth edition of the Diagnostic and Statistical Manual of Mental Disorders (DSM-IV), can be applied (see “Premenstrual Dysphoric Disorder,” in DSM-IV™: Diagnostic and Statistical Manual of Mental Disorders, 4^{th} Ed., American Psychiatric Association, Washington, D.C., pp. 715-718 (1994)). According to the DSM-IV, a woman with PMDD must have at least five premenstrual symptoms during the luteal phase, with at least one of the symptoms being an emotional or “core” symptom. The core symptoms must be irritability, anger, mood swings, tension or depression (and interfere with daily activities), and must be confirmed by a prospective daily rating for at least two cycles. Three to five percent of women with PMS report to have PMDD. There is also a subgroup of women who experience severe PMS, which accounts for about 20% of the PMS population. These women experience severe emotional symptoms that do not fall under the strict criteria of PMDD as defined in DSM-IV but require medical attention.

Suppression of ovulation that results from administration of the regimens can also eliminate mid-cycle pain (“mittelschmerz”) associated with rupture of the ovarian follicle. Additionally, suppression of ovulation and delivery of estrogen and progesterone in a regular, predictable schedule, which results from use of the regimens can be beneficial in the treatment of other menstrual disorders such as heavy or intermenstrual bleeding. In some aspects of the invention, the female is, but not limited to, a peri-menopausal female.

The invention is directed to a method of treating acne in a female in need thereof by administering to the female the regimens disclosed herein. The female can be, for example, of childbearing age, peri-menopausal, or menopausal. The regimens can suppress gonadotropin and decrease ovarian and adrenal androgen production, resulting in an improvement in acne.

The invention is also directed to a method of providing contraception and treating acne in a female in need thereof by administering to the female the regimens disclosed herein. The female can be, for example, a female of childbearing age or peri-menopausal.

The invention is directed to a method of treating endometriosis in a female in need thereof by administering to the female the regimens disclosed herein. The female can be, but is not limited to, a female of childbearing age or a peri-menopausal female. The invention is also directed to a method of providing contraception and treating endometriosis in a female in need thereof by administering to the female the regimens disclosed herein. The female can be, for example, a female of childbearing age or a peri-menopausal female.

In hormonal therapy of endometriosis, endometriotic tissue responds to adverse endocrine environments (low estrogen and/or high progestin concentration). Progestins produce marked atrophy of the endometrium and ectopic endometrial tissue and decrease intraperitoneal inflammation associated with endometriosis. The American College of Obstetrics and Gynecology stated that progestins, alone or in combination with estrogens as oral contraceptives, are an optimal choice for the management of endometriosis in women who desire contraception (American College of Obstetricians and Gynecologists, ACOG Practice Bulletin No. 11 (December 1999)). Since pain associated with endometriosis is often episodic and related to uterine bleeding, the use of the regimens of the present invention is beneficial for treating endometriosis.

Chronic pelvic pain often precedes and is associated with the development of endometriosis. Thus, the invention is also directed to methods of treating chronic pelvic pain in a female in need thereof by administering to the female the regimens disclosed herein. The female can be, for example, of childbearing age or peri-menopausal. The invention is also directed to a method of providing contraception and treating chronic pelvic pain in a female in need thereof by administering to the female the regimens disclosed herein. The female can be, for example, a female of childbearing age or a peri-menopausal female.

The invention is further directed to a method of reducing the risk of endometrial cancer in a female in need thereof by administering to the female the regimens disclosed herein. The female can be, for example, of childbearing age, peri-menopausal, or menopausal. The invention is also directed to a method of providing contraception and reducing the risk of endometrial cancer in a female in need thereof by administering to the female the regimens disclosed herein. The female can be, for example, a female of childbearing age or peri-menopausal.

The invention is directed to a method of reducing the risk of ovarian cancer in a female in need thereof by administering to the female the regimens disclosed herein. The frequency of ovulation and thereby the frequency of ovarian stimulation can be reduced, suppressed, or eliminated by use of the regimens. The female can be, for example, of childbearing age, peri-menopausal, or menopausal. The invention is also directed to a method of providing contraception and reducing the risk of ovarian cancer in a female in need thereof by administering to the female the regimens disclosed herein. The female can be, for example, a female of childbearing age, peri-menopausal, or menopausal.

The invention is further directed to a method of treating benign breast disease, including, but not limited to, fibrocystic breast disease, in a female in need thereof by administering to the female the regimens disclosed herein. The female can be, for example, of childbearing age or peri-menopausal. Roughly a third of all women between the ages of 30 and 50 will be diagnosed with fibrocystic breast disease or other benign breast conditions. Other terms for fibrocystic breast disease include benign breast disease and mammary dysplasia.

The invention is also directed to a method of providing contraception and treating benign breast disease in a female in need thereof by administering to the female the regimens disclosed herein. The female can be, for example, a female of childbearing age or peri-menopausal.

The invention is directed to a method of reducing the risk of colorectal cancer in a female in need thereof by administering to the female the regimens disclosed herein. The female can be, for example, of childbearing age, peri-menopausal, or menopausal. The regimens of the present invention can protect against colorectal cancer as a result of changes in bile synthesis and secretion due to the female hormones in the regimen, which can lead to a reduced concentration of bile acids in the colon. It has also been observed that estrogen inhibits the growth of colon cancer cells in vitro, and estrogen receptors have been identified in normal and neoplastic colon epithelial cells. See Fernandez, E., et al., British J Cancer 84:722-727 (2001). Thus, the regimens are beneficial in the prevention or reduction in the occurrence of colorectal cancer.

The invention is also directed to a method of providing contraception and reducing the risk of colorectal cancer in a female in need thereof by administering to the female the regimens disclosed herein. The female can be, for example, a female of childbearing age or peri-menopausal.

The invention is directed to a method of treating an infection in a female in need thereof by administering to the female the regimens disclosed herein. The female can be, for example, of childbearing age, peri-menopausal, or menopausal. For example, sexually transmitted diseases (STDs) are infections caused by a pathogen such as a virus, bacterium, parasite, or fungus, that is spread from person to person through sexual contact. STDs can be painful, irritating, and even life-threatening. The regimens can have a protective role against the development of some STDs because they stimulate the body to produce a thicker cervical mucous, which acts as a barrier to semen carrying bacteria that cause sexually transmitted diseases.

The invention is also directed to a method of providing contraception and treating an infection in a female in need thereof by administering to the female the regimens disclosed herein. The female can be, but is not limited to, a female of childbearing age or peri-menopausal.

Pelvic Inflammatory Disease (PID) is a complication that can result from STD infections. PID is a serious syndrome of the female reproductive tract that results from the spread of infections (most often sexually transmitted infections such as Chlamydia trachomatis and Nisseris gonnorrheoea) from the vagina and endocervix to the uterus, fallopian tubes and ovaries. PID is commonly manifested as endometritis (infection of the lining of the uterus) or salpingitis (infection of the fallopian tubes), and less commonly as pelvic peritonitis and/or inflammation of contiguous structures. (MacDonald, N. E., and Bowie, W. R., Canadian Communicable Disease Report 21S4: 25-33 (1995); Westrom, L. and Mardh, P-A., Sexually Transmitted Diseases, 2^{nd} Ed., pages 593-613, New York: McGraw-Hill, 1990).

PID is a major cause of infertility and ectopic pregnancy. Ectopic pregnancy results from the implantation of a fertilized ovum in the fallopian tube or in the abdominal cavity and is thought to be caused by ciliary dysfunction within the fallopian tube resulting from prior tubal infection with N. gonorrhoea and/or C. trachomatis, which often results in loss of ciliated epithelial cells from the fallopian tubes. It has been estimated that prior tubal infection with STD agents causes about 50% of the cases of ectopic pregnancy. (MacDonald, N. E., and Brunham, R., Canadian Journal of Human Sexuality 6(2):161-170 (1997).)

The regimens can have a protective role against the development of PID because they stimulate the body to produce thicker cervical mucous, which helps prevent semen carrying STD-causing bacteria from gaining access to the uterus and eventually causing PID and PID-related complications, such as ectopic pregnancy. Thus, the regimens of the present invention are useful in the prevention or reduction in occurrence of sexually transmitted diseases, Pelvic Inflammatory Disease, and ectopic pregnancy. Accordingly, the invention is directed to a method of treating a sexually transmitted disease or Pelvic Inflammatory Disease in a female in need thereof by administering to the female the regimens disclosed herein. The invention is also directed to a method of preventing ectopic pregnancy in a female in need thereof by administering to the female the regimens disclosed herein. The female can be, but is not limited to, a female of childbearing age or a peri-menopausal female.

The invention is directed to methods of providing contraception and treating a sexually transmitted disease or Pelvic Inflammatory Disease in a female in need thereof by administering to the female the regimens disclosed herein. The invention, moreover, is directed to methods of providing contraception and preventing ectopic pregnancy in a female in need thereof by administering to the female the regimens disclosed herein. The female can be, but is not limited to, a female of childbearing age or a peri-menopausal female.

In addition, use of the regimens, in comparison to the use of a conventional 28-day contraceptive regimen, can lead to a reduction in the reported occurrences of infection such as urinary tract infections, pharyngitis, upper respiratory tract infections, and sinusitus. Thus, the invention is further directed to a method of treating certain infections, such as urinary tract infections, pharyngitis, upper respiratory tract infections, and sinusitus, in a female in need thereof by administering to the female the regimens disclosed herein. The female can be, for example, of childbearing age, peri-menopausal, or menopausal. The invention is also directed to a method of providing contraception and treating certain infections, such as urinary tract infections, pharyngitis, upper respiratory tract infections, and sinusitus, in a female in need thereof by administering to the female the regimens disclosed herein. The female can be, for example, of childbearing age or peri-menopausal.

The invention is directed to a method of treating temporomandibular disorder in a female in need thereof by administering to the female the regimens disclosed herein. The female can be, for example, of childbearing age, peri-menopausal, or menopausal.

Temporomandibular disorders (TMD) are disorders of the jaw muscles, temporomandibular joints, and/or the nerves associated with chronic facial pain. The extended cycle regimen of the present invention is useful in the treatment of TMD. The invention is also directed to a method of providing contraception and treating temporomandibular disorder in a female in need thereof by administering to the female the regimens disclosed herein. The female can be, for example, of childbearing age or peri-menopausal.

The invention is directed to a method of treating a catamenial symptom in a female in need thereof by administering to the female the regimens disclosed herein. The female can be, for example, of childbearing age or peri-menopausal. Catamenial symptoms are those associated with conditions, disorders, or diseases that can worsen around the time of menses. Such conditions, disorders, or diseases include, but are not limited to, asthma, rheumatoid arthritis, migraine headaches, seizure disorders or epilepsy, multiple sclerosis, and diabetes. The invention is also directed to a method of providing contraception and treating a catamenial symptom in a female in need thereof by administering to the female the regimens disclosed herein. The female can be, for example, of childbearing age or peri-menopausal.

Arthritis is a prevalent chronic condition in women. Hormonal factors can influence the frequency and severity of arthritis. In some women, arthritis symptoms such as joint stiffness, swelling and pain peak during the postovulatory phase of the menstrual cycle, and cyclic changes in local antibody release, white blood cell subpopulations and altered pain perception have been proposed as possible mechanisms (Case, A. M. and Reid, R. L., Arch. Intern. Med. 158:1405-1412 (1998)). Estrogen administered as a single agent, and as part of a combined oral contraceptive has been reported to benefit some women (Kay, C. R. and Wingrave, S. J., Lancet 1:1437 (1983); Linos, A., et al., Lancet 1:1871 (1978)). Thus, use of the regimens is beneficial as a method of treating a catamenial symptom, such as, e.g., a symptom associated with rheumatoid arthritis, in a female in need thereof.

Approximately 60% of women with migraines report a relationship to menstruation (Case, A. M. and Reid, R. L., Arch. Intern. Med. 158:1405-1412 (1998)). Decreasing levels of estrogen during the late luteal phase of the menstrual cycle or abrupt withdrawal of estrogen as during the hormone-free period in women taking oral contraceptives are thought to trigger migraine attacks (Sulak P. J., et al., Obstet. Gynecol 95:261-266 (2000); Kudrow, L., Headache 15:36-49 (1975); Whitty, C. W. M., et al., Lancet 1:856-859 (1966)). Thus, use of the regimens is beneficial as a method of treating a catamenial symptom in a female in need thereof, such as, e.g., a migraine headache, in a female.

Catamenial epilepsy refers to seizure disorders that occur or worsen around menstruation. It is believed to result from cyclic alterations in both ovarian hormone levels and drug metabolism (Case, A. M. and Reid, R. L., Arch. Intern. Med. 158:1405-1412 (1998)). Thus, use of the regimens is beneficial as a method of treating a catamenial symptom such as, e.g., a symptom associated with epilepsy, in a female in need thereof.

The invention is directed to a method of treating headache or nausea unrelated to the menstrual cycle in a female in need thereof by administering to the female the regimens disclosed herein. The female can be, for example, of childbearing age, peri-menopausal, or menopausal. Use of the regimens, in comparison to the use of a conventional 28-day contraceptive regimen, can lead to a reduction in the reported occurrences of non-menstrual related headache and nausea. Thus, the disclosed regimens can be used as methods of preventing or treating non-menstrual related headache and nausea. The invention is also directed to a method of providing contraception and treating headache or nausea unrelated to the menstrual cycle in a female in need thereof by administering to the female the regimens disclosed herein. The female can be, for example, of childbearing age or peri-menopausal.

The invention is directed further to a method of treating depression unrelated to the menstrual cycle in a female in need thereof by administering to the female the regimens disclosed herein. The female can be, for example, of childbearing age, peri-menopausal, or menopausal. “Depression” is a term that is often used to refer to different forms of depressive disorders and includes major depression, bipolar disorder (sometimes called manic-depressive illness), and dysthymia, a less severe form of depression. Major depression is manifested by a combination of symptoms that interfere with the ability to work, study, sleep, eat and enjoy once pleasurable activities. Bipolar disorder, which is not nearly as prevalent as other forms of depressive disorders, is characterized by cycling mood changes. Dysthymia, a less severe type of depression, involves long-term, chronic symptoms that do not disable, but keep one from functioning well or from feeling well.

Depression also includes temporary sadness and loneliness often felt from time to time.

Use of the regimens, compared to use of a conventional 28-day contraceptive regimen, can lead to a reduction in the reported occurrences of non-menstrual related depression.

Thus, the disclosed regimens can be used as methods of preventing or treating non-menstrual related depression.

The invention is also directed to a method of providing contraception and treating depression unrelated to the menstrual cycle in a female in need thereof by administering to the female the regimens disclosed herein. The female can be, for example, of childbearing age or peri-menopausal.

The invention is further directed to a method of increasing contraceptive effectiveness in a female in need thereof by administering to the female the regimens disclosed herein. The female can be, but is not limited to, a female of childbearing age or a peri-menopausal female. A female in need of contraceptive effectiveness can be, but is not limited to, a higher weight female. A “higher weight female” refers to a human female weighing about 70 kg or more or having a body mass index (BMI) of greater than about 25. In a recent study of body weight and oral contraceptive failure, women weighing about 70.5 kg or more were reported to have a 60% higher risk of oral contraceptive failure (Holt, V. L., et al., Obstet. Gynecol. 99:820-827 (2002)). In a study utilizing the extended cycle regimen, women who weighed about 70 kg or more experienced the same contraceptive effectiveness as women on the same extended cycle regimen who weighed less than about 70 kg.

Thus, the invention is directed to a method of increasing contraceptive effectiveness in a higher-weight female in need thereof, by administering to the female the regimens disclosed herein. The invention is directed to a method of increasing the contraceptive effectiveness in a human female weighing about 70 kg or more, weighing 80 kg or more, or weighing 90 kg or more, by administering to the female the extended cycle regimen.

The disclosed regimens can also be used as a method of increasing the contraceptive effectiveness in a human female with a body mass index of greater than about 25, greater than about 30, or greater than about 35. Thus, the invention is also directed to a method of increasing the contraceptive effectiveness in a human female with a body mass index of greater than about 25, greater than about 30, or greater than about 35, by administering to the female the extended cycle regimen.

The invention is also directed to a method of increasing fertility in a female in need thereof, by administering to the female the regimens disclosed herein, followed by administration of an agent to induce ovulation in the female. The female can be, but is not limited to, a female of childbearing age or a peri-menopausal female.

It has been observed clinically that women who are taking oral contraceptives for anovulation often conceive when pills are missed, or shortly after discontinuing oral contraceptive treatment, most likely due to a “rebound effect” occurring in the ovary at least for a short period of time. Suppression of ovarian activity using oral contraceptive pills for 2-6 months may result in decreases in early follicular ovarian androgen production and LH and estradiol levels. Increased androgen levels have been shown to have adverse effects on folliculogenesis. These endocrine changes in the early follicular phase may be responsible for improved ovarian response to clomiphene or other treatments for anovulatory infertility. See Brannigan, E. F., and Estes, M. A., Am. J. Obstet. Gynecol. 188:1424-1430 (2003).

Examples of agents that induce ovulation, and that can be administered following discontinuation of the disclosed regimens, include, but are not limited to, menotropins (Follicle Stimulating Hormone (FSH) and Luteinizing Hormone (LH), e.g., Pergonal®) and chlomiphene citrate (Clomid®). The ovulation-inducing agent can be administered during a suitable time as can be determined by one of skill in the art, e.g., a physician. In some aspects of the invention, the ovulation-inducing agent can be administered, e.g., within about one week to about one month, or within about one week to about two weeks, after discontinuation of the regimens of the present invention. In some aspects of the invention, the ovulation-inducing agent is administered, e.g., about 2 to about 10 days, or about 5 to about 9 days after discontinuation of the regimens.

Thus, the invention is directed to a method of increasing fertility in a female in need thereof, the method comprising (i) administration to the female of the regimens disclosed herein; (ii) discontinuation of administration of the regimens; and (iii) optional administration to the female of an ovulation-inducing agent during the discontinuation of administration of the regimens; wherein fertility in the female is increased.

In some aspects of the invention, the disclosed regimens are particularly useful in peri-menopausal women and/or menopausal women. Peri-menopausal and menopausal women frequently experience a large variety of conditions and disorders that have been attributed to estrogen deprivation due to ovarian failure or hypoestrogenism. The duration of these disorders can be extremely variable and include hot flushes which can be devastating in some women and very mild in others. Dryness of the vagina associated with susceptibility to minor infections, and frequently associated with discomfort during intercourse, is another symptom that can be directly related to the decrease in estrogen availability.

In a long-term sense, one of the most health-threatening aspects of menopause is the loss of mineral from bone which can result in a decrease in bone mass (osteoporosis) and generates a serious risk of fractures. For example, evidence exists that there is a six-fold increase in fractures in post-menopausal women as opposed to men of the same age (Garraway et al. Mayo Clinic Proceedings 54:701-707 (1979)). These fractures, of course, carry a high complication rate among older people, a marked increase in disability and general morbidity, and certainly an increased risk of mortality.

Another serious health-threatening aspect of menopause is the impressive loss of protection against heart attacks, which is enjoyed by younger women up to the age of 60, when compared to men of the same age. The steep increase in mean serum cholesterol concentration, which occurs around menopause (during the fourth and fifth decades), can contribute importantly to the progressive increase in death from ischemic heart disease in older women. In the eighth and ninth decades, the incidence of deaths from ischemic heart disease, approaches that of men (Havlik, R. J. and Manning-Feinleid, P. H., NIH Publication No. 79-1610, U.S. Department of HEW (1979)).

Accordingly, the invention is directed to a method for treating conditions, such as the physical conditions described above, resulting from menopausal estrogen decline in a menopausal female in need thereof by administering the regimens disclosed herein to the female. The invention is also directed to a method for treating conditions, such as the physical conditions described above, resulting from hypoestrogenism in a female in need thereof by administering the regimens disclosed herein to the female. The invention is further directed to a method for treating conditions, such as the physical conditions described above, resulting from ovarian failure in a female in need thereof by administering the regimens disclosed herein to the female.

Additionally, menopausal hormone therapy is often associated with unscheduled bleeding. Unscheduled bleeding can result from endometrial bleeding due to changes in the ratio of endothelial growth factor (pro-angiogenic) to thrombospondin-1 (anti-angiogenic growth factor); alterations in matrix metalloproteinases and their tissue inhibitors; changes in endometrial haemostais due to tissue factor; and increased endometrial leucocytes (Hickey, M. Meopause Intl. 13(4): 188-190 (December 2007).

Unscheduled bleeding is common in both sequential and continuous combined hormone therapy users, with no established methods of reducing bleeding. Id.

Accordingly, the methods of the present invention are also directed to administration of a run-in regimen of the invention prior to lower dose hormone regimens, such as hormone therapy regimens administered to menopausal females, to reduce breakthrough bleeding in menopausal females in need thereof. Lower dose hormone therapy regimens include estrogen-only regimens, progestin-only regimens, and estrogen/progestin combination regimens. Doses associated with hormone therapy regimens may be lower than doses associated with contraceptive regimens. For example, the dose of ethinyl estradiol in hormone therapy regimens is generally about 5 times lower than the dose of ethinyl estradiol used in oral contraceptives. See, for example, Chandler, C., “Abbreviated Dug Class Review: Combined Estrogen and Progestin Products for Hormone Therapy” (Nov. 14, 2003), available at www.pbm.va.gov/reviews/CombinHT.pdf. Similarly, a dose of 0.625 mg conjugated estrogens as frequently administered in hormone therapy regimens corresponds to a dose of 5 μg of ethinyl estradiol. Id.

The invention is also directed to a method of providing contraception and treating conditions, such as the physical conditions described above, resulting from hypoestrogenism in a peri-menopausal female in need thereof by administering the regimens disclosed herein to the peri-menopausal female. The invention is further directed to a method of providing contraception and treating conditions, such as the physical conditions described above, resulting from ovarian failure in a peri-menopausal female in need thereof by administering the regimens disclosed herein to the peri-menopausal female.

In addition to the above-mentioned major physical problems, some menopausal and peri-menopausal women experience a large variety of other symptoms ranging from depression, insomnia, and nervousness, to symptoms of arthritis and so forth.

It is generally agreed that estrogen is the most effective agent for the control or prevention of menopausal flushes and vaginal atrophy. It is effective in retarding or preventing the appearance of clinical evidence of osteoporosis. In appropriate doses, when combined with progestin, a favorable effect upon blood lipids can also be seen. Problems with estrogen therapy do exist, however, and have been widely explored and documented in the medical literature. The means by which estrogen has been administered, generally speaking, involves either the use of estrogen alone or estrogen plus a progestin.

Estrogen alone, given in small doses on a continuous basis, is effective in most patients for the control of the above symptoms and problems associated therewith. However, although the vast majority of women taking continuous low-dose estrogen will not have bleeding for many months or even years, there is a distinct risk posed by this routine of silently (i.e., exhibiting no overt symptoms) developing “hyperplasia of the endometrium.” This term refers, of course, to an overstimulation of the lining of the uterus which can become pre-malignant, coupled with the possibility that the patient will eventually develop cancer of the uterine lining even under such a low-dose regimen (Gusberg et al. Obstet. Gynecol. 17:397-412 (1961)).

Estrogen alone can also be given in cycles, usually 21-25 days on treatment and 5-7 days off treatment. Again, if small doses of estrogen are required to control the symptoms and it is used to this fashion, only about 10% of women will experience withdrawal bleeding between the cycles of actual treatment. However, one must again be concerned by the risk of developing endometrial hyperplasia and by the increased relative risk of developing cancer of the uterus (Research on the Menopause: Report of a W.H.O. Scientific Group, 53-68 (1981)).

The addition of progestin with estrogen, however, as in the regimens disclosed herein, will virtually eliminate the concern about developing endometrial hyperplasia and reduce the risk of developing endometrial carcinoma below that of the untreated general population.

Thus, the invention is directed to a method of treating a menopausal disorder or a peri-menopausal symptom in a female in need thereof by administering to the female the regimens disclosed herein. The invention is also directed to a method of providing contraception and treating a peri-menopausal symptom in a peri-menopausal female in need thereof by administering to the female the regimens disclosed herein.

The regimens can be used as methods of maintaining bone density or preventing loss of bone density in a female. The regimens can also be used in this way by administering, e.g., calcium and/or vitamin D in combination with the administration of estrogen and progestin.

The regimens are not limited to administration to peri-menopausal or menopausal females as a method of maintaining bone density or preventing bone loss. The regimens can also be used in a method of maintaining bone density or preventing bone loss by administration to a female of childbearing age that is not peri-menopausal or menopausal. For example, the regimens can be used with females 12-16 years of age who have not yet achieved peak bone density, but who, due to various conditions such as anorexia, are at risk of loss of bone density or at risk of not achieving a normal physiologic bone density for age and developmental maturity.

Thus, the regimens can also be used as methods of treating a condition in a female in need thereof resulting from menopausal or peri-menopausal estrogen decline, including osteoporosis. The regimens can also be used as methods of providing contraception and treating a condition in a peri-menopausal female in need thereof resulting from peri-menopausal estrogen decline, including osteoporosis.

The regimens can also be used as methods of treating a female in need of hormone replacement therapy.

### Reduction of Unscheduled Bleeding

The invention is also directed to methods of reducing unscheduled bleeding, such as breakthrough bleeding, and/or spotting, associated with lower dose hormone regimens in a female by prior administration of one or more cycles of a run-in regimen as disclosed herein. The female can be, for example, of childbearing age, peri-menopausal, or menopausal.

In some aspects, the run-in regimen is administered for one cycle. In some aspects, the run-in regimen is administered for two cycles. In some aspects, the run-in regimen is administered for three, four, or five cycles.

In some aspects, the run-in regimen comprises one or more cycles of an extended cycle regimen as described above.

In some aspects, the run-in regimen comprises one or more cycles of an extended cycle regimen in which each cycle of the regimen comprises a combination of estrogen and progestin administered for 84 consecutive days, followed by a hormone-free period of 7 consecutive days.

In some aspects, the run-in regimen comprises one or more cycles of an extended cycle regimen in which each cycle of the regimen comprises a combination of estrogen and progestin administered for 84 consecutive days, followed by administration of an unopposed estrogen interval of 7 days.

In some aspects, the run-in regimen comprises one or more cycles of a 28-day regimen. In some aspects, at least one period in each cycle of the run-in regimen is about 21 to about 26, about 22 to about 25, about 23 to about 25, or about 25 to about 26 consecutive days. In some aspects, at least one period in each cycle of the run-in regimen is about 21 consecutive days. In some aspects, at least one period in each cycle of the run-in regimen is about 25 consecutive days. In some aspects, at least one period in each cycle of the run-in regimen is about 23 consecutive days. In some aspects, the run-in regimen further comprises a hormone-free period or an unopposed estrogen interval of about 2 to about 10 consecutive days. In some aspects, the hormone-free period or unopposed estrogen interval is about 3 to about 5 consecutive days, about 5 consecutive days, or about 3 consecutive days. In some aspects, the hormone-free or unopposed estrogen interval is about 5 to about 8, about 2 to about 7, about 3 to about 7, or about 7 consecutive days.

In some aspects, the run-in regimen comprises one or more cycles of a 28-day regimen in which each cycle of the regimen comprises a combination of estrogen and progestin administered for 25 consecutive days, followed by an unopposed estrogen interval of 3 consecutive days.

In some aspects, the run-in regimen comprises a regimen in which the combination of estrogen and progestin is administered in daily dosage amounts equivalent to about 30 μg of ethinyl estradiol, about 20 μg of ethinyl estradiol, about 150 μg of levonorgestrel, about 100 μg of levonorgestrel, and combinations thereof. In some aspects, the run-in regimen comprises a regimen in which the combination of estrogen and progestin is administered in daily dosage amounts equivalent to about 30 μg of ethinyl estradiol and about 150 μg of levonorgestrel. In some aspects, the run-in regimen comprises administration of an estrogen and progestin combination, followed by a administration of an unopposed estrogen interval comprising a daily dosage amount of estrogen equivalent to about 10 μg to about 30 μg of ethinyl estradiol. In some aspects, the run-in regimen comprises administration of an estrogen and progestin combination, followed by a administration of an unopposed estrogen interval comprising a daily dosage amount of estrogen equivalent to about 10 μg of ethinyl estradiol. In some aspects, the run-in regimen comprises administration of an estrogen and progestin combination, followed by a administration of an unopposed estrogen interval comprising a daily dosage amount of estrogen equivalent to about 20 μg of ethinyl estradiol. In some aspects, the run-in regimen comprises administration of an estrogen and progestin combination, followed by a administration of an unopposed estrogen interval comprising a daily dosage amount of estrogen equivalent to about 30 μg of ethinyl estradiol.

The invention is further directed to administration of a lower dose hormone regimen following the run-in regimen, in which the lower dose hormone regimen contains a lower daily dosage amount of estrogen, a lower daily dosage amount of progestin, or a lower daily dosage amount of estrogen and progestin as compared to the daily dosage amount administered during at least one period in each cycle of the run-in regimen. The lower dose hormone regimen can contain additional or fewer active agents than those present in the run-in regimen.

The lower dose hormone regimen can be used as a method of providing contraception to a female in need of contraception, a method of providing contraception to a female for the treatment of a condition or disorder as described above to a female in need of treatment of the condition or disorder, or a method of providing contraception and treating a condition or disorder in a female to a female in need of both contraception and treatment of the condition or disorder. The female can be, for example, of childbearing age, peri-menopausal, or menopausal.

In accordance with the present invention, a lower dose hormone regimen can be a combined estrogen/progestin regimen, an estrogen-only regimen in which estrogen is administered without progestin, or a progestin-only regimen in which progestin is administered without estrogen. The lower dose hormone regimen can contain one period per cycle of administration or more than one period per cycle of administration. For example, a lower dose hormone regimen can contain multiple periods within each cycle such as a period of combined estrogen/progestin administration preceded or followed by a period of estrogen-only or progestin-only administration. Each cycle can further contain one or more periods of combined estrogen/progestin administration in which different doses of estrogen and/or progestin in the combination are administered in some or all of the periods. Similarly, each cycle can further contain one or more periods of estrogen-only or progestin-only administration in which different doses of estrogen or progestin are administered in some or all of the periods.

In some aspects, the lower dose hormone regimen can be administered for more than 20 consecutive days, for greater than 20 consecutive days, or for at least 20 consecutive days. In some aspects, at least one period in each cycle of the lower dose hormone regimen is about 21 to about 26, about 22 to about 25, about 23 to about 25, or about 25 to about 26 consecutive days. In some aspects, at least one period in each cycle of the lower dose hormone regimen is about 21 consecutive days. In some aspects, at least one period in each cycle of the lower dose hormone regimen is about 25 consecutive days. In some aspects, at least one period in each cycle of the lower dose hormone regimen is about 23 consecutive days. In some aspects, the lower dose hormone regimen further comprises a hormone-free period or an unopposed estrogen interval of about 2 to about 10 consecutive days. In some aspects, the hormone-free period or unopposed estrogen interval is about 3 to about 5 or about 3 consecutive days. In some aspects, the hormone-free or unopposed estrogen interval is about 5 to about 8, about 2 to about 7, about 3 to about 7, or about 7 consecutive days.

In some aspects, the lower dose regimen is a hormone therapy regimen. Hormone therapy regimens can comprise one or more periods of combined estrogen/progestin administration, one or more periods of estrogen-only administration, one or more periods of progestin-only administration, or combinations thereof. Hormone therapy regimens include those classified as cyclic, cyclic-combined, continuous-cyclic, continuous long-cycle, continuous-combined, and intermittent-combined. See, for example, Menopause: The Journal of the North American Menopause Society 10(2): 113-132 (2003). An example of a monthly cyclic regimen is administration of estrogen from day 1 to day 25, followed by progestogen for at days 11 to 25 or days 16 to 25, followed by a hormone-free period of 3 to 6 days. An example of a monthly cyclic-combined regimen is administration of estrogen and progestogen for 25 days, followed by a hormone-free period of 5 days. An example of a monthly continuous-cyclic regimen is continuous daily usage of estrogen, with progestogen added cyclically for 10 to 14 days, such as beginning at day 1 or day 15. An example of a continuous long-cycle regimen is continuous estrogen administration with progestogen added cyclically every 3 to 6 months for 14 days. An example of a continuous combined regimen is daily administration of a fixed dosage of estrogen and progestogen. An example of an intermittent-combined regimen is administration of estrogen daily with progestogen administered intermittently in cycles of 3 days on and 3 days off. Other regimens involve daily administration of single hormones such as a fixed dose of estrogen, a fixed dose of progestogen, or changing doses of estrogen or progestogen.

Suitable hormone therapy products intended for administration after a run-in regimen of the invention include both combination and single ingredient products. Combination products include 1 mg micronized 17-beta-estradiol and 0.5 mg norethindrone acetate (including ACTIVELLA); 0.05 mg 17-beta-estradiol and 0.14 mg norethindrone acetate (including COMBIPATCH); 0.05 mg 17-beta-estradiol and 0.25 mg norethindrone acetate (including COMBIPATCH); 1 mg micronized 17-beta-estradiol and 0.09 mg norgestimate (including PREFEST); 0.005 mg ethinyl estradiol and 1 mg norethindrone acetate (including FEMHRT); 0.625 mg conjugated equine estrogens and 1.5 mg medroxyprogesterone acetate (including PREMPRO); 0.625 mg conjugated equine estrogens and 2.5 mg medroxyprogesterone acetate (including PREMPRO); 0.625 mg conjugated equine estrogens and 5 mg medroxyprogesterone acetate (including PREMPRO and PREMPHASE); 0.625 mg esterified estrogens and 1.25 mg methyltestosterone (including ESTRATEST); and 1.25 mg esterified estrogens and 2.5 mg methyltestosterone (including H.S. ESTRATEST). Suitable single ingredient hormone therapy products include micronized oral 17-beta-estradiol at 0.5 mg, 1.0 mg, and 2.0 mg dosages; 17-beta estradiol patches at 0.05 mg, 0.075 mg, 0.1 mg, 0.25 mg, and 0.375 mg dosages (including ALORA, CLIMARA, ESCLIM, ESTRADERM, VIVELLE, and VIVELLE-DOT); conjugated equine estrogens, oral at 0.3 mg, 0.45 mg, 0.625 mg, 0.9 mg, 1.25 mg, and 2.5 mg (including PREMARIN); medroxyprogesterone acetate, oral at 2.5 mg, 5 mg, and 10 mg (including PROVERA), synthetic conjugated estrogens, oral at 0.3 mg, 0.625 mg, 0.9 mg, 1.25 mg (including CENESTIN and ENJUVIA), esterified estrogens, oral at 0.3 mg, 0.625 mg, and 2.5 mg; estropipate, oral at 0.625 mg, 0.75 mg, 1.25 mg, 1.5 mg, 2.5 mg, and 3 mg (including OGEN).

In some aspects, the lower dose hormone regimen comprises a combination of estrogen and progestin that is administered for about 21 to about 26 consecutive days, in which the daily dosage amount of estrogen is equivalent to about 20 μg ethinyl estradiol and the daily dosage amount of progestin is equivalent to about 150 μg of levonorgestrel. In some aspects, the lower dose hormone regimen comprises a combination of estrogen and progestin that is administered for 21 consecutive days, in which the daily dosage amount of estrogen is equivalent to about 20 μg ethinyl estradiol and the daily dosage amount of progestin is equivalent to about 150 μg of levonorgestrel.

In some aspects, the lower dose hormone regimen can be an extended regimen, including an extended regimen as disclosed herein. The lower dose hormone regimen can be administered for more than 50 consecutive days, for greater than 50 consecutive days, or for at least 50 consecutive days. In some aspects, the lower dose hormone regimen can be administered for about 60 to about 110 consecutive days, about 81 to about 89 consecutive days, at least 81 consecutive days, or at least 84 consecutive days.

In some aspects, the period of administration of the lower dose hormone regimen can be about one year, more than one year but less than two years, two years, or more than two years.

In some aspects, the lower dose hormone regimen comprises an extended cycle regimen in which the daily dosage amount of estrogen is less than the daily dosage amount of estrogen administered during at least one period in each cycle of the run-in regimen. In some aspects, the lower dose hormone regimen comprises a combination of estrogen and progestin that is administered for 84 consecutive days, in which the daily dosage amount of estrogen is equivalent to about 20 μg of ethinyl estradiol.

In some aspects, the lower dose hormone regimen comprises an extended cycle regimen in which the daily dosage amount of progestin is less than the daily dosage amount of progestin administered during at least one period in each cycle of the run-in regimen. In some aspects, the lower dose hormone regimen comprises a combination of estrogen and progestin that is administered for 84 consecutive days, in which the daily dosage amount of progestin is equivalent to about 100 μg of levonorgestrel.

In some aspects, the lower dose hormone regimen comprises an extended cycle regimen in which the daily dosage amounts of estrogen and progestin are less than the daily dosage amount of estrogen and progestin administered during at least one period in each cycle of the run-in regimen. In some aspects, the lower dose hormone regimen comprises a combination of estrogen and progestin that is administered for 84 consecutive days, in which the daily dosage amount of estrogen is equivalent to about 20 μg ethinyl estradiol and the daily dosage amount of progestin is equivalent to about 100 μg of levonorgestrel.

In some aspects, the lower dose hormone regimen further comprises a hormone free period of 7 consecutive days following administration of the combination of estrogen and progestin.

In some aspects, the lower dose hormone regimen further comprises an unopposed estrogen interval administered for 7 consecutive days following administration of the combination of estrogen and progestin. In some embodiments, the lower dose hormone regimen further comprises an unopposed estrogen interval administered for 7 consecutive days following administration of the combination of estrogen and progestin, in which the daily dosage amount of estrogen in the unopposed estrogen interval is equivalent to about 10 μg of ethinyl estradiol.

In some aspects, the run-in regimen is a conventional or commercial regimen in which the daily dosage amount of estrogen, the daily dosage amount of progestin, or the daily dosage amount of estrogen and progestin is higher than the daily dosage amount during at least one period in each cycle of the lower dose hormone regimen. In some aspects, the lower dose hormone regimen comprises a conventional or commercial hormone regimen, in which the daily dosage amount of estrogen, the daily dosage amount of progestin, or the daily dosage amount of estrogen and progestin is lower than the daily dosage amount administered during at least one period in each cycle of the run-in regimen.

In some aspects, the run-in regimen and/or lower dose regimen is selected from a commercially available regimen. Commercial combined estrogen/progestin formulations that are administered for 21 days followed by a 7 day hormone-free period with or without administration of placebo include: 20 μg ethinyl estradiol/1000 μg norethindrone acetate (LOESTRIN, LOESTRIN 1/20, MICROGESTIN, JUNEL); 20 μg ethinyl estradiol/100 μg levonorgestrel (ALESSE, AVIANE, LESSINA, LUTERA, SRONYX); 20 μg ethinyl estradiol/150 μg desogestrel (MERCILON, KARIVA); 20 μg ethinyl estradiol/75 μg gestodene (FERMODETTE); 20 μg ethinyl estradiol/3000 μg drosperinone (YAZ); 30 μg ethinyl estradiol/1500 μg norethindrone acetate (LOESTRIN 30, LOESTRIN 1.5/30, MICROGESTIN 1.5/30, JUNEL 1.5/30; 30 μg ethinyl estradiol/300 μg norgestrel (LO/OVRAL, LOW-ORGESTREL, CRYSELLE); 30 μg ethinyl estradiol/150 μg levonorgestrel (ORVANETTE, MICROGYNON 30, MIROGYNON 30 ED, NORDETTE, LEVLEN, LEVORA, PORTIA); 30 μg ethinyl estradiol/150 μg desogestrel (MARVELON, DESOGEN, ORTHO-CEPT, APRI); 30 μg ethinyl estradiol/75 μg gestodene (FEMODENE, FEMODENE ED, MINULET); 30 μg ethinyl estradiol/3000 μg drosperinone (YASMIN); 35 μg ethinyl estradiol/400 μg norethindrone (FEMCON Fe, OVCON 35, BALZIVA); 35 μg ethinyl estradiol/500 μg norethindrone (OVYSMEN, BREVINOR, MODICON, BREVICON, NORTREL 0.5/35); 35 μg ethinyl estradiol/1000 μg norethindrone (NOIMIN, ORTHO-NOVUM 1/35, NORINYL 1/35, NECON, NORTREL 1/35); 35 μg ethinyl estradiol/1000 μg ethynodiol diacetate (DEMULEN 1/35, ZOVIA 1/35, KELNOR); 35 μg ethinyl estradiol/250 μg norgestimate (CILEST, ORTHO CYCLEN, MONONESSA, SPRINTEC); 35 μg ethinyl estradiol/2000 μg cyproterone acetate (for severe acne or severe hirsutism: DIANETTE); 50 μg mestranol (equivalent to 35 μg ethinyl estradiol)/1000 μg norethindrone (NORINYL-1, ORTHO-NOVUM 1/50, NORINYL 1/50, NECON 1/50); 50 μg ethinyl estradiol/1000 μg norethindrone (OVCON 50); 50 μg ethinyl estradiol/1000 μg ethynodiol diacetate (DEMULEN 1/50, ZOVIA 1/50); 50 μg ethinyl estradiol/500 μg norgestrel (OGESTREL); and 50 μg ethinyl estradiol/250 μg levonorgestrel (NORDIOL). Other combined estrogen/progestin commercial products include: 20 μg ethinyl estradiol/90 μg levonorgestrel (LYBREL [365 days continuous administration, no placebo]); 20 μg ethinyl estradiol/1000 μg norethindrone acetate (LOESTRIN 24 Fe [24 days combined formulation, 4 days ferrous fumarate only]); 20 μg ethinyl estradiol/150 μg desogestrel (MIRCETTE [21 days combined formulation, 2 days placebo, 5 days 10 μg ethinyl estradiol only]); ethinyl estradiol/norgestimate combination with 7 tablets 25 μg/180 μg, 7 tablets 25 μg/215 μg, 7 tablets 25 μg/250 μg followed by 7 placebos (ORTHO TRI-CYCLEN LO); ethinyl estradiol/desogestrel combination with 7 tablets 25 μg/100 μg, 7 tablets 25 μg/125 μg, 7 tablets 25 μg/150 μg, followed by 7 tablets of ferric oxide (CYCLESSA, VELIVET); ethinyl estradiol/norethindrone acetate combination with 5 tablets 20 μg/1000 μg, 7 tablets 30 μg/1000 μg, 9 tablets 35 μg/1000 μg, followed by 7 tablets of ferrous fumarate 75 mg (ESTROSTEP Fe); ethinyl estradiol/levonorgestrel combination with 6 tablets 30 μg/50 μg, 5 tablets 40 μg/75 μg, 10 tablets 30 μg/125 μg, followed by a 7 day hormone-free period with or without placebo (TRINORDIOL, LOGYNON, LOGYNON ED, TRI-LEVLEN, TRIPHASIL, TRIVORA, ENPRESSE); ethinyl estradiol/gestodene combination with 6 tablets 30 μg/50 μg, 5 tablets 40 μg/70 μg, 10 tablets 30 μg/100 μg (TRIADENE, TRI-MINULET); ethinyl estradiol/norethindrone combination with 7 tablets 35 μg/500 μg, 9 tablets 35 μg/1000 μg, 5 tablets 35 μg/500 μg (SYNPHASE, TRI-NORINYL, LEENA); ethinyl estradiol/norethindrone combination with 7 tablets 35 μg/500 μg, 7 tablets 35 μg/750 μg, 7 tablets 35 μg/1000 μg (TRINOVUM, ORTHO-NOVUM 7/7/7, NECON 7/7/7, NORTREL 7/7/7); ethinyl estradiol/norgestimate combination with 7 tablets 35 μg/180 μg, 7 tablets 35 μg/215 μg, 7 tablets 35 μg/250 μg followed by 7 placebos (ORTHO TRI-CYCLEN, TRINESSA, TRI-SPRINTEC); ethinyl estradiol/norethindrone combination with 10 tablets 35 μg/500 μg, 11 tablets 35 μg/1000 μg, followed by 7 placebos (ORTHO-NOVUM 10/11, NECON 10/11); ethinyl estradiol/norethindrone combination with 7 tablets 35 μg/500 μg, 14 tablets 35 μg/1000 μg (BINOVUM). Commercial progestin-only pills include 350 μg norethindrone (MICRONOR, NORIDAY, NOR-QD, NORA-BE, JOLIVETTE, CAMILA, ERRIN); 500 μg ethynodiol diacetate (FEMULEN); 30 μg levonorgestrel (NORGESTON); and 75 μg desogestrel (CERAZETTE).

In some aspects, the run-in regimen is administered to a female who is not currently being administered a lower dose hormone regimen and prior to administration of a lower dose hormone regimen, wherein administration of the run-in regimen reduces unscheduled bleeding and/or spotting that would otherwise result from administration of the lower dose hormone regimen in absence of pretreatment with the run-in regimen.

In some aspects, the run-in regimen is administered to a female who is currently being administered a lower dose hormone regimen, such as a contraceptive regimen or hormone therapy regimen, but prior to administration of the next cycle of the lower dose hormone regimen, wherein administration of the run-in regimen reduces the occurrence of unscheduled bleeding and/or spotting resulting from administration of the lower dose hormone regimen.

In some aspects, the duration of administration of the run-in regimen is based on the time required for an individual female to attain cycle control. In some aspects, administration of the lower dose regimen begins after cycle control has been attained during administration of the run-in regimen. In some aspects, the duration of administration of the run-in regimen is based on an individual's prior history of cycle control using oral contraceptives. In some aspects, the duration of administration of the run-in regimen is reduced when the run-in regimen is administered to a female who has demonstrated early cycle control when using an oral contraceptive as compared to a female who has demonstrated more delayed cycle control when using an oral contraceptive.

In the extended regimens, run-in regimens, and/or lower dose hormone regimens comprising a combination of estrogen and progestin, the combined dosage form of estrogen and progestin can be administered monophasically, biphasically, triphasically, or multiphasically. As used herein, “monophasic” refers to the continuous use of one particular dose of estrogen and progestin during the period of administration of the combined dosage form of estrogen and progestin. “Biphasic” refers to administration of a first continuous dose of estrogen and progestin during a first portion of the period of administration of the combined dosage form of estrogen and progestin, with administration of a second continuous dose of estrogen and progestin during the second portion of the period of administration of the combined dosage form. “Triphasic” refers to administration of first, second, and third continuous doses of estrogen and progestin during the first, second, and third portions, respectively, of the period of administration of the combined dosage form of estrogen and progestin. “Multiphasic” refers to administration of four or more continuous doses of estrogen and progestin during the first, second, third, and fourth or more portions, respectively, of the period of administration of the combined dosage form of estrogen and progestin.

### Dosages and Formulations

The regimens described herein can include a daily dosage amount of estrogen equivalent to about 5 μg to about 50 μg of ethinyl estradiol. In some aspects of the invention, the regimens can include a daily dosage amount of estrogen equivalent to about 5 μg to about 25 μg of ethinyl estradiol. In other aspects of the invention, the regimens can include a daily dose of estrogen equivalent to about 25 μg to about 40 μg of ethinyl estradiol. In yet other aspects of the invention, the regimens can include a daily dose of estrogen equivalent to about 10 μg to about 30 μg of ethinyl estradiol. In some aspects of the invention, the regimens can include a daily dose of estrogen equivalent to about 20 μg of ethinyl estradiol.

The regimens of the invention can include a daily dosage amount of progestin equivalent to about 0.01 mg to about 1.5 mg of levonorgestrel. In some aspects of the invention, the regimens can include a daily dosage amount of progestin equivalent to about 0.01 mg to about 0.25 mg of levonorgestrel. In other aspects of the invention, the regimens can include a daily dose of progestin equivalent to about 0.05 mg to about 0.20 mg of levonorgestrel. In other aspects, the regimens can include a daily dose of progestin equivalent to about 0.15 mg of levonorgestrel. In yet other aspects of the invention, the regimens can include a daily dose of progestin equivalent to about 0.1 mg of levonorgestrel.

In some aspects of the invention, the estrogen and progestin of the regimens can be ethinyl estradiol and levonorgestrel, respectively, although other useful estrogens and progestins can be employed. The weight ratio of estrogen and progestin can be about 1:0.2 to about 1:300. In some aspects of the invention, the weight ratio of estrogen and progestin is about 1:1 to about 1:50. In other aspects of the invention, the weight ratio of estrogen and progestin is about 1:1 to about 1:10. For example, the daily amount of ethinyl estradiol is about 10 μg to about 30 μg and the daily amount of levonorgestrel is about 0.05 mg to about 0.2 mg.

The above dosages are not limiting. As will readily be understood, a lower dose hormone regimen as described herein will have a lower daily dosage amount than the daily dosage amount in at least one period in each cycle of the preceding run-in regimen.

The values given above are for ethinyl estradiol and levonorgestrel, and if a different estrogen or progestin is employed, an adjustment in the amount based on the relative potency or activity can be made. Correlations in potency among the various estrogens and among the various progestins are known. See, for example, EP 0 253 607, which is hereby incorporated in its entirety by reference. For example, in a contraceptive regimen, 30 μg of ethinyl estradiol is roughly equivalent to about 60 μg of mestranol or about 2,000 μg of 17β-estradiol. Similarly, 0.050 mg of levonorgestrel is roughly equivalent to about 0.175 mg of norethindrone acetate, about 0.050 mg of desogestrel, about 0.050 mg 3-ketodesogestrel, about 0.035 mg of gestodene, or about 0.100 mg of norgestrel. It should be understood that when norgestrel is used in place of levonorgestrel, its concentration is twice that of levonorgestrel. Norgestrel (dl-norgestrel) is a racemic mixture of optically active isomers, while levonorgestrel is one of the optically active isomers present in norgestrel.

Equivalent concentrations of estrogens and of progestins can be determined using either in vitro or in vivo assay methods. See, for example, Kuhl, H., Drugs 51(2):188-215 (1996); Philibert, D., et al., Gynecol. Endocrinol. 13:316-326 (1999); and Lundeen, S., et al., J Steroid Biochem. Molec. Biol. 78:137-143 (2001), in which the relative potencies of various progestins are compared using both in vitro and in vivo test assays. See also, for example, Dickey, R. P., “Contraceptive Therapy,” OBG Management Supplement (October 2000), pp. 2-6. Each of these documents is hereby incorporated by reference in its entirety.

For example, various combinations of progestin and estrogen that have been used in oral contraceptives are shown in Table 1.

TABLE 1
Combinations of Progestin and Estrogen

| | Norethindrone | | | | | |
|---|---|---|---|---|---|---|
| | Equivalent | | | EE Equivalent | | |
| | Dose | | | Dose | Dose | |
| Progestin | (mg) | Dose (mg) | Estrogen | (mg) | (mg) | P/E Ratio |
| Norethynodrel | 9.85 | 9.85 | Mestranol | 0.150 | 0.105 | 93.810 |
| | 5.00 | 5.00 | | 0.075 | 0.053 | 95.238 |
| | 2.50 | 2.50 | | 0.036 | 0.025 | 99.206 |
| | 2.50 | 2.50 | | 0.100 | 0.070 | 35.714 |
| Norethindrone | 10.00 | 10.00 | Mestranol | 0.060 | 0.042 | 238.095 |
| | 2.00 | 2.00 | | 0.100 | 0.070 | 28.571 |
| | 1.00 | 1.00 | | 0.050 | 0.035 | 28.571 |
| | 1.00 | 1.00 | | 0.080 | 0.056 | 17.857 |
| Norethindrone | 1.00 | 1.00 | Ethinyl | 0.050 | 0.050 | 20.000 |
| | 1.00 | 1.00 | estradiol | 0.035 | 0.035 | 28.571 |
| | | | (EE) | | | |
| | 0.50 | 0.50 | | 0.035 | 0.035 | 14.286 |
| | 0.40 | 0.40 | | 0.035 | 0.035 | 11.429 |
| Norethindrone | 2.50 | 2.50 | EE | 0.050 | 0.050 | 50.000 |
| acetate | 1.00 | 1.00 | | 0.050 | 0.050 | 20.000 |
| | 0.60 | 0.60 | | 0.030 | 0.030 | 20.000 |
| | 1.50 | 1.50 | | 0.030 | 0.030 | 50.000 |
| | 1.00 | 1.00 | | 0.020 | 0.020 | 50.000 |
| Ethynodiol | 1.00 | 1.00 | Mestranol | 0.100 | 0.070 | 14.286 |
| diacetate | | | | | | |
| Ethynodiol | 1.00 | 1.00 | EE | 0.050 | 0.050 | 20.000 |
| diacetate | 1.00 | 1.00 | | 0.035 | 0.035 | 28.571 |
| dl-Norgestrel | 0.50 | 0.75 | EE | 0.050 | 0.050 | 10.000 |
| | 0.30 | 0.45 | | 0.030 | 0.030 | 10.000 |
| Levonorgestrel | 0.10 | 0.35 | EE | 0.020 | 0.020 | 5.000 |
| | 0.15 | 0.52 | | 0.030 | 0.030 | 5.000 |

Equivalencies
50 mg Mestranol = approx. 35 mg Ethinyl estradiol (EE)
0.1 mg dl-Norgestrel = approx. 0.15 mg Norethindrone

Each block in Table 1 describes a specific combination of progestin and estrogen, e.g., norethynodrel and mestranol, and within each block older combinations are listed first, with successively newer combinations following.

Suitable progestins for use in the present invention include, but are not limited to, natural and synthetic compounds having progestational activity, such as, for example, progesterone, chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens. Prodrugs of suitable progestins can also be used in the extended cycle regimen of the present invention.

The expression “prodrug” denotes a derivative of a known direct acting drug, which derivative has enhanced delivery characteristics and therapeutic value as compared to the drug and is transformed into the active drug by an enzymatic or chemical process. Ethynodiol diacetate, which is converted in vivo to norethindrone, is an example of a progestin prodrug that can be used in the present invention. Additional examples of progestin prodrugs include, but are not limited to, norgestimate (which is converted in vivo to 17-deacetyl norgestimate, also known as norelgestromin), desogestrel (which is converted in vivo to 3-keto desogestrel, also known as etonogestrel), and norethindrone acetate (which is converted in vivo to norethindrone).

Suitable estrogens in the present invention include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); mestranol; agonistic anti-estrogens; and selective estrogen receptor modulators. Prodrugs of suitable estrogens can also be used in the extended cycle regimen of the present invention. Examples of estrogen prodrugs that can be used in the present invention include, but are not limited to, estradiol acetate (which is converted in vivo to 17β-estradiol) and mestranol (which is converted in vivo to ethinyl estradiol).

The antidepressant that is optionally combined with the regimens disclosed herein can be a selective serotonin reuptake inhibitor (SSRI), a tricyclic antidepressant or anxiolytic, or any antidepressant known to one of skill in the art. Suitable antidepressants include, but are not limited to, alprazolam (XANAX®), clomipramine (ANAFRANIL®), fluoxetine (PROZAC®), paroxetine (PAXIL®), sertraline (ZOLOFT®), nefazodone (SERZONE®), fenfluramine (PONDIMIN®) and venlafaxine (EFFEXOR®).

The daily amount of antidepressant administered can vary, depending on the antidepressant used, from about 0.75 to about 2 mg, from about 10 to about 20 mg, or from about 50 to about 100 mg. For example, in some aspects of the invention, fluoxetine hydrochloride is administered in a daily amount of about 5 mg to about 120 mg.

In some aspects of the invention, the antidepressant is administered during a hormone-free or unopposed estrogen interval of the regimens. In other aspects of the invention, the antidepressant is administered continuously throughout the regimens, intermittently, one time during each menstrual cycle, or once weekly. For example, in some aspects of the invention, fluoxetine hydrochloride is administered in a one-time or once-weekly dose of about 90 mg.

The estrogen and progestin are administered in the conventional manner by any route where they are active. For example, administration can be by, but is not limited to, parenteral, subcutaneous, intravenous, intramuscular, intraperitoneal, transdermal, buccal, or ocular routes, or intravaginally, by inhalation, by depot injections, or by hormone implants. Thus, modes of administration for the estrogen and progestin (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Most estrogens and progestins are orally active and this route of administration can be used in the invention. Accordingly, administration forms can include, but are not limited to, tablets, dragees, capsules and pills, which contain the estrogen and the progestin and one or more suitable pharmaceutically acceptable carriers.

For oral administration, the estrogen and progestin can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the estrogen and progestin compositions can take the form of, e.g., tablets or lozenges formulated in a conventional manner.

For administration by inhalation, the estrogen and progestin for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The estrogen and progestin can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The pharmaceutical compositions of the estrogen and progestin also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

In transdermal administration, the estrogen and progestin components, for example, can be applied to a plaster, or can be applied by transdermal, therapeutic systems that are consequently supplied to the organism.

The combination of estrogen and progestin can also be administered in combination with other active ingredients. For example, estrogen and progestin can be administered with vitamin D and/or calcium in the extended cycle regimen as a method of maintaining or preventing loss of bone density. The form of vitamin D and of calcium used in the present invention would be well known to those of skill in the art, as would the amount. For example, calcium can be administered in the form of calcium carbonate, at a daily dosage level of 500 mg.

Thus, pharmaceutical formulations containing the estrogen and progestin and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of estrogen and progestin as taught in this invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, “Modern Pharmaceutics”, Banker & Rhodes, Marcel Dekker, Inc. 1979; and “Goodman & Gilman's The Pharmaceutical Basis of Therapeutics,” 6^{th} Edition, MacMillan Publishing Co., New York 1980 can be consulted.

The run-in and lower dose hormone preparations can be produced in the form of one or more kits or packages, with the daily dosages arranged for proper sequential administration. For example, in some aspects of the invention, e.g., in the oral form of the formulation, the present invention provides a pharmaceutical package which contains combination-type contraceptives in multiple dosage units in a synchronized, fixed sequence, wherein the sequence or arrangement of the dosage units corresponds to the stages of daily administration. In other aspects, the run-in preparation can be produced in the form of a kit or package as disclosed herein for administration prior to a commercially available lower dose hormone regimen.

The pharmaceutical formulations useful in the invention can be provided in kit form containing at least about 50 tablets intended for ingestion on successive days, followed by about 2 to about 10 tablets, intended for ingestion on successive days.

Administration is daily for at least 50 consecutive days using tablets containing both the estrogen and the progestin, and is followed by administration that is daily for about 2 to about 10 consecutive days using hormone-free placebo tablets or tablets containing estrogen without progestin. For example, administration can be for 60-110 consecutive days, using tablets containing both estrogen and the progestin, followed by administration for at least 2-10 days, using hormone-free placebo tablets or tablets containing estrogen without progestin.

The pharmaceutical formulations can be provided in kit form containing, e.g., for a 91-day regimen, 84 tablets, each tablet containing estrogen and progestin, intended for ingestion on successive days, followed by 7 hormone-free placebo tablets or tablets containing estrogen without progestin, intended for ingestion on successive days.

The pharmaceutical formulations useful in the invention can be provided in kit form containing at least about 20 tablets intended for ingestion on successive days, followed by about 2 to about 10 tablets, intended for ingestion on successive days.

Administration is daily for at least 20 consecutive days using tablets containing the both the estrogen and the progestin, and is followed by administration that is daily for about 2 to about 10 consecutive days using hormone-free placebo tablets or tablets containing estrogen. For example, administration can be for 21-26 consecutive days, using tablets containing both estrogen and the progestin, followed by administration for at least 2-10 days, using hormone-free placebo tablets or tablets containing estrogen. As yet another example, administration can be for 21 days, using tablets containing both estrogen and progestin, followed by administration for 7 days, using hormone-free placebo tablets or tablets containing estrogen. In another example, administration can be for 25 consecutive days, using tablets containing both estrogen and the progestin, followed by administration for 3 days, using hormone-free placebo tablets or tablets containing estrogen. In another example, administration can be for 23 consecutive days, using tablets containing both estrogen and the progestin, followed by administration for 5 days, using hormone-free placebo tablets or tablets containing estrogen.

The run-in regimen and lower dose hormone regimens can be provided in the same kit or separate kits, with instructions for administering the run-in regimen prior to the lower dose hormone regimen.

In other aspects of the invention, the pharmaceutical formulations can be provided in kit form with tablets containing both the estrogen and the progestin, intended for ingestion on successive days, and 2 to 10 tablets, each tablet containing both placebo and an antidepressant or both estrogen and an antidepressant, e.g., fluoxetine hydrochloride, intended for ingestion on successive days.

All of the various aspects, embodiments and options described herein can be combined in any and all variations. The regimens disclosed herein can be administered to females of child-bearing age, peri-menopausal females, or menopausal females as needed for treatment of any of the conditions and disorders described above.

The following examples are illustrative, but not limiting, of the method and compositions of the present invention. Other suitable modifications and adaptations of the variety of conditions and parameters normally encountered and obvious to those skilled in the art are within the spirit and scope of the invention. Thus, the breadth and scope of the present invention should not be limited by any of the above-described exemplary embodiments, but should be defined only in accordance with the following claims and their equivalents.

### Example 1

This example provides detailed results of a randomized clinical study evaluating two 91-day extended cycle oral contraceptives (OCs): (1) a 91-day extended cycle OC containing 84 days of 30 μg ethinyl estradiol (EE) and 150 μg levonorgestrel (LNG) followed by 7 days of placebo, referred to as the “30 μg EE/150 μg LNG 91-day extended cycle OC” or the “30 μg EE/150 μg LNG extended cycle regimen,” and (2) a 91-day extended cycle OC containing 84 days of 20 μg EE and 100 μg LNG followed by 7 days of placebo, referred to as the “20 μg EE/100 μg LNG 91-day extended cycle OC” or the “20 μg EE/100 μg LNG extended cycle regimen.”

### Materials and Methods

Study Design and Population

This was a parallel, randomized, multicenter, open-label study of a 91-day extended cycle OC (30 μg EE/150 μg LNG) and Nordette® (30 μg EE/150 μg LNG).

Eligible women were randomized in a 2:1 fashion to the 91-day extended cycle OC or Nordette®. Study therapy was administered for one year (four consecutive cycles of the 91-day extended cycle regimen or 13 consecutive cycles of 28-day (conventional) cycle regimen). In the same study design, patients could also be randomized to a second 91-day extended cycle OC having a lower concentration of ethinyl estradiol (20 μg EE/100 μg LNG) or Levlite® (20 μg EE/100 μg LNG). The intent of the study was to compare the effects of like dosage levels of the 91-day extended cycle regimens to conventional 28-day cycles within the context of separate pair-wise comparisons.

The study was performed in accordance with the Declaration of Helsinki (Republic of South Africa, 1996), applicable guidelines for Good Clinical Practice and with ethics committee approval at each participating clinical site.

Sexually active, adult women (age 18 through 40), of childbearing potential, in a heterosexual relationship, who were at risk for pregnancy, fluent in English, and able to give informed consent were eligible for the study. Active smokers >35 years old were excluded, as were women with any contraindication to the use of OCs, those who had received injectable hormone therapy (e.g., Depo-Provera®) within the 10 months prior to study enrollment, had a progestin-releasing intrauterine device (IUD) in place within three months prior to enrollment, or a contraceptive implant removed within one month prior to enrollment. Routine use of other forms of contraception other than OCs (with the exception of condoms) was also an exclusion to study entry. Those with a recent surgical or medical abortion, miscarriage, or vaginal or cesarean delivery must have had at least two normal menstrual cycles prior to enrollment. Other exclusions included history of abnormal bleeding (breakthrough or withdrawal bleeding that lasts 10 or more consecutive days, or spotting that lasts more than 10 consecutive days) while on conventional OCs, participation in any clinical investigation within 30 days prior to enrollment, and donation or sustained a loss of more than 500 mL of blood within 30 days prior to enrollment. Prohibited medications included use of any medication that might interfere with the efficacy of OCs (e.g., rifampin, barbiturates, antibiotics). At the time of entry into the study, patients were designated as continuous users (those who were on OCs during the cycle prior to entering the study), fresh starts (those with no prior history of OC use), or prior users (those who had a history of OC use in the past without having any OC use in the 6 months prior to enrollment).

Patients could have discontinued from the study for any of the following reasons: any condition that contraindicated the use of OCs, patient decision, pregnancy, any adverse event that made continuation in the study impossible or inadvisable, lost to follow-up, discovered after enrollment not to have met study criteria, refusal to cooperate with required study procedures, or significant lapse of study medication intake (i.e., <80% overall pill taking).

Dosing Regimen and Procedures

Patients randomized to the extended cycle regimens were given blister packs with a 91-day supply of study medication (84 active pills and 7 placebo) at each clinic visit. Four pill packs were dispensed during the one-year study. Patients randomized to the conventional 28-day regimens were supplied with three or four commercial pill packs at each clinic visit, depending on the study month when the next scheduled clinic visit was to take place. All patients received copies of patient instructions for use with each supply of study medication. They were also instructed to return all used pill packs and pill counts were conducted at each clinic visit.

Screening prior to initiation of study therapy and after obtaining informed consent included a medical and contraceptive history, physical examination (including pelvic exam and Pap smear), measurement of vital signs (including weight), clinical laboratory tests (CBC, serum chemistry, lipid profile, and urinalysis), and a urine pregnancy test. Urine pregnancy tests were also obtained at all clinic visits after baseline and at the time of study completion or patient discontinuation.

All patients enrolled in the study completed a daily electronic diary. Questions regarding pill-taking, and occurrence and severity of bleeding/spotting were recorded daily, while responses regarding concomitant contraceptive use and menstrual symptomatology were recorded weekly. All diary entries were time and date stamped to prevent retrospective completion. The diaries were programmed with a reminder alarm in the event more than 24 hours lapsed between diary entries. Patients were provided with paper diaries listing the same questions in the event of electronic diary failure or loss. Concomitant medications and adverse events were recorded separately. At each clinic visit all data from the electronic diaries were downloaded into the investigational site's study database and into a centralized database maintained by the diary vendor.

All patients were to initiate OC therapy on the first Sunday following the beginning of their menstrual period or withdrawal bleed from prior oral contraceptive cycle (“Sunday starters”) and were to remain as Sunday starters throughout the study. They were counseled to take their pill at approximately the same time each day and to record pill intake in the electronic diaries on a daily basis. There were no dosage adjustments, other than in the event that pills were missed.

Patients were seen approximately every three months during the course of the study and at the end of the study. Any patient who withdrew or who was withdrawn from the study had an end-of-study evaluation completed in the same manner as those who completed the full term of study participation. All patients were followed for two months for the occurrence of pregnancy following completion of the study or early withdrawal. Patients who became pregnant were followed for eight weeks following delivery or termination of the pregnancy. Infants were followed for eight weeks following delivery. Compliance with study medication was assessed by daily self-reporting by the patient in electronic or paper diaries. “Compliant use” was defined by eliminating all cycles in which a patient skipped two or more consecutive pills, had a pattern of substantial non-compliance (<80% overall pill-taking), used alternative forms of contraception (including emergency contraceptives), or used prohibited concomitant medications that interact with OC therapy. “Compliant use” patients were also restricted to those patients between the ages of 18 and 35 years, according to the US Food and Drug Administration definition of “optimal” age range for ovulation (see “FDA Guidance for Industry. Combined Oral Contraceptive Labeling for Healthcare Providers and Patients,” U.S. Department of Health and Human Services, Food and Drug Administration, Center for Drug Evaluation and Research (CDER), June 2000 (http://www.fda.gov/cder/guidance/2448dft.pdf).

### Efficacy Assessments

Efficacy was evaluated as the method failure rate, calculated by life table analysis and the Pearl Index (the number of pregnancies per 100 women per year of use) among women age 18-35 who used the product as directed (“compliant use”).

Pregnancy was defined by a positive urine pregnancy test and confirmed by serum human chorionic gonadotrophin (HCG). Conception date was calculated considering all available data such as sonogram data, quantitative HCG, qualitative HCG, pelvic examination, and delivery date. If the conception date was unknown, it was imputed as the midpoint between the patient's last negative pregnancy test date and the date of the positive pregnancy test. It was assumed that the patient was “on study” at the time of conception if the pregnancy occurred between the first and last days of study drug treatment or if the conception date was completely unknown. If conception clearly occurred before the first date of taking study medication, or more than 14 days after the last dose of study drug, it was not counted as a “during study” pregnancy.

Cycle Control Assessment

Cycle control was evaluated by observing the extent of withdrawal bleeding/spotting and breakthrough bleeding/spotting as reported in the electronic diaries. Patients were instructed that bleeding was defined as vaginal blood loss requiring the use of sanitary protection (pads or tampons). Spotting was defined as vaginal blood loss not requiring sanitary protection. All patients responded to questions regarding the presence and intensity of bleeding and/or spotting via a series of preprogrammed questions administered on a daily basis through the electronic diaries.

Bleeding and spotting during each cycle (91 days for the extended cycle regimens and 28 days for conventional regimens), and across the full year of treatment (364 days) were evaluated by assessment of total number of bleeding and/or spotting days, number of “scheduled” bleeding and/or spotting days (i.e., occurring during the 7-day placebo pill interval), and the number of “unscheduled” bleeding and/or spotting days (i.e., occurring during the 84 day active pill interval for the extended cycle regimens or during the 21-day active pill interval for the conventional regimens). Amenorrhea was defined as a lack of withdrawal bleeding during placebo pill intervals.

Evaluation of scheduled bleeding days (i.e., that occurring during the placebo pill interval over the course of one year's treatment) was based on a total of 28 possible days for the extended cycle regimens versus 91 possible days for the conventional regimens.

Evaluation of unscheduled bleeding days (i.e., that occurring during the active pill interval) was based on a total of 336 possible days for the extended cycle regimens versus 273 possible days for the conventional regimens.

Safety Assessment

Safety was evaluated by assessment of self-reported adverse events and adverse events elicited at clinic visits, clinical laboratory tests, vital signs (including weight), and physical examination. All patients who took the study drug were included in the safety assessment. A cohort of patients from the extended cycle regimen groups underwent endometrial biopsy prior to initiation of study medication, and again at the completion of the study to assess the effect of an extended oral contraceptive cycle on the endometrium.

A case report form (CRF) was used to formally record all information regarding reported adverse events. Adverse events were reported spontaneously by the patients, primarily during the regularly scheduled study visit but also by way of the patient's daily diary. For each adverse event, clinical site personnel (a physician or nurse) obtained and recorded additional information pertaining to the seriousness of the adverse event, its severity (mild, moderate, severe, life-threatening), its onset and resolution dates, whether it was still ongoing, the action taken as a result of the event (e.g., no action taken, medical/surgical treatment, interruption of study drug, study discontinuation), and the outcome of the event on the patient's participation in the study (e.g., no effect on participation, study discontinuation, resolution with or without sequalae, death).

Verbatim reported adverse events were classified by body system and preferred term using the MedDRA 4.0 coding nomenclature, a well recognized and standardized system for reporting the incidence and prevalence of adverse events in clinical trials conducted across all therapeutic areas.

Statistical Methods

For this multicenter study, data were pooled across centers. No formal statistical tests were conducted, but descriptive statistics were computed. Life table estimates of the cumulative rate of pregnancy at 52 weeks used 4-week (28-day) intervals for the conventional cycle regimen and 91-day intervals for the extended cycle regimen. Two-sided 95% percent confidence intervals were computed about each cycle's point estimate of the cumulative pregnancy rate. Since the life-table approach is based on a continuous time interval, it includes a patient's entire term of participation in the study, not just completed cycles. The Pearl Index was calculated by dividing the number of on-treatment pregnancies by the total number of complete cycles of exposure (91 days for the extended cycle regimen, and 28 days for the conventional regimen) and expressing the result as an annualized estimate per 100 subjects. The cycles of exposure also included any cycle when a pregnancy occurred.

### Results

Study Population and Disposition

A total of 1394 patients were randomized in a 2:2:1:1 fashion to: (1) the 30 μg EE/150 μg LNG 91-day extended cycle OC (each cycle having 84 days of 30 μg EE/150 μg LNG followed by 7 days of placebo), (2) the 20 μg EE/100 μg LNG 91-day extended cycle OC (each cycle having 84 days of 20 μg EE/100 μg LNG followed by 7 days of placebo), (3) Nordette® conventional regimen (each cycle having 21 days of 30 μg EE/150 μg LNG followed by 7 days of placebo), or (4) Levlite® conventional regimen (each cycle having 21 days of 20 μg EE/100 μg LNG followed by 7 days of placebo) across 47 study sites located throughout the United States. The demographic characteristics of the patients in these treatment groups are presented in Table 2. The four study groups were comparable in terms of racial distribution, mean age, weight, body mass index, smoking status and history of OC use. Over 60% of the patients studied were continuous OC users and an additional 30% had a history of prior OC use but were not on OCs at the time of enrollment (“prior users”). Less than 10% of patients had no history of OC use (“fresh starts”).

Overall, 59.4% (271/456) of the 30 μg EE/150 μg LNG extended cycle regimen patients, 56.2% (260/463) of the 20 μg EE/100 μg LNG extended cycle regimen patients, 71.2% of the 30 μg EE/150 μg LNG conventional regimen patients (161/226), and 68.0% of the 20 μg EE/100 μg LNG conventional regimen patients (157/231) completed one year of therapy on-study. The most common reasons for premature discontinuation were adverse events, individual patient decision, and “lost to follow-up.” The most common adverse events cited as a reason for study discontinuation were bleeding, increased weight, mood swings, and acne. Discontinuation for “unacceptable bleeding” whether cited as an adverse event or as an individual patient decision, accounted for 7.7% of the μg EE/150 μg LNG extended cycle regimen patients, 13.8% of the 20 μg EE/100 μg LNG extended cycle regimen patients, 1.8% of the 30 μg EE/150 μg LNG conventional regimen patients, and 0.9% of the 20 μg EE/100 μg LNG conventional regimen patients. For extended cycle regimen patients, the rate of discontinuation due to unacceptable bleeding decreased considerably after week 26 (i.e., following two extended cycles).

TABLE 2
Demographic characteristics of study patients (N = 1394)

| | | | Extended | |
|---|---|---|---|---|
| | Extended cycle | Conventional | cycle | Conventional |
| | regimen | regimen | regimen | regimen |
| | (30 μg EE/ | (30 μg EE/ | (20 μg EE/ | (20 μg EE/ |
| | 150 μg LNG) | 150 μg LNG) | 100 μg LNG) | 100 μg LNG) |
| Characteristic | (n = 456) | (n = 226) | (n = 463) | (n = 231) |
| Race; n (%) | | | | |
| Caucasian | 351 (77.0) | 169 (74.8) | 361 (78.0) | 171 (74.0) |
| African American | 50 (11.0) | 29 (12.8) | 53 (11.0) | 32 (13.8) |
| Asian | 10 (2.2) | 2 (0.9) | 5 (1.0) | 6 (2.6) |
| Hispanic | 32 (7.0) | 18 (8.0) | 36 (8.0) | 17 (7.4) |
| Other | 13 (2.9) | 8 (3.5) | 8 (2.0) | 5 (2.2) |
| Age (year) | | | | |
| Mean (SD) | 27.8 (5.89) | 27.83 (5.9) | 27.75 (5.93) | 27.36 (5.3) |
| (range) | (18-40) | (19-40) | (18-40) | (18-39) |
| Weight (lb) | | | | |
| Mean (SD) | 156.39 (38.04) | 156.58 (38.7) | 156.32 (39.94) | 153.97 (40.25) |
| (range) | (84-304) | (87-296) | (92-335) | (96-350) |
| Body mass index | | | | |
| (kg/m2) | | | | |
| Mean (SD) | 26.16 (5.9) | 26.31 (6.3) | 26.08 (6.25) | 25.95 (6.41) |
| (range) | (14.47-45.29) | (16.0-47.5) | (15.82-61.4) | (16.23-58.36) |
| Current | | | | |
| smoker? | | | | |
| No (%) | 373 (81.8) | 191 (84.5) | 366 (79.05) | 188 (81.39) |
| Yes (%) | 83 (18.2) | 35 (15.5) | 97 (20.95) | 42 (18.18) |
| OC use history; | | | | |
| n (%) | | | | |
| Unknown | 1 (0.2) | 0 (0.0) | 0 (0.0) | 0 (0.0) |
| Continuous | 288 (63.2) | 142 (62.8) | 277 (59.83) | 139 (60.17) |
| Fresh Start | 35 (7.7) | 14 (6.2) | 36 (7.78) | 21 (9.09) |
| Prior User | 132 (29.0) | 70 (31.0) | 150 (32.40) | 71 (30.74) |
| Compliance | | | | |

There were two measurements of compliance, which were evaluated by assessing patient diary data as to whether or not they took their OC pill every day. Pill compliance within each extended or conventional cycle was determined by observing if the patient missed two consecutive days of pill taking and, if so, the patient was considered to be non-compliant for that cycle. Overall study compliance was determined by counting the percentage of total days in the one-year study when the patient took the designated pill for a given day. Overall compliance of less than 80% would exclude a patient altogether from the Pearl Index calculation. Otherwise, non-compliance within a particular cycle would exclude that cycle only from the Pearl Index. For the life-table calculation, only the overall compliance criterion was used to exclude “non-compliant” patients from the cumulative pregnancy rate calculation, since exclusion of individual cycles from the patient's total would lead to a non-continuous, intermittently truncated time frame.

The overall treatment compliance rate in each of the study groups was very high with 95.4% of the 30 μg EE/150 μg LNG extended cycle regimen patients, 98.1% of the μg EE/100 μg LNG extended cycle regimen patients, 93.4% of the 30 μg EE/150 μg LNG conventional regimen patients, and 96.5% of the 20 μg EE/100 μg LNG conventional regimen patients assessed as compliant. A total of 22 (4.8%) of the 30 μg EE/150 μg LNG extended cycle regimen patients, 16 (3.5%) of the 20 μg EE/100 μg LNG extended cycle regimen patients, 9 (4.0%) of the 30 μg EE/150 μg LNG conventional regimen patients, and 11 (4.8%) of the 20 μg EE/100 μg LNG conventional regimen patients were discontinued from the study due to non-compliance. The number of clinically significant protocol deviations was minimal and no protocol deviations were used to exclude any patients from the analysis of efficacy or safety. Most protocol deviations were related to inclusion/exclusion criteria at study enrollment and were not observed during the active study interval.

Efficacy

Among those patients between the ages of 18 and 35 years, a total of 397 (mean age 26.4 years) received the 30 μg EE/150 μg LNG extended cycle regimen, 408 (mean age 26.4 years) received the 20 μg EE/100 μg LNG extended cycle regimen, 195 (mean age 26.2 years) received the 30 μg EE/150 μg LNG conventional regimen, and 207 (mean age 26.2 years) received the 20 μg EE/1100 μg LNG conventional regimen. During the course of the study, seven (7) patients became pregnant, 4 of 456 (0.9%) treated with the μg EE/150 μg LNG extended cycle regimen, 7 of 463 (1.51%) treated with the 20 μg EE/100 μg LNG extended cycle regimen, 3 of 226 (1.3%) treated with the 30 μg EE/150 μg LNG conventional regimen, and 5 of 231 (2.2%) treated with the 20 μg EE/100 μg LNG conventional regimen. Diary data indicated use of other methods of birth control and/or noncompliance with study medication around the estimated date of conception for three of four of the 30 μg EE/150 μg LNG extended cycle regimen patients, six of seven of the 20 μg EE/100 μg LNG extended cycle regimen patients, one of three of the 30 μg EE/150 μg LNG conventional regimen patients, and one of five of the 20 μg EE/100 μg LNG conventional regimen patients. One additional 20 μg EE/100 μg LNG conventional regimen patient was compliant but was over 35 years of age and, therefore, is excluded from the compliant-use Pearl Index calculation. Thus, one of the 30 μg EE/150 μg LNG extended cycle, one of the 20 μg EE/100 μg LNG extended cycle, two of the 30 μg EE/150 μg LNG conventional cycle, and three of the 20 μg EE/100 g LNG conventional cycle regimen-reported pregnancies were considered method failures. Pearl Index calculations based on method failure were 0.60 for the 30 μg EE/150 μg LNG 91-day extended cycle OC, 0.64 for the 20 μg EE/100 μg LNG 91-day extended cycle OC, 1.78 for the 30 μg EE/150 μg LNG conventional cycle OC, and 2.64 for the 20 μg EE/100 μg LNG conventional cycle OC. Life table point estimates among compliant use patients were 0.55 per 100 women for the 30 μg EE/150 μg LNG 91-day extended cycle OC, 0.65 per 100 women for the 20 μg EE/100 μg LNG 91-day extended cycle OC, 1.45 per 100 women for the 30 μg EE/150 μg LNG conventional cycle OC, and 4.14 per 100 women for the 20 μg EE/100 μg LNG conventional cycle OC. Body weight >90 kg was not a contributing factor in the calculations for the 30 μg EE/150 μg LNG 91-day extended cycle and conventional regimen groups as no patient weighing more than 90 kg became pregnant in those groups. For the 20 μg EE/100 μg LNG 91-day extended cycle group, two non-compliant use patients who became pregnant weighed more than 90 kg.

### Cycle Control

Total Number of Days of Bleeding

The median observed total number of days (out of a possible 364 days) of reported bleeding and/or spotting for all patients enrolled in the study (ITT population) was 35 for the 30 μg EE/150 μg LNG extended cycle regimen versus 53 for the 30 μg EE/150 μg LNG conventional regimen. Among patients treated with the extended cycle regimen, more than half of the total number of days were attributed to spotting. A greater percentage of bleeding only days were reported with the 30 μg EE/150 μg LNG conventional regimen (median 12.2%) and the 20 μg EE/100 μg LNG conventional regimen (median 12.1%) versus the 30 μg EE/150 μg LNG extended cycle regimen (median 5.7%) and the 20 μg EE/100 μg LNG extended cycle regiment (median 7.7%).

Scheduled Withdrawal Bleeding

Due to the differences in the number of cycles of treatment between the 91-day extended cycle treatments and the 28-day conventional regimen (4 vs. 13), and the number of annual hormone free days (28 vs. 91), patients on the extended cycle regimens had fewer total days of scheduled (withdrawal) bleeding/spotting than did patients treated with the conventional regimen. On a per cycle basis, the median number of days of withdrawal bleeding was similar in the four treatment groups. When expressed as a percentage of the total possible days of withdrawal bleeding (28 days for the extended cycle regimens vs. 91 days for the conventional regimens), the median percent of scheduled withdrawal bleeding and/or spotting and bleeding-only days was similar in the four treatment groups.

Unscheduled (Breakthrough) Bleeding

Like all OC products, patients who received the 30 μg EE/150 μg LNG and the 20 μg EE/100 μg LNG extended cycle regimens reported varying degrees of breakthrough bleeding (BTB). The active treatment duration of each extended cycle was four times the length of the active treatment duration for each conventional cycle (84 days vs. 21 days).

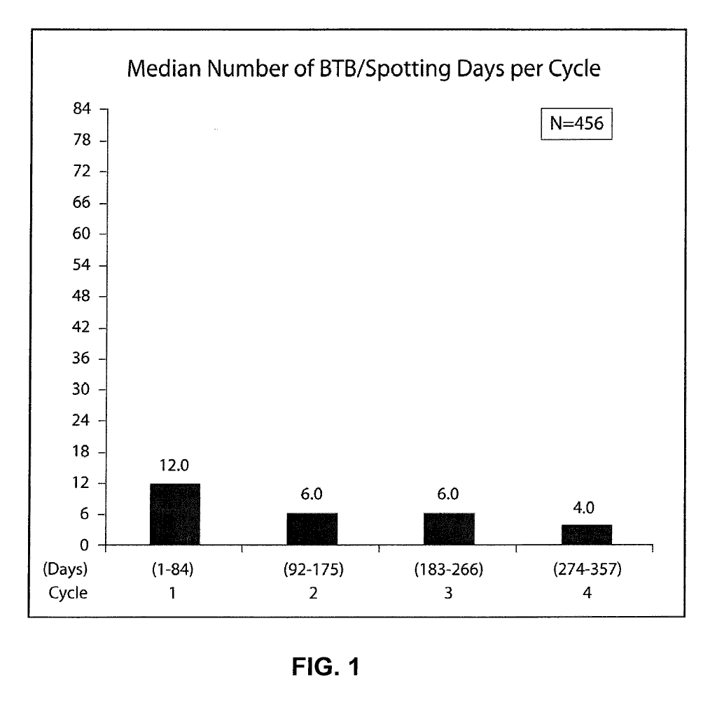

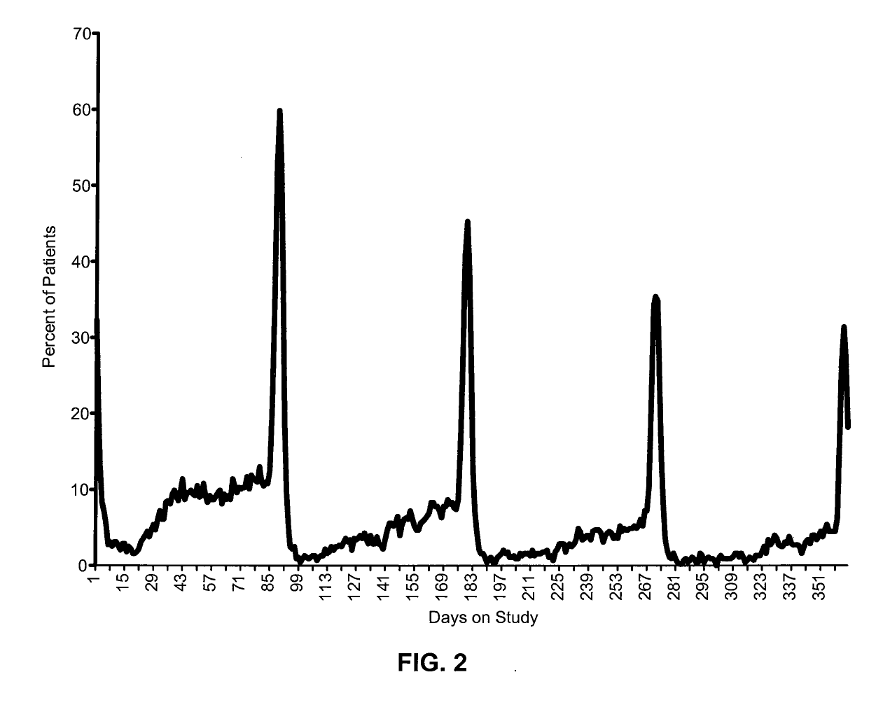

Within the 30 μg EE/150 μg LNG extended cycle regimen treatment group, there were fewer days of BTB with each successive cycle from a median of 12 days during cycle 1 to a median of 4 days during cycle 4. (FIG. 1). Within the 20 μg EE/100 μg LNG extended cycle regimen treatment group, there were fewer days of BTB with each successive cycle from a median of 16 days during cycle 1 to a median of 8 days during cycle 4. The onset of BTB also occurred later within each successive extended cycle and was of shorter duration with each successive extended cycle for the extended cycle regimens. The median number of days of unscheduled bleeding-only days in each cycle, as well as the percentage of patients reporting unscheduled bleeding in each cycle, decreased throughout the course of the study for the extended cycle regimens, as depicted for the 30 μg EE/150 g LNG extended cycle regimen in FIG. 2.

For the 30 μg EE/150 μg LNG extended cycle regimen, patients initially reported slightly more breakthrough bleeding and/or spotting and bleeding only than did patients treated with the conventional regimen. By the last extended cycle (cycle 4), breakthrough bleeding was comparable in the 30 μg EE/150 μg LNG extended cycle regimen and 30 μg EE/150 μg LNG conventional regimen treatment groups. Of the total number of possible days of unscheduled bleeding or spotting days that could be reported (active therapy days: 336 for the 30 μg EE/150 μg LNG extended cycle regimen vs. 273 days for the 30 μg EE/150 μg LNG conventional regimen), a median of 3.6% days on the 30 μg EE/150 μg LNG extended cycle regimen and 2.9% days on the 30 μg EE/150 μg LNG conventional regimen were associated with diary entries of unscheduled bleeding. A median of 6.1% days on the 20 μg EE/100 μg LNG extended cycle regimen and 3.3% days on the 20 μg EE/100 μg LNG conventional regimen were associated with diary entries of unscheduled bleeding.

The majority of patients in the 30 μg EE/150 μg LNG extended cycle regimen and conventional regimen treatment groups reported 5 or less days of unscheduled bleeding per cycle. By the end of the study (cycle 4), 41.5% of the 30 μg EE/150 μg LNG extended cycle regimen patients reported no unscheduled bleeding and more than 80% had 5 days or less. The percentage of patients reporting higher numbers of unscheduled bleeding days (≧6) also decreased with each successive cycle of therapy.

Safety

The incidence rates of adverse events (AEs) were comparable across the treatment groups. AEs reported with the highest incidence rates were those associated with sinus and respiratory tract infections (usually reported by the patient as cold or flu symptoms), headache, and “unexpected” or “breakthrough” bleeding. The incidence of headache was lower for the 30 μg EE/150 μg LNG and 20 μg EE/100 μg LNG extended cycle regimen patients than for the 30 μg EE/150 μg LNG conventional regimen patients (21% and 22% vs. 28%) though similar to the 20 μg EE/100 μg LNG conventional regimen patients (21%). However, higher incidence rates of bleeding-reported events were observed for the 30 μg EE/150 μg LNG and 20 μg EE/100 μg LNG extended cycle regimen patients than for the 30 μg EE/150 μg LNG and 20 μg EE/100 μg LNG conventional regimen patients (12% and 15% vs. 3% and 3%). It should be noted that there was no correlation between “bleeding” reported as an adverse event, “bleeding” reported in the electronic diary, and “bleeding” given as a patient-specified reason for study discontinuation. Shifts in mean laboratory values from baseline to end of study were similar to those reported with other OC therapies (“FDA Guidance for Industry. Combined Oral Contraceptive Labeling for Healthcare Providers and Patients,” U.S. Department of Health and Human Services, Food and Drug Administration, Center for Drug Evaluation and Research (CDER), June 2000 (http://www.fda.gov/cder/guidance/2448dft.pdf)). In addition, the 30 μg EE/150 μg LNG and 20 μg EE/100 μg LNG extended cycle groups reported a lower incidence of pharyngitis (22% and 21% vs. 30%) and upper respiratory infection (URI; 6% and 7% vs. 10%) compared to the 30 μg EE/150 μg LNG conventional regimen though similar values were observed with the 20 μg EE/100 μg LNG conventional regimen (23% for pharyngitis and 6% for URI). A slightly smaller proportion of patients on the 30 μg EE/150 μg LNG and 20 μg EE/100 μg LNG extended cycle regimens reported urinary tract infection (UTI) compared to the 30 μg EE/150 μg LNG and 20 μg EE/100 μg LNG conventional cycle regimens (4% and 5% vs. 6% and 8%). Further, 2% of the 30 μg EE/150 μg LNG and 20 μg EE/100 μg LNG extended cycle regimen patients reported one or more episodes of depression compared to 6% of patients on the 30 μg EE/150 μg LNG conventional regimen, though a similar value of 1% was observed with the 20 μg EE/100 μg LNG conventional regimen.

Changes in triglycerides and LDL cholesterol were comparable between the four treatment groups. There were no clinically meaningful changes in other laboratory values, body weight, vital signs (systolic and diastolic blood pressure, heart rate, or temperature) or in physical exam results from baseline to end of study. There were no reports of endometrial hyperplasia or carcinoma.

Discussion/Conclusions

This was the first large-scale controlled study of extended cycle OC regimens in women up to age 40. The extended cycle regimens consist of a known combination of ethinyl estradiol and levonorgestrel administered at a dose level with a history of proven efficacy and safety. When taken daily, the extended regimen OCs were effective in preventing pregnancy. The adverse event profiles of the extended cycle regimens and the conventional regimen were comparable and similar to those of other oral contraceptives (“FDA Guidance for Industry. Combined Oral Contraceptive Labeling for Healthcare Providers and Patients,” U.S. Department of Health and Human Services, Food and Drug Administration, Center for Drug Evaluation and Research (CDER), June 2000 (http://www.fda.gov/cder/guidance/2448dft.pdf)). The electronic diaries utilized in this study provided a daily “snapshot” of pill compliance and cycle control that is more detailed than any previously published reports.

Noncompliance with pill-taking is particularly troublesome during the transition from one package to the next, an occurrence that takes place 13 times per year with conventional therapy (Adams, H. P. J., “Oral contraception noncompliance: The extent of the problem,” Adv. Contracept. 8(suppl 1):13-20 (1992)). With the use of the extended cycle regimens, the number of transitions between packs is decreased to 4 per year, which can contribute to improved compliance. In this study, compliance with the treatment regimens was very high, possibly due, in part, to daily reminders regarding pill-taking conveyed via use of the electronic diary.

All OCs are associated with unscheduled breakthrough bleeding during the active pill phase. (See “FDA Guidance for Industry. Combined Oral Contraceptive Labeling for Healthcare Providers and Patients,” U.S. Department of Health and Human Services, Food and Drug Administration, Center for Drug Evaluation and Research (CDER), June 2000 (http://www.fda.gov/cder/guidance/2448dft.pdf)). It is also well recognized that breakthrough bleeding diminishes with continued use of OCs. This pattern of reduced incidence of unscheduled bleeding with consecutive cycles of OCs was replicated in this study. Both the median number of days of unscheduled bleeding and/or spotting days as well as the percentage of patients reporting unscheduled bleeding and/or spotting decreased throughout the course of the study in the treatment groups. While the incidence of unscheduled bleeding was higher among patients treated with the extended cycle regimens early in the study, it decreased with each successive cycle of therapy. By the end of the study, the median incidence of unscheduled bleeding reported in the 30 μg EE/150 μg LNG extended cycle regimen group on a patient-monthly basis was comparable to that reported in the conventional regimen group.

Clinical trials and surveys cite bleeding irregularities as the one of the most common reasons for OC discontinuation (Rosenberg, M. J., and Waugh, M. S., “Oral contraceptive discontinuation: a prospective evaluation of frequency and reasons,” Am. J. Obstet. Gynecol. 179:577-82 (1998); WHO Task Force on Oral Contraceptives, “A randomized double-blind study of six combined oral contraceptives,” Contraception 25:231-41 (1982)). Of note, unscheduled bleeding among patients receiving the 30 μg EE/150 μg LNG extended cycle regimen who completed the study was the same as that observed in the ITT population. In this study, 7.7% of the 30 μg EE/150 μg LNG extended cycle regimen patients and 13.8% of the 20 μg EE/100 μg LNG extended cycle regimen patients cited unacceptable bleeding as a reason for discontinuation. It is also notable that the incidence of unscheduled bleeding reported by patients who discontinued for that reason was similar to that reported in women who ultimately completed the study.

Perception of severity of bleeding and acceptance of unscheduled bleeding appeared to be a personal preference. Indeed, the majority of patients rated their overall satisfaction with the extended cycle OC regimen as good to excellent and stated they would choose to have fewer menstrual periods following the completion of the study.

This study demonstrated that extended cycle OCs are effective, safe and well tolerated. The extended cycle regimens represent a change in the paradigm of OC therapy allowing women the option of decreasing the number of withdrawal bleeding intervals from 13 to 4 per year.

### Example 2

A second open-label, non-randomized multicenter study involved extension of the study described in Example 1. In this second study, the extended regimens of Example 1 were administered up to an additional two consecutive years (up to an additional eight 91-day extended cycles) to women desiring pregnancy prevention who had successfully completed one year of therapy in the previous study. Patients who had received the 30 μg EE/150 μg LNG extended cycle or the 30 μg EE/150 μg LNG conventional regimens in the first study were to initially receive the 30 μg EE/150 μg LNG extended cycle regimen in the second study. Patients who had received the 20 μg EE/100 μg LNG extended cycle or the 20 μg EE/100 μg LNG conventional regimens in the first study were to initially receive the 20 μg EE/100 μg LNG extended cycle regimen in the second study.

Investigators had the option, at their discretion, to change the treatment assignment at the start of each new 91-day cycle.

A total of 350 patients were treated; 147 patients discontinued, and 203 completed the study. Patients were evaluated every three months for adverse events, change in smoking history, concomitant medications, study drug compliance, vital signs and pregnancy. Patients completed daily electronic diaries, recording whether they took their pills, providing information on bleeding and spotting and peri-menstrual symptoms. On an annual basis, patients had a physical examination, clinical laboratory examination and a Pap smear.

When used as directed, both extended regimens were >99% effective in preventing pregnancy. Overall rates of study discontinuation and the incidence of adverse events (including serious adverse events and adverse events leading to study discontinuation) were consistent with those reported in the first study described in Example 1. The observed number of patient electronic diary-reported total days, unscheduled days, and scheduled (withdrawal) days of bleeding and/or spotting and bleeding alone were lower than those observed in the one-year study described in Example 1.

Table 3 is a summary of the median days of unscheduled bleeding and/or spotting observed over three years of treatment (with cycles 1-4 representing the first year of treatment described in Example 1 and cycles 5-12 representing the second through third years of treatment described in Example 2). Table 3 shows that while the 20 μg EE/100 μg LNG extended cycle regimen has a more gradual decline in the median number of days of unscheduled bleeding and/or spotting, by the end of the third year of treatment the median number is the same between both extended treatment regimens.

TABLE 3
Unscheduled Bleeding and/or Spotting with Extended Cycle Treatments

| Extended | | | | | | | | | | | | |
|---|---|---|---|---|---|---|---|---|---|---|---|---|
| Cycle | Cycles 1-12 | | | | | | | | | | | |
| Regimens | 1 | 2 | 3 | 4 | 5 | 6 | 7 | 8 | 9 | 10 | 11 | 12 |
| 30 μg EE | (12) | (6) | (6) | (4) | (5) | (2.5) | (2) | (2) | (2) | (2) | (2) | (2) |
| and 150 μg | [3] | [1] | [2] | [1] | [1] | [0] | [0] | [0] | [0] | [0] | [0] | [0] |
| LNG | {9} | {5} | {4} | {3} | {4} | {2.5} | {2} | {2} | {2} | {2} | {2} | {2} |
| 20 μg EE | (16) | (11) | (8) | (8) | (8) | (7) | (6) | (4) | (3) | (3.5) | (2.5) | (2) |
| and 100 μg | [4] | [3] | [2] | [2] | [2] | [1] | [1] | [1] | [1] | [1] | [1] | [0] |
| LNG | {12} | {8} | {6} | {6} | {6} | {6} | {5} | {3} | {2} | {2.5} | {1.5} | {2} |

(median days of unscheduled bleeding and/or spotting)
[median days of unscheduled bleeding]
{median days of unscheduled spotting}

### Example 3

This example provides results of a multicenter, randomized double-blind study evaluating administration of the 20 μg EE/100 μg LNG extended regimen of Examples 1 and 2 following a run-in administration of the 30 μg EE/150 μg LNG extended regimen of Examples 1 and 2.

### Materials and Methods

Study Population

Inclusion criteria were: sexually active women age 18 through 45, at risk for pregnancy, fluent in English, capable of giving and willing to give informed consent, who agreed to routinely use the study OC therapy as their only birth control method, had no contraindication to OCs, and had a history of regular withdrawal or menstrual bleeding for an interval of at least three successive cycles prior to initiation of the study drug. Another birth control method [such as condom, foam or sponge] was required as a back up in situations during the study where two or more pills in a row were missed, where there was a concern about the transmission of sexually transmitted diseases (only a condom could be used in this case), or when intermittent therapies with drugs known to interact with the OCs were initiated.

Exclusion criteria were: active smokers ≧35 years at initiation of the study or who would become 35 during the study; any contraindication to the use of OCs; a history of alcohol or drug abuse, which, in the opinion of the Investigator, made the woman unfit for participation in the study; a history of being HIV or Hepatitis C positive; a history of persistent noncompliance with any chronic medication; a history of having received injectable hormone therapy (e.g., Depo-Provera®) within the 10 months prior to initiation of the study, except Lunelle®, which must not have been taken within 4 months prior to enrollment, or having a progestin-releasing intrauterine device (IUD) in place within one month prior to initiation of the study or having a contraceptive implant removed within one month prior to initiation of the study drug, or having received any other form of hormonal contraception within 3 months prior to initiation of the study drug except oral contraceptives, Nuvaring®, or Ortho-Evra®; concomitant use of additional forms of contraception (IUD, diaphragm, contraceptive sponge, condoms) unless prudent for situations specified in the inclusion criteria, or to prevent sexually transmitted diseases (condom only); recent surgical or medical abortion, miscarriage, or vaginal or cesarean delivery unless at least three consecutive normal menstrual cycles had passed prior to initiation of the study drug; a history of thromboembolic disorder, vascular disease, cerebral vascular or coronary artery disease; a history of uncontrolled or untreated hypertension (systolic BP ≧150 mmHg or diastolic BP ≧90 mmHg); undiagnosed abnormal genital bleeding; a history of hepatic adenomas or carcinomas; a history of cholestatic jaundice of pregnancy or jaundice with prior OC use; known or suspected pregnancy or currently breastfeeding; any clinically significant abnormal finding or condition on history, screening, physical exam, pelvic exam or any laboratory finding which contraindicated the use of OCs; participation in any clinical investigations utilizing investigational drugs or medical devices within the 30 days prior to enrollment; donation or loss of more than 500 cc of blood within the 30 days prior to initiation of the study drug; or abnormality on the screening Pap smear that the Investigator considered clinically significant and believed would interfere with conduct of the study (the Investigator's decision must have been documented).

Patients were to be discontinued from the study if any of the following occurred: the patient demonstrated a pattern of non-compliance that the Investigator believed could not be corrected; the discovery of any condition, which, in the opinion of the Investigator, contraindicated the use of OCs; the patient requested withdrawal from the study; pregnancy; initiation of any medication that might have interfered with the efficacy of OCs such as those described in the exclusion criteria; an adverse event that made patient continuation impossible or inadvisable; the patient was lost to follow-up; the patient refused to cooperate with required study procedures; the patient was ≧35 years old and became a smoker, or smokers who became 35 years old while on the study medication; or the patient had a clinic visit following the final week of a cycle that resulted in a lapse in intake of the study medication.

Study Design

The trial was undertaken to determine how patients would tolerate the lower dose 20 μg EE/100 μg LNG extended cycle regimen administered after an open-label run-in with the 30 μg EE/150 μg LNG extended cycle regimen. Patients received five cycles of therapy, each cycle lasting 91 days, during the course of the study (i.e., Study Months 1-15). Patients received an initial six-month open-label run-in (i.e., Study Months 1-6) of the 30 μg EE/150 μg LNG extended cycle regimen. On completion of the run-in, patients were randomized 2:1 to double-blind treatment for nine-months (i.e., Study Months 7-15) with either (1) the 30 μg EE/150 μg LNG extended cycle regimen, or (2) the 20 μg EE/100 μg LNG extended cycle regimen. Study drug was packaged in blister packs of 91 tablets: 84 active and 7 placebo tablets per pack, and arranged in 13 rows of 7 pills.

Each patient's preference for her assigned regimen was measured at the end of the run-in administration and after 6 months of the double-blind medication (i.e., at Study Month 12). If a patient could not tolerate her initial double-blind treatment assignment she was given the opportunity to cross over to the alternate blinded-treatment at the end of cycle 4 (i.e., at Study Month 12). The Investigators were asked at the end of the run-in and after 6-months of the double-blind treatment whether they would change the patient's OC based on their experience of OC-related side effects.

All patients initiated study OC therapy on the first Sunday following the beginning of their menstrual period and remained Sunday starters throughout the study.

At the screening visit patients had a physical and gynecological exam, Pap smear, clinical laboratory tests (complete blood count (CBC), clinical chemistry, and urinalysis), chlamydia screen, and gave a medical and smoking history. They were given training on how to complete a paper diary and were given a diary to complete between screen and enrollment. After the results from the screening evaluations were known and patient eligibility was confirmed, patients returned to the clinic to receive the initial run-in treatment. At this and at visits every three months, they were queried regarding adverse events, change in smoking history, concomitant medications, and study drug compliance. Vital signs were measured and a urine pregnancy test was done. Paper diaries were reviewed and new diaries were dispensed. At Visit 3 (month 6) patients were randomized in a 2:1 ratio to receive either the 30 μg EE/150 μg LNG or 20 μg EE/100 μg LNG extended cycle regimens. At Visit 3 (month 6), Visit 5 (month 12), and Visit 6 (month 15) patients completed a questionnaire asking how much OC-related symptoms interfered with their daily activities. At Visit 5 (month 12), patients who could not tolerate their treatment assignment were given the option to cross over to the alternate blinded treatment. At the completion of the study or upon discontinuation prior to completion of study therapy, patients had a physical and gynecological examination, clinical laboratory examination, urine pregnancy test and a Pap smear. They were queried regarding adverse events, change in smoking history, concomitant medications, and study drug compliance. Vital signs were measured. Diaries were collected and reviewed. Patients with ongoing serious adverse events were followed until resolution or until the investigator deemed the event to be chronic or stable. Patients who became pregnant during the course of the study were to be followed for eight weeks following delivery or termination of the pregnancy for safety purposes only. Infants were to be evaluated at birth.

Compliance with study medication use was assessed by review of the returned study pill packs and patient diaries at each visit. Non-compliance was defined as all cycles in which a patient skipped two or more consecutive pills, had a pattern of substantial non-compliance with the protocol requirements, used another form of birth control method (BCM) or used a concomitant medication that may interact with OC therapy.

The evaluation of diary based reports of bleeding and spotting during each cycle of treatment was determined by counting the number of bleeding and/or spotting days per cycle for each treatment group. “Unscheduled” (i.e., on active pill days), “withdrawal” (i.e., on placebo pill days), and “total” (active and placebo pill days combined) are reported by cycle.

Efficacy

Efficacy was evaluated from the overall pregnancy rate, calculated by the Pearl Index (the number of pregnancies per 100 women per year of use) using all “On Treatment” pregnancies, defined as those for which the date of conception was on or after the first date of taking study drug but no more than 14 days after the last date of active study drug. No adjustments were made for patients who were crossed over to the alternative treatment group.

Pregnancy was defined by a positive pregnancy test. Conception date was calculated based on the estimated gestational age from an ultrasound report. In situations where ultrasound information was not available to calculate the date of conception, an approximate date of conception was estimated based on the other data that were available (dates of positive pregnancy test, date of last menses, date of birth of the infant, etc.). It was assumed that the patient was on drug at the time of conception if the pregnancy occurred between the first and last days of study drug treatment or if the conception date was completely unknown and could not be reasonably ascertained from any other available data.

Safety Assessment

Adverse events (AEs) reported during the treatment phase of the study served as the principal means to evaluate safety. Each reported event was classified according to the MedDRA system, by organ system classification (body system) and preferred term. The overall incidence of AEs was determined by severity, relationship to treatment, and by time to onset. Information regarding pregnancy, including outcome, in patients with positive pregnancy tests was summarized in the form of patient narratives. No formal statistical analysis was conducted.

### Results

Study Population and Disposition

Table 4 shows the distribution of patients analyzed. A total of 1070 patients were enrolled and treated with the 30 μg EE/150 μg LNG extended cycle regimen during the run-in administration. Of those, 694 (65%) completed the 6-month run-in and were randomized in a 2:1 fashion to either the 30 μg EE/150 μg LNG extended cycle regimen (“30 μg EE/150 μg LNG Randomized”) (N=229) or the 20 μg EE/100 μg LNG extended cycle regimen (“20 μg EE/100 μg LNG Randomized”) (N=465). Fifteen patients discontinued between the screening visit and taking their first dose of study medication: five due to patient personal decision, five became pregnant, one was lost to follow-up, one experienced an adverse event, and three never started the study medication for other reasons (i.e., menses never started). Eight hundred and thirty-four (834-78%) of the treated patients were between the ages of 18 and 35 years (PITT).

TABLE 4
Patients Analyzed

| | 30 μg EE | | 30 μg EE | | 20 μg EE | | | |
|---|---|---|---|---|---|---|---|---|
| | and 150 μg | | and 150 μg | | and 100 μg | | | |
| | LNG | | LNG | | LNG | | | |
| | Run-in Only | | Randomized | | Randomized | | Total | |
| | (N = 376) | | (N = 229) | | (N = 465) | | (N = 1070) | |
| | N | % | N | % | N | % | N | % |
| Treated | 376 | 100.0 | 229 | 100.0 | 465 | 100.0 | 1070 | 100.0 |
| Patients | | | | | | | | |
| Treated | 312 | 83.0 | 170 | 74.2 | 352 | 75.7 | 834 | 77.9 |
| Patients: | | | | | | | | |
| PITT | | | | | | | | |

Of the treated patients who discontinued the study, the majority discontinued the study during the run-in (376 of the 546 that discontinued (69%)). Patients who successfully completed the run-in and were randomized into the double-blind treatment groups had similar discontinuation rates, 26.5% for the 20 μg EE/100 μg LNG regimen and 20.5% for the 30 μg EE/150 μg LNG regimen. Overall discontinuation included: 80 of 1070 patients (7.5%) who withdrew of their own volition during the run-in; 49 of 694 patients (7.1%) who withdrew of their own volition during the double-blind treatment (8.0% for the 20 μg EE/100 μg LNG regimen vs. 5.2% for the 30 μg EE/150 μg LNG regimen); 103 of 1070 patients (9.6%) lost to follow-up during the run-in; and 36 of 694 patients (5.2%) lost to follow-up during the double-blind treatment. Such patients often desired to get pregnant or no longer showed interest in participating in a 15 month study. Slightly higher discontinuation rates were seen when patients over 35 years of age were excluded, suggesting that patients over 35 were more likely to stay in the study for its full duration. Higher percentages of patients reported adverse events as their principal reason for discontinuation during the run-in (13.1%) compared to either the 20 μg EE/100 μg LNG regimen (8.6%) or the 30 μg EE/150 μg LNG regimen (5.2%) during the double-blind phase of the study. Such events were usually associated with “unacceptable bleeding”. Although AEs relating to “unacceptable bleeding” were a common reason for study discontinuation, most patients either discontinued the study for reasons other than “unacceptable bleeding” or remained in the study.

Compliance

Treatment compliance was determined using the pills remaining in the returned pill packs. Patients were not required to record their pill taking; therefore, it was assumed that if the pill was removed from the pill pack that the pill was taken appropriately. If two or more consecutive pills were missed (remaining in the returned pill pack) the patient was deemed non-compliant with the individual cycle. Also, the number of pills returned was compared to the days of exposure as calculated by first and last dose dates to determine the patient's overall compliance with study medication. A high level of overall compliance was observed for patients who successfully completed the run-in and were randomized into the double-blind portion of the study. Well over 90% of all patients took their daily pill at least 80% of the time in both the total treatment group and the PITT cohort. There was no noticeable difference in treatment compliance between the patients randomized to the 30 μg EE/150 μg LNG regimen or the 20 μg EE/100 μg LNG regimen. There was, however, a lower compliance in the non-randomized patients. Patients who discontinued the study during the run-in were less likely to be compliant with study medications (85% took their study medication more than 80% of the time compared with >99% for the randomized patients). This is not surprising since non-compliant patients are more likely to become pregnant, discontinue, or be discontinued by the Investigator.

Efficacy

A total of 28 treated patients became pregnant during the study; 25 of these pregnancies were among patients between the ages of 18 and 35 years (in the PITT cohort). The breakdown of these pregnancies in the PITT cohort included 16/834 (1.9%) patients during the run-in, 7/352 (2.0%) patients randomized to the 20 μg EE/100 μg LNG regimen, and 2/170 (1.2%) patients randomized to the 30 μg EE/150 μg LNG regimen. In addition, there were 10 patients who were screened and became pregnant, but either were never dispensed or never started study medication. Prior users and fresh-starts were advised to use another form of birth control during their first seven days on treatment, therefore any pregnancy in these patients that occurred during this time were considered to be an “Off-Treatment” pregnancy. Thirteen of the 25 pregnancies that occurred in the PITT cohort were considered to be “On-Treatment” pregnancies, 9 were during the run-in and 4 occurred in patients randomized to 20 μg EE/100 μg LNG regimen during the double-blind portion of the study. Only one of the 20 μg EE/100 μg LNG regimen “On-Treatment” pregnancies was considered to be a “compliant use” pregnancy; five of the run-in pregnancies were also considered “compliant-use” pregnancies. Since patients over 35 years of age were not included in the PITT cohort they were not considered in the determination of “On” or “Off” treatment.

Unscheduled (Breakthrough) Bleeding

In looking at the overall pattern of diminishing bleeding and/or spotting, the 30 μg EE/150 μg LNG regimen was associated with a faster reduction in the amount of bleeding and/or spotting compared to the lower dose 20 μg EE/100 μg LNG regimen. Initially after the switch from the 30 μg EE/150 μg LNG regimen to the lower dose 20 μg EE/100 μg LNG regimen (occurring at Cycle 3), the amount of bleeding and/or spotting per cycle increased from 1.8 days at the end of cycle 2 to 2.8 days at the end of cycle 3. However, by the end of cycle 5 (3 cycles after the run-in) the rates of bleeding and/or spotting were only slightly higher for the lower dose 20 μg EE/100 μg LNG regimen at 1.8 days, compared to 1.5 days for the 30 μg EE/150 μg LNG regimen.

Similar results were seen for total unscheduled bleeding and/or spotting. The two randomized treatment groups demonstrated an overall pattern of decreased unscheduled bleeding and/or spotting over time. The overall amount of unscheduled bleeding and/or spotting for the 30 μg EE/150 μg LNG regimen was less than for the 20 μg EE/100 μg LNG regimen. As was seen with total bleeding and/or spotting, there was an initial increase in the amount of bleeding after the switch to the lower dose hormone regimen following the run-in, with the number of unscheduled bleeding and/or spotting days being 2 days at the end of cycle 3 as compared to the 1 day seen for all patients at the end of the run-in (i.e., at the end of cycle 2). Despite the initial increase, by the end of the study (cycle 5) the rates of unscheduled bleeding and/or spotting were similar between the two randomized treatments (30 μg EE/150 μg LNG regimen=0.5 days, lower dose hormone regimen=0.8 days).

Table 4 shows the median days of unscheduled bleeding and/or spotting associated with the extended regimens after an initial run-in with the 30 μg EE/150 μg LNG regimen. Following the run-in, the median days of unscheduled bleeding and/or spotting are similar between the two regimens by the 5^{th} cycle, with patients on the 30 μg EE/150 μg LNG regimen showing a median of 2 days of spotting and patients on the 20 μg EE/100 μg LNG regimen showing a median of 3 days of spotting. Neither regimen produced unscheduled bleeding by the fifth cycle. A corresponding similarity was not observed between the regimens in the study described in Example 2 until later cycles, such as cycle 9. Patients on the 20 μg EE/100 μg LNG regimen in absence of an initial run-in with higher doses of hormones did not experience zero days of unscheduled bleeding until cycle 12 as shown in Table 3, versus cycle 5 following the run-in as shown in Table 5. As such, the data shows that pretreatment with a higher dose extended regimen can lead to a more rapid reduction in the number of days of unscheduled bleeding and/or spotting associated with a lower dose hormone regimen.

TABLE 5
Unscheduled Bleeding and/or Spotting Following a Run-In with a
Higher Dose Regimen

| Extended | | | | | |
|---|---|---|---|---|---|
| Cycle | Cycles 1-5 | | | | |
| Regimens | 1 | 2 | 3 | 4 | 5 |
| 30 μg EE | (9) | (4) | | | |
| and 150 μg | [4] | [0] | | | |
| LNG | {5} | {4} | | | |
| (cycles 1-2) | | | | | |
| 30 μg EE | | | (3) | (2) | (2) |
| and 150 μg | | | [0.5] | [0] | [0] |
| LNG | | | {2.5} | {2} | {2} |
| (cycles 3-5) | | | | | |
| 20 μg EE | | | (8) | (6) | (3) |
| and 100 μg | | | [2.5] | [3] | [0] |
| LNG | | | {5.5} | {3} | {3} |
| (cycles 3-5) | | | | | |

(median days of unscheduled bleeding and/or spotting)
[median days of unscheduled bleeding]
{median days of unscheduled spotting}

### Example 4

A group of women will be administered the 30 μg EE/150 μg LNG extended cycle regimen (84 days of a combination of 30 μg EE and 150 μg LNG, followed by 7 days of placebo) during a run-in of one or more cycles, followed by administration of a lower dose hormone regimen.

Groups of women will be administered the 30 μg EE/150 μg LNG extended cycle regimen for a run-in of 2 cycles. Each group will then receive one of the following lower dose hormone regimens after the run-in: (1) an extended bridged regimen in which a combination of 100 μg levonorgestrel and 20 μg ethinyl estradiol is administered for 84 days followed by 7 days of 10 μg ethinyl estradiol; (2) an extended regimen in which a combination of 100 μg levonorgestrel and 20 μg ethinyl estradiol is administered for 84 days followed by 7 days of placebo; (3) a conventional or commercially available regimen containing a reduced amount of estrogen and/or progestin; or (4) a bridged regimen in which a combination of 150 μg desogestrel and 20 μg ethinyl estradiol is administered for 21 days followed by 7 days of 10 μg ethinyl estradiol. The lower dose hormone regimens will be administered continuously and consecutively for one or more cycles following the run-in period. Certain groups will be administered the lower dose hormone regimen for 3, 4, 5, or 6 cycles following the run-in period.

Each group of women being administered a lower dose hormone regimen after administration of the run-in regimen will experience a reduction in the median number of days of unscheduled bleeding and/or spotting compared to the median number of days of unscheduled bleeding and/or spotting experienced at a corresponding cycle by a group of women being administered the lower dose hormone regimen without having received the initial run-in regimen.

### Example 5

### Multicenter Randomized Phase III Clinical Trial to Evaluate Two Continuous Oral Contraceptive Regimens in Women Diagnosed with Premenstrual Syndrome (PMS) and Premenstrual Dysphoric Disorder (PMDD)

### Clinical Design and Summary

In a multicenter, randomized, clinical trial the efficacy and safety of three combination oral contraceptives regimens in the prevention of pregnancy in sexually active women, ages 18 through 40 years, will be evaluated. Patients will be randomized in a 1:1:1 fashion to one of the following regimens:

Levonorgestrel 150 μg/ethinyl estradiol (EE) 30 μg administered once daily for 84 days as a combination oral tablet followed by ethinyl estradiol 30 μg administered once daily for 7 days (DP3-84/30);

Levonorgestrel 150 μg/ethinyl estradiol 30 μg administered once daily for 84 days as a combination oral tablet followed by ethinyl estradiol 10 μg administered once daily for 7 days (DP3-84/10); or

Levonorgestrel 150 μg/ethinyl estradiol 30 μg administered once daily for 25 days as a combination oral tablet followed by ethinyl estradiol 30 μg administered once daily for 3 days (DP3-25/30).

Patients randomized to either DP3-84/30 or DP3-84/10 will receive 4 cycles of study drug. Patients randomized to DP3-25/30 will receive 13 cycles of study drug. All patients will receive approximately 1 year of therapy.

The study coordinator or designated personnel will register the patient. Patients will be randomly assigned to one of the treatment regimens. The treatment group assignment will not be revealed to the patient prior to signing of the informed consent.

All patients, regardless of randomization, will initiate study OC therapy on the first Sunday following the beginning of their menstrual period (“Sunday starters”) and will remain as Sunday starters throughout the study. Each of the dose packs will be dispensed with an abbreviated patient information sheet and a more detailed patient package insert (PPI).

All patients will complete and download information entered into an electronic diary. Assessments will include study drug compliance, use of additional forms of contraception, bleeding patterns, weight, assessment of the incidence and severity of menstrual related symptoms and medication taken to relieve these symptoms. Information will be self-recorded on the electronic diary via a series of pre-programmed questions.

Two hundred (200) patients in each treatment arm are targeted to complete the study. Pregnancy rate will be calculated using data from those patients age 18 to 35. Patients age 36 through 40 will also be enrolled.

### Patient Eligibility

Inclusion Criteria

Patients must meet the following criteria to be included in the study:

1. Sexually active adult females (age 18 through 40), of child bearing potential, in a heterosexual relationship, at risk for pregnancy, who are in good health and who

have a history of OC use for an interval of at least three successive cycles with regular withdrawal bleeding (bleeding during the pill-free interval or during the first three days of the subsequent cycle) prior to enrollment (Continuous Users)
OR

have no prior history OC use (Fresh-Starts)
OR

have no history of OC use in the 6 months prior to enrollment (Prior Users)

2. Negative urine pregnancy test.

3. Signed informed consent.

4. Agree to use study oral contraceptive therapy as their primary birth control method (BCM).

Exclusion Criteria:

Patients will be excluded from the study if any of the following criteria are met:

1. History of hypersensitivity to estrogen or progestin components of OCs.

2. History of alcohol or drug abuse which, in the opinion of the investigator, makes the patient unfit for participation in the study.

3. Active smoker age >34 years.

4. Chronic use of any medication that may interfere with the efficacy of oral contraceptives.

5. History of being HIV or Hepatitis C positive.

6. History of persistent noncompliance with any chronic medication.

7. History of having received injectable hormone therapy (e.g., Depo-Provera® (Pharmacia and Upjohn)) within the 10 months prior to enrollment or having a progestin-releasing intrauterine device (IUD) in place within 3 months prior to enrollment or has had a contraceptive implant removed within one month prior to enrollment or has received any other form of hormonal contraception within 3 months prior to enrollment.

8. Routine concomitant use of additional forms of contraception (IUD, diaphragm, contraceptive sponge) with the exception of condoms.

9. Patients who have had recent surgical or medical abortion, miscarriage, or vaginal or cesarean delivery must have had at least two normal menstrual cycles prior to enrollment.

10. History of abnormal bleeding (breakthrough or withdrawal bleeding that lasts ≧10 consecutive days or excessive spotting that lasts ≧10 consecutive days) while on conventional oral contraceptives.

11. History of thromboembolic disorder, vascular disease, cerebral vascular or coronary artery disease.

12. Uncontrolled or untreated hypertension (systolic BP ≧140 mmHg and diastolic BP ≧90 mmHg on more than two occasions).

13. Known or suspected carcinoma of the breast, endometrial carcinoma or known or suspected estrogen dependent neoplasia.

14. Undiagnosed abnormal genital bleeding.

15. History of hepatic adenomas or carcinomas.

16. History of cholestatic jaundice of pregnancy or jaundice with prior OC use.

17. Known or suspected pregnancy or currently breastfeeding.

18. Hyperlipidemia requiring active treatment with antihyperlipidemic agents.

19. History of diabetes mellitus, glucose intolerance or gestational diabetes.

20. History of abnormal laboratory value at screening

21. Any clinically significant abnormal finding or condition on history, screening, physical exam, pelvic exam or any laboratory finding which contraindicates the use of oral contraceptives.

22. Has participated in any clinical investigation within the 30 days prior to enrollment.

23. Has donated or had a loss of more than 500 cc of blood within the 30 days prior to enrollment.

### Treatment Regimen

Description of Study Medication

DP3-84/30

All tablets in the DP3-84/30 regimen; 84 tablets each containing 150 μg levonorgestrel/30 μg EE and 7 tablets each containing 30 μg of EE will be white unembossed tablets. One combination tablet will be taken each day for 84 days followed by 7 days of EE tablets in 91-day cycles repeated consecutively for approximately one year (4 cycles). Each DP3-84/30 dose kit will be packaged in a 3-part fold-out white blister card pack where each of the first two blister packs has 28 active tablets each and the third blister pack has 28 active tablets and 7 ethinyl estradiol tablets (35 tablets total) for each 91-day cycle.

Each blister card pack will be sealed into a foil pouch, which will be labeled with a patient-specific label. Each foil pouch will contain an oxygen absorber. At each clinic visit one foil pouch, a patient information sheet, a PPI and a child resistant pouch will be dispensed.

DP3-84/10

All tablets in the DP3-84/10 regimen; 84 tablets each containing 150-μg levonorgestrel/30-μg EE and 7 tablets each containing 10 μg of EE will be white unembossed tablets. One combination tablet will be taken each day for 84 days followed by 7 days of EE tablets in 91-day cycles repeated consecutively for approximately one year (4 cycles). Each DP3-84/10 dose kit will be packaged in a 3-part fold-out white blister card pack where each of the first two blister packs has 28 active tablets each and the third blister pack has 28 active tablets and 7 ethinyl estradiol tablets (35 tablets total) for each 91-day cycle.

Each blister card pack will be sealed into a foil pouch, which will be labeled with a patient-specific label. Each foil pouch will contain an oxygen absorber. At each clinic visit one foil pouch, a patient information sheet, a PPI and a child resistant pouch will be dispensed.

DP3-25/30

All tablets in the DP3-25/30 regimen; 25 tablets each containing 150-μg levonorgestrel/30-μg EE and 3 tablets each containing 30 μg of EE will be white unembossed tablets. One combination tablet will be taken each day for 25 days followed by 3 days of EE tablets in 28-day cycles repeated consecutively for approximately one year (13 cycles). Each DP3-25/30 blister card will have 25 active tablets followed by 3 ethinyl estradiol tablets (28 tablets total) for each 28-day cycle.

Each blister card will be sealed into a foil pouch, which will be labeled with a patient-specific label. Each foil pouch will contain an oxygen absorber. At clinic visits one through three, 3 foil pouches, a patient information sheet, a PPI and a child resistant pouch will be dispensed. At clinic visit four, 4 foil pouches, a patient information sheet, a PPI and a child resistant pouch will be dispensed.

All patients, regardless of randomization, will be instructed to initiate OC therapy on the first Sunday following the beginning of their menstrual period (“Sunday starters”). Patients will be instructed to take their study medication at the same time each day. Day 1 of the study will be defined as the first day of study medication.

Administration

Designated personnel will dispense all study drugs. All study medications must be kept in a secured area at temperature ranging from approximately 15-25° C. (59-77° F.). All patients will be instructed to take one tablet per day at approximately the same time each day. All patients will be “Sunday starters”; that is all patients will begin study drug therapy on the first Sunday following the start of their previous menstrual cycle or completion of prior oral contraceptive regimens. All patients enrolled in the study will maintain Sunday starts for each successive cycle.

The end-of-study evaluation will take place 1 week following completion of withdrawal menses following the last cycle of study OC therapy. At the clinic visit during which patients receive the final supply of study medication, they will be counseled to use an alternative method of birth control during the interval between when they have finished study medication until they have completed the final study visit.

Patients randomized to DP3-84/30 or DP3-84/10 will receive a 13-week supply (single cycle) of study drug at each clinic visit during Weeks 13, 26 and 39. Patients randomized to DP3-25/30 will receive a 12-week supply (three-cycles) of study drug at the initiation of the study and at clinic visits during Weeks 12 and 24. During the clinic visit at Week 36 patients randomized to DP3-25/30 will receive 16-week supply (four cycles) of study medication.

### Examinations/Tests

TABLE 6
Study Schedule

| | | | Visits | Completion of |
|---|---|---|---|---|
| Parameter | Screening | Visit 1 | 2-4^{a} | Therapy |
| Informed consent | X | | | |
| Medical and contraceptive | X | | | |
| history | | | | |
| Physical exam including | X | | | X |
| pelvic exam | | | | |
| Weight, vital signs | X | X | X | X |
| Pap smear | X | | | X |
| Randomization | | X | | |
| Clinical laboratory tests^{b} | X | | | X |
| Urine pregnancy test^{c} | X | X | X | X |
| Study drug distribution^{d} | | X | X | |
| Electronic diary distribution | | X | | |
| Study drug compliance | | | X | X |
| measurement | | | | |
| Adverse event recording | | | X | X |

^{a}Patients randomized to DP3-84/30 or DP3-84/10 will be seen at Weeks 13, 26 and 39. Patients randomized to DP3-25/30 will be seen at Weeks 12, 24, and 40.
^{b}Clinical laboratory tests include CBC, serum chemistry, lipid profile, urinalysis
^{c}Repeated on Visit 1 if the screening was completed more than 2 weeks prior to enrollment
^{d}For patients randomized to DP3 25/30, three (3) cycle supply will be dispensed at Weeks 12 and 24; a four (4) cycle supply will be dispensed at Week 40.

### Study Procedures by Visit

Screening and Enrollment

Patients will sign informed consent. Prior to enrollment, within four weeks prior to initiation of study therapy, all patients will undergo a screening evaluation that will include prior medical and contraceptive history, smoking history, physical examination including pelvic exam and Pap smear, vital signs and weight, and clinical laboratory tests including complete blood count (CBC), serum chemistry, lipid profile, urinalysis, and urine pregnancy test.

All clinical laboratory evaluations (blood and urine) will be tested by a central laboratory. All investigators will be provided with a laboratory manual that outlines sampling and shipping procedures.

If the screening evaluation is completed more than two weeks prior to the initiation of study therapy, the urine pregnancy test must be repeated at Visit 1. Patients with a report of an abnormality on Pap smear will be disqualified for enrollment unless investigator decides the results are not clinically significant and will not interfere with conduct of the study. Investigator's decision must be documented. Patients who have had a normal Pap smear within the three months prior to enrollment in the study will not be required to have the test repeated. A copy of the results must be available in the patient's medical record. Any patient with a report of insufficient cells must have the test repeated and documented as normal prior to enrollment. Patients will then be enrolled in the study.

Visit 1

Visit 1 will take place during the final week of the menstrual cycle prior to beginning study therapy (i.e., during menses for those patients not taking oral contraceptives or during Week 4 for those patients taking oral contraceptives). During Visit 1 patients will be randomized to one of the following treatment groups:

DP3-84/30; levonorgestrel 150 μg/EE 30 μg for 84 days+EE 30 μg for 7 days
OR

DP3-84/10; levonorgestrel 150 g/EE 30 μg for 84 days+BE 10 μg for 7 days
OR

DP3-25/3; levonorgestrel 150 μg/EE 30 μg for 25 days+EE 30 μg for 3 days

The treatment regimen assignment will be ascertained by randomization via Interactive Voice Response System (IVRS). The treatment group assignment will not be revealed to the patient prior to signing of the informed consent.

A urine pregnancy test will be re-administered to those women who were screened more than two weeks prior to Visit 1. Study medication will be dispensed with patient instructions. An electronic diary will be given to each patient. Each patient will be trained regarding the use and care of the electronic diary. Patients will be instructed to take each dose of study medication and to complete all diary entries at approximately the same time each day.

Visits 2-4

All visits should take place within seven days prior to completion of study medication for that cycle. Any visit that takes place prior to the final week of the cycle will be recorded as a protocol deviation. Any visit that takes place following the final week of the cycle resulting in a lapse in study medication intake will be recorded as a protocol violation and will result in the patient being withdrawn from the study. Any visit that takes place following the final week of the cycle but does not result in a lapse in study medication (e.g., the patient received an emergency supply of study medication) will be recorded as a protocol deviation.

Patients randomized to either DP3-84/30 or DP3-84/10 will be seen at Weeks 13, 26 and 39. Patients randomized to DP3-25/30 will be seen at Weeks 12, 24 and 36. During these visits, patients will be queried regarding adverse events, concomitant medications, change in smoking history, and compliance. Vital signs and weight will be recorded. A urine pregnancy test will be conducted. Used study medication will be returned and counted by the study pharmacist or designated personnel.

Completion of Therapy

The end-of-study evaluation will take place 1 week following completion of last cycle of the study drug. Patients will be counseled to use birth control during the interval between when they have finished study medication until they have completed the final study visit. Patients will undergo physical exam, including pelvic exam and pap smear. Vital signs and weight will be recorded. Blood and urine samples for clinical laboratory tests including CBC, serum chemistry, lipid profile, urinalysis and urine pregnancy test will be obtained. Used study medication cards will be returned and counted by the study pharmacist or designated personnel. Patients will be queried regarding adverse events, concomitant medications, change in smoking history and compliance. The electronic diary will be returned.

Post-Study Visit

After study completion/withdrawal, patients will be followed via a phone call for occurrence of pregnancy and until the menstrual cycle returns to normal. The patient based on the cycle pattern prior to the study entry will determine return to normal menstrual cycle. The minimum period of follow up will be 3 months. Patients who decide to use a contraceptive method that regulates/alters menstrual cycle after study completion/withdrawal will be followed for 3 months via a phone call.

Only those patients who have an on-going serious adverse event that has not resolved or those who become pregnant during the course of the study will be followed via clinic visits after completion of the study. Patients with on-going serious adverse events will be followed until the event has been satisfactory managed or resolved. Patients who are pregnant will be followed for eight weeks following delivery or termination of the pregnancy. Infants' health assessment will be followed for eight weeks following delivery. This follow-up may be in the form of a written report from a family physician, obstetrician or pediatrician. All serious adverse events that occur in the three months following discontinuation of therapy will be reported. SAEs that occur at any time after study completion/discontinuation will be reported if investigator determines it is drug-related.

Early Termination

Any patient who withdraws or is withdrawn from the study must return the investigational medication and electronic diary and will be required to complete all procedures for the final visit. All patients will be followed via a phone call for 3 months for the occurrence of pregnancy and until the menstrual cycle return to normal. All patients will be followed via a phone call for three months for the occurrence of serious adverse events.

### Examinations and Procedures

Physical Exam, Medical and Gynecologic History

A complete physical and gynecologic exam, including PAP smear, will be performed at screening and at the completion of therapy or upon early withdrawal from the study. Any patient with an abnormal Pap smear will be disqualified for enrollment unless investigator decides the results are not clinically significant and will not interfere with conduct of the study. The Investigator's decision must be documented. Patients who have had a Pap smear reported as within normal limits within the three months prior to enrollment in the study will not be required to have the test repeated. A copy of the results must be available in the patient's medical record. Any patient with a report of insufficient cells must have the test repeated and documented by the investigator as within normal limits prior to enrollment.

Laboratory Safety Tests

Clinical laboratory tests will be performed at screening and at the completion of therapy or upon early withdrawal. All clinical laboratory tests will be done at one central laboratory. Laboratory tests will include CBC, serum chemistry, lipid profile, urinalysis, and urine pregnancy test. In addition, urine pregnancy tests will be conducted at every clinic visit and at the completion of therapy or upon early withdrawal from the study. All urine pregnancy tests will be performed using the Sure Step® Pregnancy Test kit (Applied Biotech, Inc.).

Pregnancy

All patients will be followed for the occurrence of pregnancy for three months following completion of the study. This follow-up may be in the form of a telephone call.

All pregnancies that occur during the course of the study or in the three months following completion of the study will be dated using ultrasound to establish the gestational age of the fetus. Patients who become pregnant during the course of the study due to method failure will be followed for eight weeks following delivery or termination of the pregnancy. Infants' health assessment will be followed for eight weeks following delivery. This follow-up may in the form of a documented telephone conversation with associated pediatrician or written report from the associated pediatrician.

### Electronic Diaries

Patients will be asked to complete electronic diaries. The diary will be programmed to ask specific questions related to the study compliance, bleeding pattern and occurrence of symptoms that are commonly associated with the hormone fluctuation during the menstrual cycle. The questions will address dosage, compliance, bleeding pattern and hormone-related symptoms either on the scale from 0-3 or using 10 cm Visual Analogue Scale (VAS).

Hand-held data acquisition devices will be used to collect patient responses. The electronic diary will provide patients with a menu-driven, graphical interface to enter diary information (as well as objective data) using a hand-held stylus. Data entry will be electronic and key fields must be completed properly before allowing patient to finish the report. Each report will be downloaded by dial-up network connection.

The electronic diary will incorporate an alarm to remind the patient when to complete their reports. Alarm times will be set by the site and can be specific to the patient preference. The patient will be instructed to complete a diary on a daily basis. Retrospective data entry will not be allowed; reports cannot be completed for previous days. Once each question is completed the patient will confirm the response and will not be permitted to return to that question for modification.

Information on the hormone-related symptoms to be collected is from the Calendar of Premenstrual Experiences (COPE) and Diagnostic and Statistical Manual of Mental Disorders Forth Edition (DSM-IV).

The validity and reliability of the COPE instrument was assessed by Mortola, et al., Obstet. Gynecol. 89:179-83 (1990), who administered it throughout two consecutive ovulatory cycles to 36 rigidly screened women with PMS and to 18 controls. The validity of the visual analogue scales applied to the psychological symptoms associated with the PMDD has been previously documented.

### Treatment Modifications Based on Toxicity

No significant toxicity is expected from the study medication. However, if the patient develops any symptoms or any abnormal laboratory parameter attributed to the drug, which are considered by the patient and/or physician to be of unacceptable severity, then the study medication should be discontinued.

### Concomitant Medications

Patients will be queried regarding concomitant medication use at monthly phone calls and quarterly clinic visits. All concomitant medication use (both prescription and over-the-counter (OTC), including herbal medications and nutritional supplements) must be reported during the study, and recorded on the patient's Case Report Form (CRF).

Patients who require the initiation of chronic therapy with drugs that are known to interact with OCs will be withdrawn from the study. Patients who require intermittent therapy with drugs known to interact with OCs (e.g. antibiotic therapy) will remain in the study and will receive counseling regarding the need for additional contraceptive protection during the entire cycle. Patients will be provided with the list of medications that are know to interact with OC and will be instructed to notify study coordinator as soon as medication is prescribed to receive proper counseling. Notification and counseling can be conducted via the phone and must be documented in the patient's CRF. Those cycles in which drugs known to interact with OC therapy are taken will not be used in the calculation of the pregnancy rate.

The use of emergency contraceptive pills (“morning after pills”) is prohibited in the study. Data from any patient who utilizes contraceptive pills others than those provided for the study will not be included in the calculation of the pregnancy rate for that cycle.

### Adverse Event Reporting

An Adverse Event (AE) is any reaction, side effect, or other undesirable event that occurs in conjunction with the use of a drug, biological product or diagnostic agent in humans, whether or not the event is considered drug related.

A serious adverse event (SAE) is one that meets any one of the following criteria:

Fatal or life threatening

Requires or prolongs inpatient hospitalization

Results in persistent or significant disability/incapacity

Congenital anomaly

The term “life threatening” in the definition of “serious” refers to an event in which the patient was at risk of death at the time of the event; it does not refer to an event that hypothetically might have caused death if it were more severe. Medical and scientific judgment should be exercised in deciding whether an important medical event is serious. Although the event may not be immediately life threatening, fatal, or result in hospitalization, it should be considered serious when it jeopardizes the patient, or requires an intervention to prevent a serious outcome as defined above.

The AE reporting period for this study begins at the Enrollment Visit and ends at the final clinic visit. The SAE reporting period will continue for 3 month after the final clinic visit. All SAEs will be followed through resolution or until investigator assesses the SAE as chronic or stable.

A preexisting condition (i.e., a disorder present before the AE reporting period started and noted on the pretreatment medical history/physical form) should not be reported as an AE unless the condition worsens or episodes increase in frequency during the AE reporting period.

During the study AEs will be recorded through monthly phone calls and quarterly clinic visits. A call-in number will be provided to the patients who wish to report an adverse event between the scheduled phone calls and clinic visits.

### Example 6

### Multicenter Randomized Phase III Clinical Trial to Evaluate Two Continuous Oral Contraceptive Regimens in Combination with Fluoxetine Hydrochloride in Women Diagnosed with Premenstrual Syndrome (PMS) and Premenstrual Dysphoric Disorder (PMDD)

### Overview of the Study Design

In a three-arm, parallel, randomized, multicenter, placebo-controlled, double-blinded study, the efficacy and safety of continuous oral contraceptive therapy as a ninety-one day regimen (84 days active combination therapy followed by low dose estrogen for 7 consecutive days (DP3-91)), or as a twenty-eight day regimen (21 day active combination therapy followed by low dose estrogen for 7 consecutive days (DP3-28)), in combination with fluoxetine hydrochloride administered for approximately 6 consecutive months to women diagnosed with PMS and/or PMDD who desire contraception, will be evaluated.

A cohort of approximate 40-100 patients enrolled in each of the study arms will undergo endometrial biopsy (to test incidence of hyperplasia and carcinoma) prior to the initiation of study drug therapy and at the conclusion of the study or withdrawal. Efficacy of the 28-day and 91-day regimens on premenstrual symptomatology will be measured by psychometric scales that include self-administered Visual Analogue Scales (VAS) and a prospective daily symptoms chart to evaluate psychological and somatic symptoms. The VAS measures tension, irritability, dysphoria, sleeping and eating patterns, headache, bloating, pain and breast tenderness and weight gain symptoms. Total score of the psychological and somatic symptoms will be computed. The patient and blind observer will also complete the PMTS at each visit.

### Study Population

Females ages 18 through 49 who are fluent in English and capable of giving informed consent, without contraindication to the use of oral contraceptives and selective serotonin reuptake inhibitors (SSRIs), and meet the criteria for PMS including PMDD as defined in the diagnostic and statistical manual of mental disorders (DSM-IV). All patients will be counseled at the beginning of the study and at each study visit to use an alternative form of contraception. All patients will be followed for the occurrence of pregnancy during the course of the study. Patients who become pregnant during the course of the study will be followed for eight weeks following delivery or termination of the pregnancy. Infants will be followed for eight weeks following delivery.

### Dosage

Patients will be randomized to one of the following:

(1) Ninety-one day oral contraceptive therapy with ethinyl estradiol (DP3-91) and fluoxetine hydrochloride administered for two cycles where each cycle consists of: 150 μg levonorgestrel and 30 μg ethinyl estradiol (days 1-84 of the first cycle and days 92-175 of the second cycle, 30 μg ethinyl estradiol (days 85-91 of the first cycle and days 176-182 of the second cycle), 20 mg fluoxetine hydrochloride (days 1-182), and placebo to preserve blinding (days 183-196);

(2) Twenty-eight day oral contraceptive therapy with ethinyl estradiol (DP3-28) administered for 7 cycles where each cycle consists of: 150 μg levonorgestrel and 30 μg ethinyl estradiol (days 1-21 for seven cycles), 30 μg ethinyl estradiol (days 22-28 for seven cycles), and 20 mg fluoxetine hydrochloride (days 1-196); or

(3) Fluoxetine hydrochloride administered daily for 196 days: 20 mg fluoxetine hydrochloride per day (days 1-196) or placebo to preserve blinding (days 1-196).

### Study Management

The study will utilize electronic case report forms and remote system management. Each investigator will be provided a programmed laptop computer dedicated to the study. This system allows the investigator to download and view patient diary data during clinic visits and also allows for rapid data queries by the study monitors. The system will also allow real-time on-line tracking of study site accrual rates, serious adverse events, pregnancies and study progress.

### Outcomes Measurement Scales

The primary outcome will be defined as reduction in symptoms of PMS including PMDD as measured by the mean scores on Visual Analogue Scales (VAS) and the Premenstrual Tension Syndrome Scale (PMTS). The VAS will measure tension, irritability, dysphoria, sleeping and eating patterns, headache, bloating, pain and breast tenderness symptoms. Patients will be prompted to rate how they feel each day using 100 mm scales in which the descriptors range from “no symptoms” (0 mm) to “severe or extreme symptoms” (100 mm). The PMTS consists of a 36 item scale that will be completed by the patient and a 10-item scale completed by the blinded observer. Both scales rate premenstrual symptoms for a particular day; the total score can range from 0 (no symptoms) to 36 (all symptoms present and severe).

The secondary outcome will be defined as reduction in symptoms of PMS including PMDD as measured by the sub-score of somatic symptoms on VAS. The VAS will measure headache, bloating, pain and breast tenderness and weigh gain symptoms. Patients will be prompted to rate how they feel each day using 100 mm scales in which the descriptors range from “no symptoms” (0 mm) to “severe symptoms” (100 mm). In addition to information recorded in paper diaries, a standardized questionnaire will be used to determine whether the patient had any side effects.

### Statistical Analysis

For the primary analysis, the mean of the VAS scales will be derived to obtain a single VAS score, which evaluates composite psychological and symptomatic outcomes. Mean percent reduction from baseline at the luteal phase will be compared using an analysis of covariance (ANCOVA) approach that evaluates the effects of the treatment group, center and treatment-by-center interaction, after adjusting for the effect of the baseline VAS score. All statistical tests will be two-sided at the 0.05 level of significance. Pairwise comparisons will be made for each active treatment to placebo. Secondary analyses will include a set of statistical tests for the PMTS and 10-item blinded observer-based measures.

### Example 7

Two multicenter, randomized, Phase III clinical trial studies were conducted following a protocol similar to the protocol presented in Example 5. During both clinical studies, the number of days of bleeding (withdrawal menses and unscheduled bleeding) was monitored.

For the first clinical study, patients were randomized to one of the following oral contraceptive regimens:

Levonorgestrel 150 μg/ethinyl estradiol (EE) 30 μg administered once daily for 84 days as a combination oral tablet followed by ethinyl estradiol 30 μg administered once daily for 7 days (DP3-84/30);

Levonorgestrel 150 μg/ethinyl estradiol 30 μg administered once daily for 84 days as a combination oral tablet followed by ethinyl estradiol 10 μg administered once daily for 7 days (DP3-84/10); or

Levonorgestrel 150 μg/ethinyl estradiol 30 μg administered once daily for 25 days as a combination oral tablet followed by ethinyl estradiol 30 μg administered once daily for 3 days (DP3-25/30).

For the second clinical study, patients were randomized to one of the following oral contraceptive regimens:

Levonorgestrel 150 μg/ethinyl estradiol (EE) 30 μg administered once daily for 84 days as a combination oral tablet followed by ethinyl estradiol 30 μg administered once daily for 7 days (DP3-84/30);

Levonorgestrel 150 μg/ethinyl estradiol 30 μg administered once daily for 84 days as a combination oral tablet followed by ethinyl estradiol 10 μg administered once daily for 7 days (DP3-84/10); or

Levonorgestrel 150 μg/ethinyl estradiol 30 μg administered once daily for 25 days as a combination oral tablet followed by ethinyl estradiol 30 μg administered once daily for 3 days (DP3-25/30); or

Levonorgestrel 150 μg/ethinyl estradiol (EE) 30 μg administered once daily for 21 days as a combination oral tablet followed by 7 days of placebo (conventional 28-day oral contraceptive regimen (“Nordette”)).

Patients randomized to either the DP3-84/30 or DP3-84/10 regimens in both clinical studies received four 91-day cycles of therapy (the designated regimen). Patients randomized to the DP3-25/30 regimen for both clinical studies, or the conventional 28-day regimen in the second clinical study, received thirteen 28-day cycles of therapy. All patients received approximately 1 year of therapy.

Tables 7 and 8 summarize the average number of days of breakthrough bleeding (defined as unscheduled bleeding and/or spotting) per cycle by treatment group, for the first and second clinical studies. The median (per monthly cycle) values in each table represent the median number of days of breakthrough bleeding per 91-day cycle converted to a 28-day cycle

$$( median ⋆ \frac{21}{84} or \frac{21}{25} ,$$

depending on the number of days of combination therapy) for comparison with “Nordette” (conventional 28-day regimen in the second clinical study). Table 7 also presents data from a third Phase III clinical study (Anderson, F. D., et al., Contraception 68:89-96 (2003)) that was conducted following a protocol similar to the protocol presented in Example 6, but in which the patients were randomized to one of the following four regimens:
Levonorgestrel 150 μg/ethinyl estradiol (EE) 30 μg administered once daily for 21 days as a combination oral tablet followed by 7 days of placebo (“Nordette”); or
Levonorgestrel 100 μg/ethinyl estradiol 20 μg administered once daily for 21 days as a combination oral tablet followed by 7 days of placebo; or
Levonorgestrel 150 μg/ethinyl estradiol 30 μg administered once daily for 84 days as a combination oral tablet followed by 7 days of placebo (“Seasonale”); or
Levonorgestrel 100 μg/ethinyl estradiol (EE) 20 μg administered once daily for 84 days as a combination oral tablet followed by 7 days of placebo.
   The Seasonale and Nordette data from this third clinical study are presented in Table 7. All data presented in Table 8 were from the second clinical study.

TABLE 7
Number of Days of Unscheduled Bleeding and/or Spotting Per Cycle, By
Treatment Groups - Complete Cycles Only

| Regimen | | | | | | | | | | Median |
|---|---|---|---|---|---|---|---|---|---|---|
| (Treatment | | | | | | | | | | Per Monthly |
| Group)* | Cycle | N | Mean | SD | Min | Q1 | Median | Q3 | Max | Cycle |
| DP3-84/30 | 1 | 751 | 15.4 | 14.55 | 0 | 4 | 11 | 24 | 74 | 2.8 |
| | 2 | 609 | 12.5 | 13.38 | 0 | 3 | 7 | 20 | 82 | 1.8 |
| | 3 | 515 | 10.0 | 11.12 | 0 | 2 | 7 | 14 | 62 | 1.8 |
| | 4 | 420 | 9.5 | 11.17 | 0 | 2 | 5 | 13 | 68 | 1.3 |
| DP3-84/10 | 1 | 769 | 14.3 | 13.52 | 0 | 3 | 11 | 21 | 73 | 2.8 |
| | 2 | 625 | 9.5 | 10.51 | 0 | 2 | 5 | 14 | 63 | 1.3 |
| | 3 | 531 | 7.3 | 9.12 | 0 | 1 | 4 | 10 | 50 | 1.0 |
| | 4 | 443 | 7.5 | 9.33 | 0 | 1 | 4 | 10 | 58 | 1.0 |
| Seasonale | 1 | 385 | 16.4 | 15.0 | 0 | 3 | 14 | 25 | 84 | 3.5 |
| | 2 | 331 | 12.3 | 13.0 | 0 | 2 | 7 | 19 | 66 | 1.8 |
| | 3 | 296 | 10.8 | 12.4 | 0 | 1 | 6 | 15.5 | 62 | 1.5 |
| | 4 | 262 | 9.1 | 11.0 | 0 | 1 | 4 | 15 | 55 | 1.0 |
| Nordette | 1 | 214 | 2.1 | 3.1 | 0 | 0 | 1 | 3 | 19 | |
| | 2 | 210 | 1.9 | 2.2 | 0 | 0 | 1 | 3 | 12 | |
| | 3 | 204 | 1.7 | 2.3 | 0 | 0 | 1 | 2 | 12 | |
| | 4 | 194 | 1.3 | 1.8 | 0 | 0 | 1 | 2 | 14 | |
| | 5 | 188 | 1.6 | 2.0 | 0 | 0 | 1 | 2 | 11 | |
| | 6 | 184 | 1.5 | 1.9 | 0 | 0 | 1 | 2 | 14 | |
| | 7 | 178 | 1.4 | 1.6 | 0 | 0 | 1 | 2 | 8 | |
| | 8 | 177 | 1.6 | 2.0 | 0 | 0 | 1 | 2 | 14 | |
| | 9 | 172 | 1.6 | 2.1 | 0 | 0 | 1 | 2 | 16 | |
| | 10 | 170 | 1.7 | 2.3 | 0 | 0 | 1 | 3 | 20 | |
| | 11 | 163 | 2.0 | 2.6 | 0 | 0 | 1 | 3 | 19 | |
| | 12 | 162 | 1.6 | 2.0 | 0 | 0 | 1 | 3 | 11 | |
| | 13 | 159 | 1.6 | 2.1 | 0 | 0 | 1 | 2 | 13 | |

*Data for the treatment groups DP3-84/30 and DP3-84/10 are from the first clinical study. Data from a third clinical study in which the corresponding non-estrogen-bridged extended cycle regimen (“Seasonale”) and a non-estrogen-bridged conventional 28-day regimen (“Nordette”) were administered are included for comparison.

TABLE 8
Number of Days of Unscheduled Bleeding and/or Spotting Per Cycle, By
Treatment Groups - Complete Cycles Only (Second Clinical Study)

| Regimen | | | | | | | | | | Median |
|---|---|---|---|---|---|---|---|---|---|---|
| (Treatment | | | | | | | | | | Per Monthly |
| Group)* | Cycle | N | Mean | SD | Min | Q1 | Median | Q3 | Max | Cycle |
| DP3-84/30 | 1 | 70 | 18.7 | 17.17 | 0 | 6 | 14.5 | 27 | 76 | 3.6 |
| | 2 | 54 | 10.4 | 8.36 | 0 | 4 | 8.5 | 16 | 38 | 2.1 |
| | 3 | 45 | 8.8 | 9.57 | 0 | 2 | 6 | 14 | 48 | 1.5 |
| | 4 | 36 | 9.2 | 8.54 | 0 | 2 | 6 | 14.5 | 28 | 1.5 |
| DP3-84/10 | 1 | 75 | 15.1 | 13.81 | 0 | 4 | 11 | 22 | 50 | 2.8 |
| | 2 | 59 | 9.3 | 10.02 | 0 | 2 | 6 | 16 | 45 | 1.5 |
| | 3 | 50 | 8.2 | 8.71 | 0 | 2 | 5 | 12 | 37 | 1.3 |
| | 4 | 41 | 10.0 | 11.15 | 0 | 2 | 6 | 16 | 50 | 1.5 |
| DP3-25/30 | 1 | 82 | 2.6 | 3.37 | 0 | 0 | 2 | 4 | 14 | 1.7 |
| | 2 | 78 | 6.4 | 6.06 | 0 | 1 | 5 | 10 | 25 | 4.2 |
| | 3 | 68 | 7.7 | 6.58 | 0 | 2.5 | 6 | 13 | 25 | 5.0 |
| | 4 | 66 | 5.8 | 5.57 | 0 | 2 | 5 | 8 | 24 | 4.2 |
| | 5 | 65 | 4.8 | 5.33 | 0 | 0 | 3 | 6 | 22 | 2.5 |
| | 6 | 59 | 4.9 | 5.14 | 0 | 1 | 4 | 7 | 25 | 3.4 |
| | 7 | 54 | 4.9 | 4.47 | 0 | 2 | 4 | 7 | 22 | 3.4 |
| | 8 | 51 | 4.8 | 4.96 | 0 | 1 | 4 | 7 | 20 | 3.4 |
| | 9 | 46 | 4.4 | 4.44 | 0 | 1 | 3 | 7 | 19 | 2.5 |
| | 10 | 42 | 5.7 | 5.36 | 0 | 1 | 4 | 8 | 20 | 3.4 |
| | 11 | 41 | 3.7 | 3.59 | 0 | 1 | 3 | 6 | 14 | 2.5 |
| | 12 | 39 | 4.1 | 5.22 | 0 | 0 | 3 | 6 | 20 | 2.5 |
| | 13 | 35 | 4.3 | 4.22 | 0 | 1 | 3 | 6 | 15 | 2.5 |
| Nordette | 1 | 85 | 2.5 | 3.46 | 0 | 0 | 1 | 3 | 18 | |
| | 2 | 80 | 1.9 | 2.27 | 0 | 0 | 1 | 3 | 13 | |
| | 3 | 72 | 1.8 | 2.11 | 0 | 0 | 1 | 3 | 8 | |
| | 4 | 69 | 1.4 | 1.65 | 0 | 0 | 1 | 2 | 8 | |
| | 5 | 67 | 1.4 | 1.93 | 0 | 0 | 1 | 2 | 10 | |
| | 6 | 61 | 1.5 | 1.97 | 0 | 0 | 1 | 2 | 8 | |
| | 7 | 59 | 1.6 | 2.42 | 0 | 0 | 1 | 2 | 11 | |
| | 8 | 57 | 1.6 | 2.70 | 0 | 0 | 1 | 2 | 14 | |
| | 9 | 53 | 1.9 | 2.80 | 0 | 0 | 1 | 2 | 13 | |
| | 10 | 52 | 1.9 | 2.47 | 0 | 0 | 1 | 3 | 9 | |
| | 11 | 49 | 1.2 | 1.43 | 0 | 0 | 1 | 2 | 6 | |
| | 12 | 47 | 1.0 | 1.93 | 0 | 0 | 0 | 1 | 10 | |
| | 13 | 45 | 2.6 | 2.98 | 0 | 1 | 2 | 4 | 14 | |

*Data for all treatment groups are from the second clinical study.

Tables 9 and 10 summarize the average number of days of withdrawal bleeding (defined as scheduled bleeding and/or spotting) per cycle by treatment group. Withdrawal bleeding includes any day for which the patient did not take a combination pill (days 85-91 for the DP3-84/30 and DP3-84/10 treatment groups, and days 26-28 for the DP3-25/30 treatment group). Table 11 summarizes the percentage of patients in the first and second clinical studies who experienced withdrawal bleeding during the withdrawal period.

TABLE 9
Number of Days of Scheduled Bleeding and/or Spotting Per
Cycle, By Treatment Groups - Complete Cycles Only

| Regimen | | | | | | | | | |
|---|---|---|---|---|---|---|---|---|---|
| (Treat- | | | | | | | | | |
| ment | | | | | | | | | |
| Group)* | Cycle | N | Mean | SD | Min | Q1 | Median | Q3 | Max |
| DP3-84/30 | 1 | 751 | 3.1 | 2.20 | 0 | 1 | 3 | 5 | 7 |
| | 2 | 609 | 2.8 | 2.15 | 0 | 1 | 3 | 4 | 7 |
| | 3 | 515 | 2.5 | 2.14 | 0 | 1 | 2 | 4 | 7 |
| | 4 | 420 | 2.5 | 2.22 | 0 | 0 | 2 | 4 | 7 |
| DP3-84/10 | 1 | 769 | 3.3 | 2.08 | 0 | 2 | 3 | 5 | 7 |
| | 2 | 625 | 3.1 | 2.05 | 0 | 1 | 3 | 5 | 7 |
| | 3 | 531 | 2.8 | 2.02 | 0 | 1 | 3 | 4 | 7 |
| | 4 | 443 | 2.7 | 2.03 | 0 | 1 | 3 | 4 | 7 |
| Seasonale | 1 | 385 | 4.0 | 2.1 | 0 | 3 | 4 | 6 | 7 |
| | 2 | 331 | 3.6 | 2.2 | 0 | 1 | 4 | 6 | 7 |
| | 3 | 296 | 3.4 | 2.2 | 0 | 2 | 4 | 5 | 7 |
| | 4 | 262 | 3.6 | 2.4 | 0 | 2 | 4 | 6 | 7 |
| Nordette | 1 | 214 | 3.4 | 1.5 | 0 | 3 | 3 | 4 | 7 |
| | 2 | 210 | 3.1 | 1.6 | 0 | 2 | 3 | 4 | 7 |
| | 3 | 204 | 3.1 | 1.6 | 0 | 2 | 3 | 4 | 7 |
| | 4 | 194 | 3.0 | 1.7 | 0 | 2 | 3 | 4 | 7 |
| | 5 | 188 | 3.0 | 1.7 | 0 | 2 | 3 | 4 | 7 |
| | 6 | 184 | 2.9 | 1.7 | 0 | 2 | 3 | 4 | 7 |
| | 7 | 178 | 2.8 | 1.5 | 0 | 2 | 3 | 4 | 6 |
| | 8 | 177 | 2.9 | 1.8 | 0 | 2 | 3 | 4 | 7 |
| | 9 | 172 | 2.9 | 1.6 | 0 | 2 | 3 | 4 | 6 |
| | 10 | 170 | 2.7 | 1.7 | 0 | 1 | 3 | 4 | 7 |
| | 11 | 163 | 2.7 | 1.7 | 0 | 2 | 3 | 4 | 6 |
| | 12 | 162 | 2.7 | 1.7 | 0 | 1 | 3 | 4 | 7 |
| | 13 | 159 | 3.3 | 1.9 | 0 | 2 | 3 | 5 | 7 |

*Data for the treatment groups DP3-84/30 and DP3-84/10 are from the first clinical study. Seasonale and Nordette data are from the third clinical study.

TABLE 10
Number of Days of Scheduled Bleeding and/or Spotting Per
Cycle, By Treatment Groups - Complete Cycles Only
(Second Clinical Study)

| Regimen | | | | | | | | | |
|---|---|---|---|---|---|---|---|---|---|
| (Treatment | | | | | | | | | |
| Group)* | Cycle | N | Mean | SD | Min | Q1 | Median | Q3 | Max |
| DP3-84/30 | 1 | 70 | 3.4 | 2.17 | 0 | 2 | 4 | 5 | 7 |
| | 2 | 54 | 2.4 | 2.03 | 0 | 0 | 2 | 4 | 6 |
| | 3 | 45 | 2.6 | 2.03 | 0 | 1 | 3 | 4 | 7 |
| | 4 | 36 | 2.8 | 2.07 | 0 | 1 | 2.5 | 4.5 | 7 |
| DP3-84/10 | 1 | 75 | 3.3 | 2.15 | 0 | 2 | 3 | 5 | 7 |
| | 2 | 59 | 2.7 | 2.39 | 0 | 0 | 3 | 5 | 7 |
| | 3 | 50 | 3.0 | 2.29 | 0 | 1 | 3 | 5 | 7 |
| | 4 | 41 | 3.0 | 2.47 | 0 | 1 | 2 | 5 | 7 |
| DP3-25/30 | 1 | 82 | 0.5 | 0.88 | 0 | 0 | 0 | 1 | 3 |
| | 2 | 78 | 0.7 | 1.13 | 0 | 0 | 0 | 1 | 3 |
| | 3 | 68 | 0.9 | 1.18 | 0 | 0 | 0 | 2 | 3 |
| | 4 | 66 | 0.6 | 1.05 | 0 | 0 | 0 | 1 | 3 |
| | 5 | 65 | 0.6 | 0.90 | 0 | 0 | 0 | 1 | 3 |
| | 6 | 59 | 0.8 | 1.09 | 0 | 0 | 0 | 2 | 3 |
| | 7 | 54 | 0.7 | 1.04 | 0 | 0 | 0 | 1 | 3 |
| | 8 | 51 | 0.6 | 0.87 | 0 | 0 | 0 | 1 | 3 |
| | 9 | 46 | 0.4 | 0.68 | 0 | 0 | 0 | 1 | 3 |
| | 10 | 42 | 0.7 | 1.12 | 0 | 0 | 0 | 1 | 3 |
| | 11 | 41 | 0.4 | 0.83 | 0 | 0 | 0 | 0 | 3 |
| | 12 | 39 | 0.4 | 0.74 | 0 | 0 | 0 | 1 | 3 |
| | 13 | 35 | 0.7 | 0.96 | 0 | 0 | 0 | 1 | 3 |
| Nordette | 1 | 85 | 3.2 | 1.76 | 0 | 2 | 3 | 4 | 7 |
| | 2 | 80 | 3.0 | 1.72 | 0 | 2 | 3 | 4 | 7 |
| | 3 | 72 | 2.9 | 1.68 | 0 | 2 | 3 | 4 | 7 |
| | 4 | 69 | 2.7 | 1.72 | 0 | 1 | 3 | 4 | 6 |
| | 5 | 67 | 2.5 | 1.63 | 0 | 1 | 2 | 4 | 5 |
| | 6 | 61 | 2.7 | 1.78 | 0 | 1 | 3 | 4 | 7 |
| | 7 | 59 | 2.7 | 2.01 | 0 | 1 | 3 | 4 | 7 |
| | 8 | 57 | 2.6 | 2.00 | 0 | 1 | 3 | 4 | 7 |
| | 9 | 53 | 2.8 | 1.69 | 0 | 2 | 3 | 4 | 6 |
| | 10 | 52 | 2.6 | 1.83 | 0 | 1 | 3 | 4 | 7 |
| | 11 | 49 | 2.7 | 1.73 | 0 | 2 | 3 | 4 | 7 |
| | 12 | 47 | 2.7 | 1.66 | 0 | 1 | 3 | 4 | 6 |
| | 13 | 45 | 2.9 | 2.01 | 0 | 1 | 3 | 4 | 7 |

*Data for all treatment groups are from the first clinical study.

TABLE 11
Percentage of Patients Reporting Bleeding and/or Spotting
During the Scheduled Withdrawal Bleeding Period.

| | Regimen | | Number of | | |
|---|---|---|---|---|---|
| Clinical | (Treatment | | Complete | | |
| Study | Group) | Cycle | Cycles | N | (%) |
| First | DP3-84/30 | 1 | 751 | 630 | (83.9) |
| | | 2 | 609 | 481 | (79.0) |
| | | 3 | 515 | 390 | (75.7) |
| | | 4 | 420 | 304 | (72.4) |
| | DP3-84/10 | 1 | 769 | 659 | (85.7) |
| | | 2 | 625 | 530 | (84.8) |
| | | 3 | 531 | 430 | (81.0) |
| | | 4 | 443 | 361 | (81.5) |
| Second | DP3-84/30 | 1 | 70 | 59 | (84.3) |
| | | 2 | 54 | 40 | (74.1) |
| | | 3 | 45 | 35 | (77.8) |
| | | 4 | 36 | 32 | (88.9) |
| | DP3-84/10 | 1 | 75 | 64 | (85.3) |
| | | 2 | 59 | 41 | (69.5) |
| | | 3 | 50 | 40 | (80.0) |
| | | 4 | 41 | 32 | (78.0) |
| | DP3-25/30 | 1 | 82 | 21 | (25.6) |
| | | 2 | 78 | 25 | (32.1) |
| | | 3 | 68 | 27 | (39.7) |
| | | 4 | 66 | 22 | (33.3) |
| | | 5 | 65 | 22 | (33.8) |
| | | 6 | 59 | 23 | (39.0) |
| | | 7 | 54 | 20 | (37.0) |
| | | 8 | 51 | 20 | (39.2) |
| | | 9 | 46 | 14 | (30.4) |
| | | 10 | 42 | 14 | (33.3) |
| | | 11 | 41 | 9 | (22.0) |
| | | 12 | 39 | 10 | (25.6) |
| | | 13 | 35 | 16 | (45.7) |
| | Nordette | 1 | 85 | 75 | (88.2) |
| | | 2 | 80 | 72 | (90.0) |
| | | 3 | 72 | 65 | (90.3) |
| | | 4 | 69 | 60 | (87.0) |
| | | 5 | 67 | 56 | (83.6) |
| | | 6 | 61 | 53 | (86.9) |
| | | 7 | 59 | 47 | (79.7) |
| | | 8 | 57 | 44 | (77.2) |
| | | 9 | 53 | 46 | (86.8) |
| | | 10 | 52 | 43 | (82.7) |
| | | 11 | 49 | 41 | (83.7) |
| | | 12 | 47 | 41 | (87.2) |
| | | 13 | 45 | 38 | (84.4) |

Table 12 presents the percentage of patients in each treatment group who reported no bleeding and/or spotting during the defined withdrawal bleeding period. Data from both the first and second clinical studies are presented. Table 13 presents the percentage of patients in each treatment group from the first clinical study who did not report any bleeding and/or spotting during the entire cycle. Table 13 also presents the percentage of patients in the Seasonale and Nordette treatment groups in the third clinical study who reported no bleeding and/or spotting during the entire cycle.

TABLE 12
Percentage of Patients Not Reporting Bleeding and/or Spotting
During the Scheduled Withdrawal Bleeding Period.

| | | | Number of | | |
|---|---|---|---|---|---|
| Clinical | Regimen | | Complete | | |
| Study | (Treatment Group) | Cycle | Cycles | N | (%) |
| First | DP3-84/30 | 1 | 751 | 121 | (16.1) |
| | | 2 | 609 | 128 | (21.0) |
| | | 3 | 515 | 125 | (24.3) |
| | | 4 | 420 | 116 | (27.6) |
| | DP3-84/10 | 1 | 769 | 110 | (14.3) |
| | | 2 | 625 | 95 | (15.2) |
| | | 3 | 531 | 101 | (19.0) |
| | | 4 | 443 | 82 | (18.5) |
| Second | DP3-84/30 | 1 | 70 | 11 | (15.7) |
| | | 2 | 54 | 14 | (25.9) |
| | | 3 | 45 | 10 | (22.2) |
| | | 4 | 36 | 4 | (11.1) |
| | DP3-84/10 | 1 | 75 | 11 | (14.7) |
| | | 2 | 59 | 18 | (30.5) |
| | | 3 | 50 | 10 | (20.0) |
| | | 4 | 41 | 9 | (22.0) |
| | DP3-25/30 | 1 | 82 | 61 | (74.4) |
| | | 2 | 78 | 53 | (67.9) |
| | | 3 | 68 | 41 | (60.3) |
| | | 4 | 66 | 44 | (66.7) |
| | | 5 | 65 | 43 | (66.2) |
| | | 6 | 59 | 36 | (61.0) |
| | | 7 | 54 | 34 | (63.0) |
| | | 8 | 51 | 31 | (60.8) |
| | | 9 | 46 | 32 | (69.6) |
| | | 10 | 42 | 28 | (66.7) |
| | | 11 | 41 | 32 | (78.0) |
| | | 12 | 39 | 29 | (74.4) |
| | | 13 | 35 | 19 | (54.3) |
| | Nordette | 1 | 85 | 10 | (11.8) |
| | | 2 | 80 | 8 | (10.0) |
| | | 3 | 72 | 7 | (9.7) |
| | | 4 | 69 | 9 | (13.0) |
| | | 5 | 67 | 11 | (16.4) |
| | | 6 | 61 | 8 | (13.1) |
| | | 7 | 59 | 12 | (20.3) |
| | | 8 | 57 | 13 | (22.8) |
| | | 9 | 53 | 7 | (13.2) |
| | | 10 | 52 | 9 | (17.3) |
| | | 11 | 49 | 8 | (16.3) |
| | | 12 | 47 | 6 | (12.8) |
| | | 13 | 45 | 7 | (15.6) |

TABLE 13
Percentage of Patients Not Reporting Bleeding and/or
Spotting During a Cycle.

| | Regimen | | Number of | | |
|---|---|---|---|---|---|
| Clinical | (Treatment | | Complete | | |
| Study | Group) | Cycle | Cycles | N | (%) |
| First | DP3-84/30 | 1 | 751 | 24 | (3.2) |
| | | 2 | 609 | 23 | (3.8) |
| | | 3 | 515 | 23 | (4.5) |
| | | 4 | 420 | 33 | (7.9) |
| | DP3-84/10 | 1 | 769 | 16 | (2.1) |
| | | 2 | 625 | 28 | (4.5) |
| | | 3 | 531 | 33 | (6.2) |
| | | 4 | 443 | 28 | (6.3) |
| Third | Seasonale | 1 | 385 | 4 | (1.0) |
| | | 2 | 331 | 6 | (1.8) |
| | | 3 | 296 | 11 | (3.7) |
| | | 4 | 262 | 9 | (3.4) |
| | Nordette | 1 | 214 | 6 | (2.8) |
| | | 2 | 210 | 5 | (2.3) |
| | | 3 | 204 | 13 | (6.4) |
| | | 4 | 194 | 11 | (5.7) |
| | | 5 | 188 | 8 | (543) |
| | | 6 | 184 | 7 | (3.8) |
| | | 7 | 178 | 10 | (5.6) |
| | | 8 | 177 | 16 | (9.0) |
| | | 9 | 172 | 12 | (7.0) |
| | | 10 | 170 | 12 | (7.1) |
| | | 11 | 163 | 10 | (6.1) |
| | | 12 | 162 | 9 | (5.6) |
| | | 13 | 159 | 11 | (6.9) |

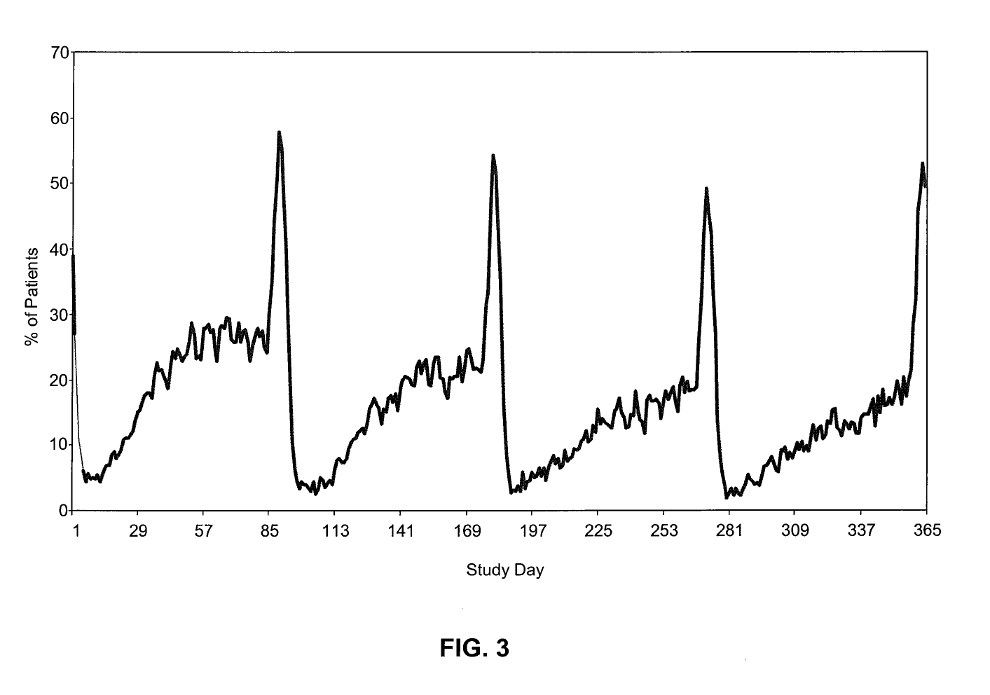

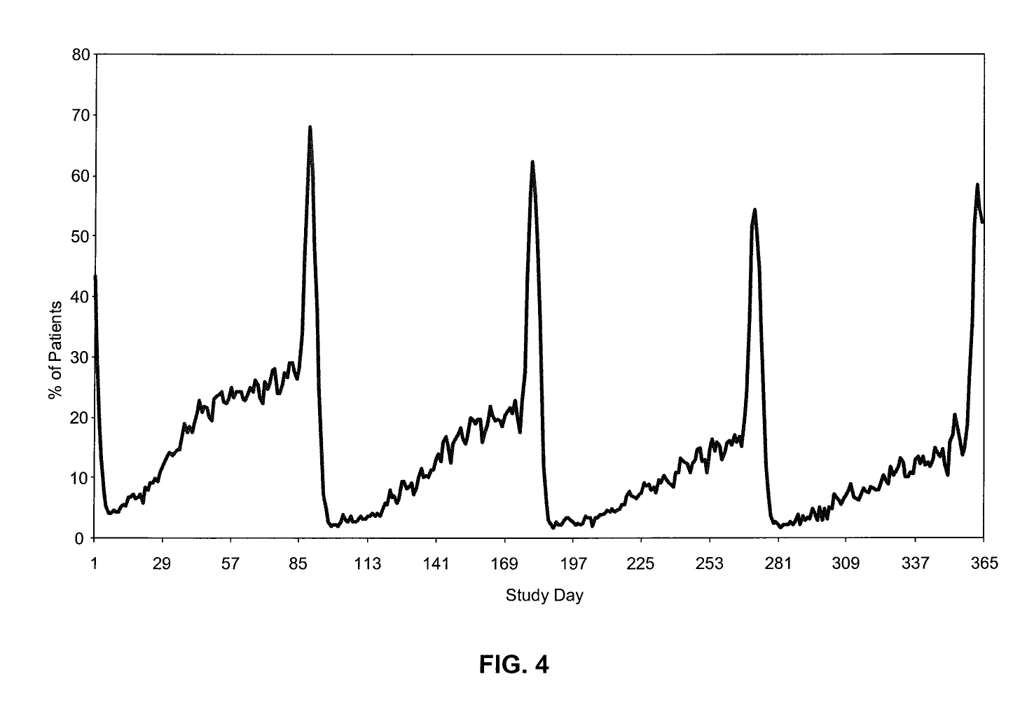

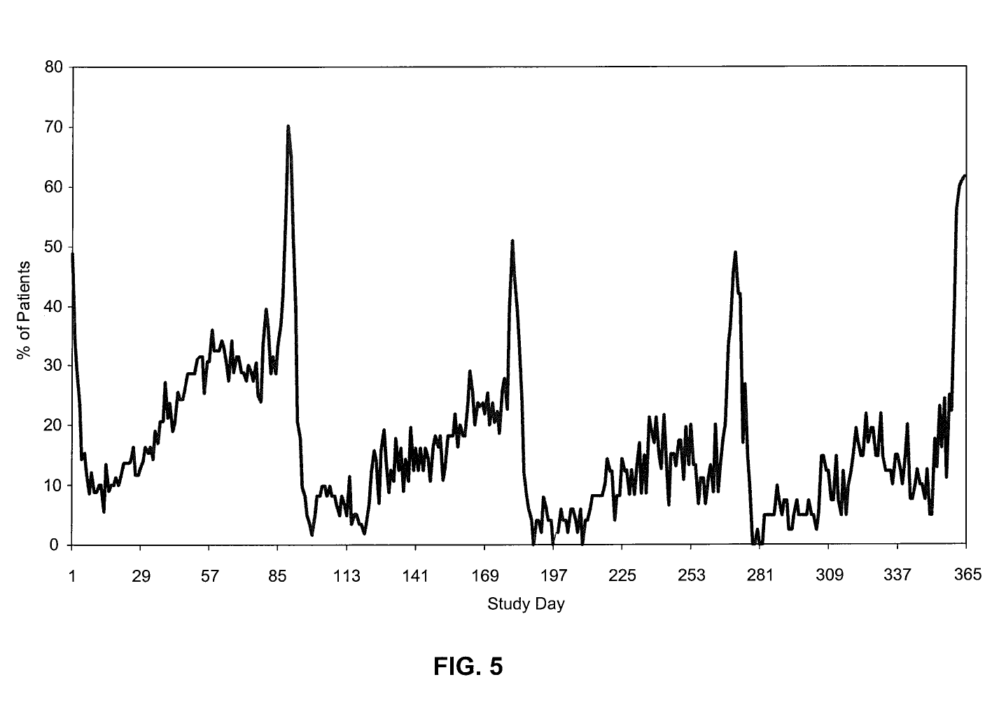

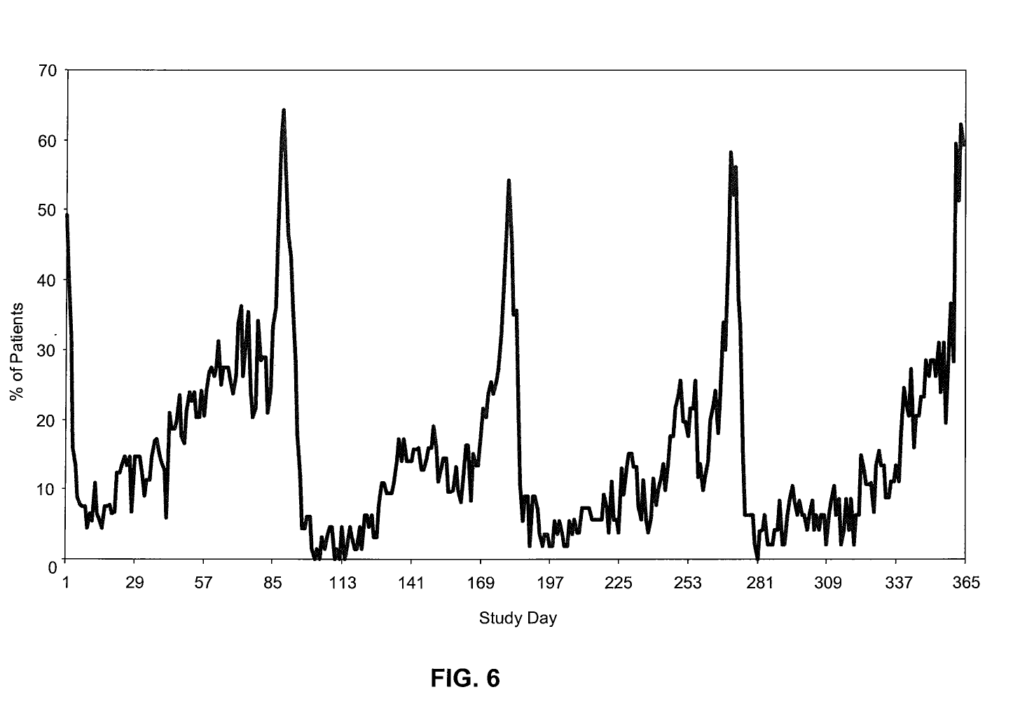

FIGS. 3 through 6 show the distribution of bleeding and spotting among patients in the various treatment groups from the first and second clinical studies. FIGS. 3 and 4 present data for patients from the DP3-84/30 and DP3-84/10 treatment groups, respectively, from the first clinical study. FIGS. 4, 5, and 6 present data for patients from the DP3-84/30, DP3-84/10, and DP3-25/30 treatment groups from the second clinical study.

### Example 8

Adverse events reported by patients during the course of the clinical studies of Example 7 were recorded. An “adverse event” was defined as any reaction, side effect, or other undesirable event that occurred in conjunction with the use of the drug, biological product or diagnostic agent during the study, whether or not the event was considered to be related to the study drug (see the protocol in Example 5). The percentage of patients in the first and second clinical studies reporting certain adverse events are presented in Tables 14 through 20. Each table also includes similar data from the third clinical study.

Tables 14 and 15 present the percentage of patients in the first and second clinical studies who reported menorrhagia and dysmenorrhoea as adverse events. Corresponding data from the third clinical study are also presented. The adverse event coding (MedDRA) may have varied between the first and third clinical studies, so that some of the adverse event reports in the first clinical study that could have been identified as menorrhagia may have been identified as intermenstrual bleeding.

TABLE 14
Incidence of Menorrhagia, by Treatment Group and Study

| | Regimen | | |
|---|---|---|---|
| | (Treatment | | |
| Clinical Study | Group) | N | (%) |
| First | DP3-84/30 | 80 | (8.90) |
| | DP3-84/10 | 61 | (6.79) |
| Third | Seasonale | 53 | (11.6) |
| | Nordette | 6 | (2.7) |
| Second | DP3-84/30 | 7 | (7.6) |
| | DP3-84/10 | 4 | (4.3) |
| | DP3-25/30 | 2 | (2.3) |
| | Nordette | 2 | (2.3) |

TABLE 15
Incidence of Dysmenorrhoea, by Treatment Group and Study

| | Regimen | | |
|---|---|---|---|
| | (Treatment | | |
| Clinical Study | Group) | N | (%) |
| First | DP3-84/30 | 40 | (4.5) |
| | DP3-84/10 | 37 | (4.1) |
| Third | Seasonale | 26 | (5.7) |
| | Nordette | 9 | (4.0) |
| Second | DP3-84/30 | 3 | (3.3) |
| | DP3-84/10 | 2 | (2.1) |
| | DP3-25/30 | 1 | (1.1) |
| | Nordette | 4 | (4.5) |

Table 16 presents the percentage of patients in the first and second clinical studies who reported acne as an adverse event. The data include all reports of “Acne NOS,” “Acne Aggravated,” or “Acne Cystic” as adverse events. (“NOS” refers to “no other symptom.”) Corresponding data from the third clinical study are also included.

TABLE 16
Incidence of Acne, by Treatment Group and Study

| | Regimen | | |
|---|---|---|---|
| | (Treatment | | |
| Clinical Study | Group) | N | (%) |
| First | DP3-84/30 | 57 | (6.3) |
| | DP3-84/10 | 54 | (6.0) |
| Third | Seasonale | 21 | (4.6) |
| | Nordette | 10 | (4.4) |
| Second | DP3-84/30 | 3 | (3.3) |
| | DP3-84/10 | 9 | (9.6) |
| | DP3-25/30 | 2 | (2.3) |
| | Nordette | 2 | (2.3) |

Table 17 presents the percentage of patients in the first and second clinical studies who reported the following categories of infections as adverse events:
“Bladder Infection NOS”
“Nasopharyngitis” (including “Pharyngitis Streptococcal”)
“Sinusitis NOS” (including “Sinusitis Acute NOS”)
URI (including “Upper Respiratory Tract Infection NOS”; “Upper Respiratory Tract Infection Viral NOS”; and “Respiratory Tract Infection NOS”)
UTI (including “Urinary Tract Infection NOS”; and “Urinary Tract Infection bacterial NOS)”
“Vaginitis NOS” (including “Vaginitis Bacterial NOS; and Vulvovaginitis NOS”)
“Vaginosis Fungal NOS”
   Corresponding data from the third clinical study are also included.

TABLE 17
Incidence of Infection, by Treatment Group and Study

| | | Regimen | | |
|---|---|---|---|---|
| | | (Treatment | | |
| Infection | Study | Group) | N | (%) |
| Bladder | First | DP3-84/30 | 10 | (1.1) |
| Infection | | DP3-84/10 | 10 | (1.1) |
| | Third | Seasonale | 6 | (1.3) |
| | | Nordette | 7 | (3.1) |
| | Second | DP3-84/30 | 0 | |
| | | DP3-84/10 | 0 | |
| | | DP3-25/30 | 0 | |
| | | Nordette | 1 | (1.1) |
| Nasopharyngitis | First | DP3-84/30 | 98 | (10.9) |
| | | DP3-84/10 | 103 | (11.5) |
| | Third | Seasonale | 106 | (23.2) |
| | | Nordette | 70 | (31.0) |
| | Second | DP3-84/30 | 17 | (18.5) |
| | | DP3-84/10 | 13 | (13.8) |
| | | DP3-25/30 | 14 | (16.1) |
| | | Nordette | 13 | (14.8) |
| Sinusitis | First | DP3-84/30 | 66 | (7.3) |
| | | DP3-84/10 | 65 | (7.2) |
| | Third | Seasonale | 46 | (10.1) |
| | | Nordette | 25 | (11.1) |
| | Second | DP3-84/30 | 1 | (1.1) |
| | | DP3-84/10 | 7 | (7.4) |
| | | DP3-25/30 | 4 | (4.6) |
| | | Nordette | 3 | (3.4) |
| URI | First | DP3-84/30 | 43 | (4.8) |
| | | DP3-84/10 | 54 | (6.0) |
| | Third | Seasonale | 29 | (6.4) |
| | | Nordette | 24 | (10.6) |
| | Second | DP3-84/30 | 1 | (1.1) |
| | | DP3-84/10 | 5 | (5.3) |
| | | DP3-25/30 | 3 | (3.4) |
| | | Nordette | 1 | (1.1) |
| UTI | First | DP3-84/30 | 36 | (4.0) |
| | | DP3-84/10 | 45 | (5.0) |
| | Third | Seasonale | 20 | (4.4) |
| | | Nordette | 14 | (6.2) |
| | Second | DP3-84/30 | 2 | (2.2) |
| | | DP3-84/10 | 5 | (5.3) |
| | | DP3-25/30 | 6 | (6.9) |
| | | Nordette | 7 | (8.0) |
| Vaginitis | First | DP3-84/30 | 16 | (1.8) |
| | | DP3-84/10 | 22 | (2.5) |
| | Third | Seasonale | 11 | (2.4) |
| | | Nordette | 5 | (2.2) |
| | Second | DP3-84/30 | 2 | (2.2) |
| | | DP3-84/10 | 5 | (5.3) |
| | | DP3-25/30 | 3 | (3.4) |
| | | Nordette | 4 | (4.5) |
| Vaginosis | First | DP3-84/30 | 25 | (2.8) |
| | | DP3-84/10 | 20 | (2.2) |
| | Third | Seasonale | 9 | (2.0) |
| | | Nordette | 4 | (1.8) |
| | Second | DP3-84/30 | 2 | (2.2) |
| | | DP3-84/10 | 1 | (1.1) |
| | | DP3-25/30 | 3 | (3.4) |
| | | Nordette | 1 | (1.1) |

Table 18 presents the percentage of patients in the first and second clinical studies who reported headache as an adverse event. The data include all reports of “Headache NOS,” “Tension Headache,” “Sinus Headache,” and “Headache NOS Aggravated” as adverse events. Data from the third clinical study is also included.

TABLE 18
Incidence of Headache, by Treatment Group and Study

| | Regimen | | |
|---|---|---|---|
| | (Treatment | | |
| Study | Group) | N | (%) |
| First | DP3-84/30 | 63 | (7.0) |
| | DP3-84/10 | 58 | (6.5) |
| Third | Seasonale | 110 | (24.1) |
| | Nordette | 79 | (35.0) |
| Second | DP3-84/30 | 8 | (8.7) |
| | DP3-84/10 | 4 | (4.3) |
| | DP3-25/30 | 7 | (8.0) |
| | Nordette | 5 | (5.7) |

Table 19 presents the percentage of patients in the first and second clinical studies who reported nausea as an adverse event. The data include all reports of “Nausea” and Nausea Aggravated” as adverse events. Data from the third clinical study is also included.

TABLE 19
Incidence of Nausea, by Treatment Group and Study

| | Regimen | | |
|---|---|---|---|
| | (Treatment | | |
| Study | Group) | N | (%) |
| First | DP3-84/30 | 51 | (5.7) |
| | DP3-84/10 | 47 | (5.2) |
| Third | Seasonale | 34 | (7.5) |
| | Nordette | 21 | (9.3) |
| Second | DP3-84/30 | 5 | (5.4) |
| | DP3-84/10 | 3 | (3.2) |
| | DP3-25/30 | 2 | (2.3) |
| | Nordette | 6 | (6.8) |

Table 20 presents the percentage of patients in the first and second clinical studies who reported depression as an adverse event. The data include all reports of “Depression” and “Depression Aggravated” as adverse events. Data from the third clinical study is also included.

TABLE 20
Incidence of Depression, by Treatment Group and Study

| | Regimen | | |
|---|---|---|---|
| | (Treatment | | |
| Study | Group) | N | (%) |
| First | DP3-84/30 | 35 | (3.9) |
| | DP3-84/10 | 38 | (4.2) |
| Third | Seasonale | 10 | (2.2) |
| | Nordette | 14 | (6.2) |
| Second | DP3-84/30 | 4 | (4.3) |
| | DP3-84/10 | 4 | (4.3) |
| | DP3-25/30 | 2 | (2.3) |
| | Nordette | 1 | (1.1) |

### Example 9

Preliminary calculations of the incidence of pregnancy were performed for patients participating in the first clinical study of Examples 7 and 8. The results are presented in Tables 21, 22 and 23.

Table 21 presents the number of patients weighing 70 kg or more, and those weighing less than 70 kg, who became pregnant during the course of administration of the DP3-84/30 or DP3-84/10 regimen in the first clinical study.

TABLE 21
Pearl Index Calculations of Treatment Failure Rates:
For all Patients, by Weight and Treatment - Completed
Cycles Only (First Clinical Study)

| | | | | Number of | | |
|---|---|---|---|---|---|---|
| Regimen | | | Number of | 28-Day | Number of | |
| (Treatment | Weight | | Complete | Patient | On-Drug | Pearl |
| Group) | Category | | Cycles | Months | Pregnancies | Index |
| DP3-84/30 | <70 | kg | 1372 | 4459.0 | 13 | 3.79 |
| | ≧70 | kg | 918 | 2983.5 | 6 | 2.61 |
| DP3-84/10 | <70 | kg | 1380 | 4485.0 | 5 | 1.45 |
| | ≧70 | kg | 986 | 3204.5 | 3 | 1.22 |

Patients in the first clinical study were also monitored after the end of the study for the occurrence of pregnancy. Tables 22 and 23 present the number of pregnancies that occurred for patients in the DP3-84/30 and DP3-84/10 treatment groups after the last dose administered at the end of the study, within two weeks of the last dose, and within one month of the last dose. The values in Tables 22 and 23 are preliminary pending confirmation of the last date of study medication and date of conception. The data is presented based on the last dose taken from the Pregnancy Information obtained during the study, or from the Case Report Form Pregnancy Information.

TABLE 22
Preliminary Number of Pregnancies Based on Last Dose from Pregnancy
Information

| Regimen | | | Within 2 | Within 1 |
|---|---|---|---|---|
| (Treatment | Total Number | After Last | Weeks of | Month of Last |
| Group) | of Pregnancies | Dose | Last Dose | Dose |
| DP3-84/30 | 36 | 19 (52.8%) | 5 (13.9%) | 11 (30.6%) |
| DP3-84/10 | 22 | 15 (68.2%) | 4 (18.2%) | 10 (45.5%) |

TABLE 23
Preliminary Number of Pregnancies Based on Last Dose from Case
Report Form Pregnancy Information

| Regimen | | | Within 2 | Within 1 |
|---|---|---|---|---|
| (Treatment | Total Number | After Last | Weeks of | Month of Last |
| Group) | of Pregnancies | Dose | Last Dose | Dose |
| DP3-84/30 | 36 | 19 (52.8%) | 4 (11.1%) | 11 (30.6%) |
| DP3-84/10 | 22 | 14 (63.6%) | 3 (13.6%) | 9 (40.9%) |

### Example 10

A single-dose, open-label, one period pharmacokinetic study was conducted with 24 healthy, female, adult subjects to evaluate the single dose and steady-state pharmacokinetics of tablets containing 0.150 mg levonorgestrel and 0.030 mg ethinyl estradiol. Study participants were required to take one tablet (1×0.150 mg levonorestrel/0.030 mg ethinyl estradiol) once a day for 84 consecutive days followed by 7 days of one tablet of containing only 0.030 mg of ethinyl estradiol.

Following dosing on Day 1, serial blood samples were collected pre-dose and at intervals over 24 hours after dosing. On Study Day 18, 19 and 20, samples were collected prior to dosing. On Day 21, samples were collected pre-dose and at intervals over 24 hours post-dose. On Study Day 81, 82, and 83, samples were collected prior to dosing. On Day 84, samples were collected pre-dose and at intervals thereafter. The plasma concentrations of levonorgestrel and ethinyl estradiol were measured using fully validated analytical procedures. On Study Days 88, 89, 90, and 91, samples were collected prior to dosing with the ethinyl estradiol only tablets. On Study Day 91, samples were collected prior to dosing and for 96 hours post-dosing. Samples from the pre-dose sample on Day 91 and later were analyzed for plasma concentration of ethinyl estradiol only.

During the course of the study, blood samples were also collected and analyzed for Follicle Stimulating Hormone (FSH), estradiol, Luteinizing Hormone (LH), free testosterone, and total testosterone predose on Days 1, 21, 84, 91, and on Days 98, 105, 119, 133, and 147. Plasma concentrations of each hormone were measured using standard commercially available clinical assays.

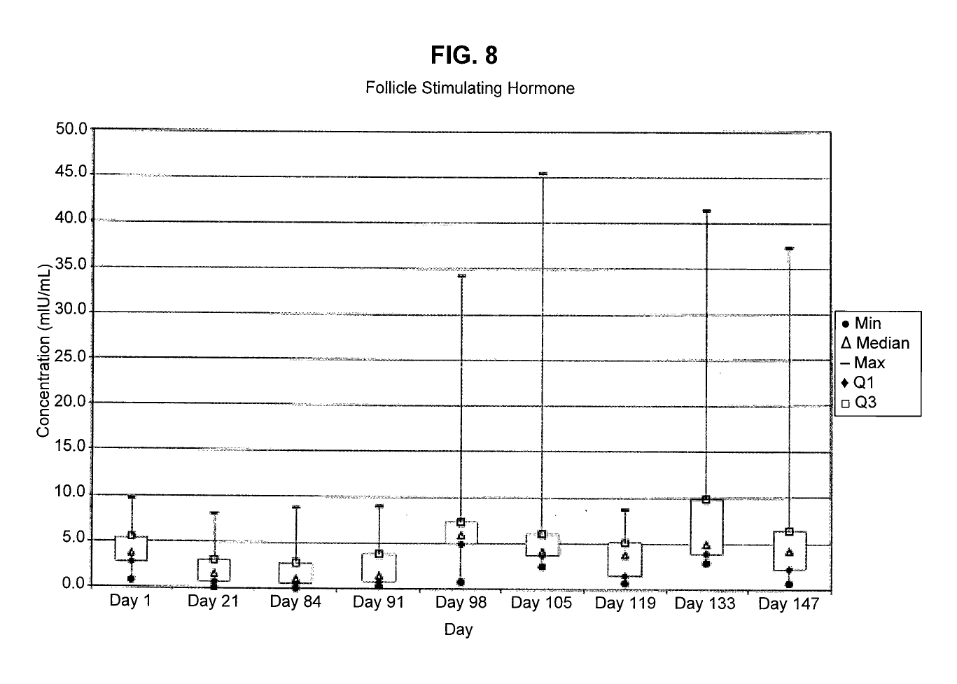

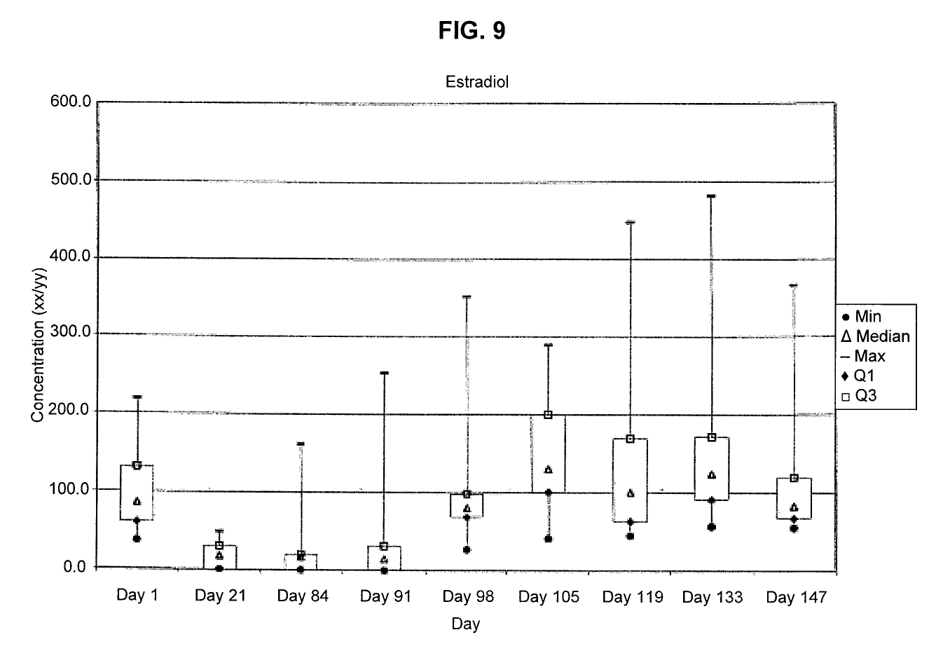

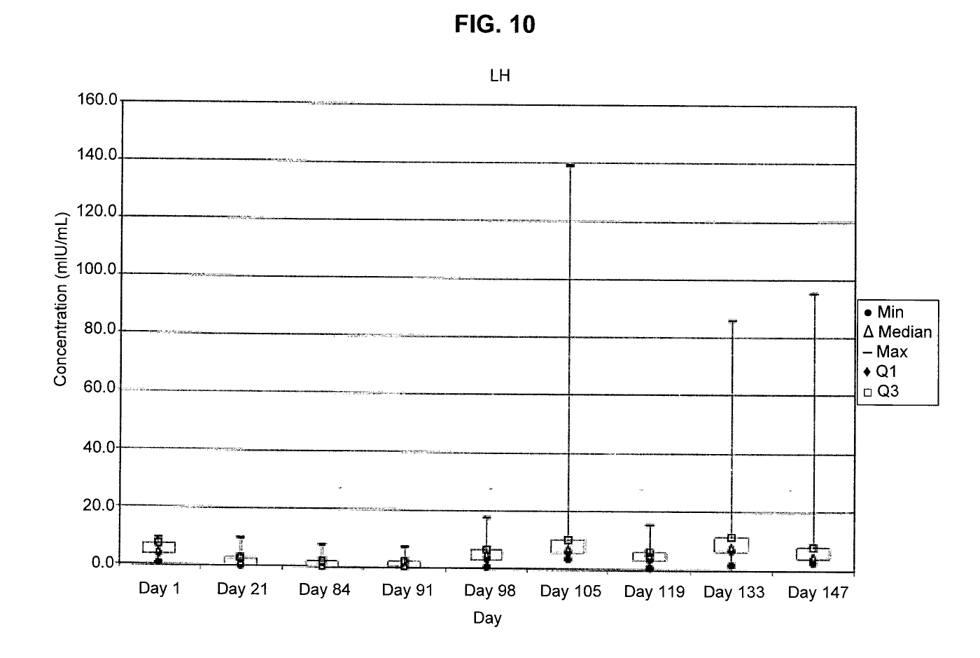

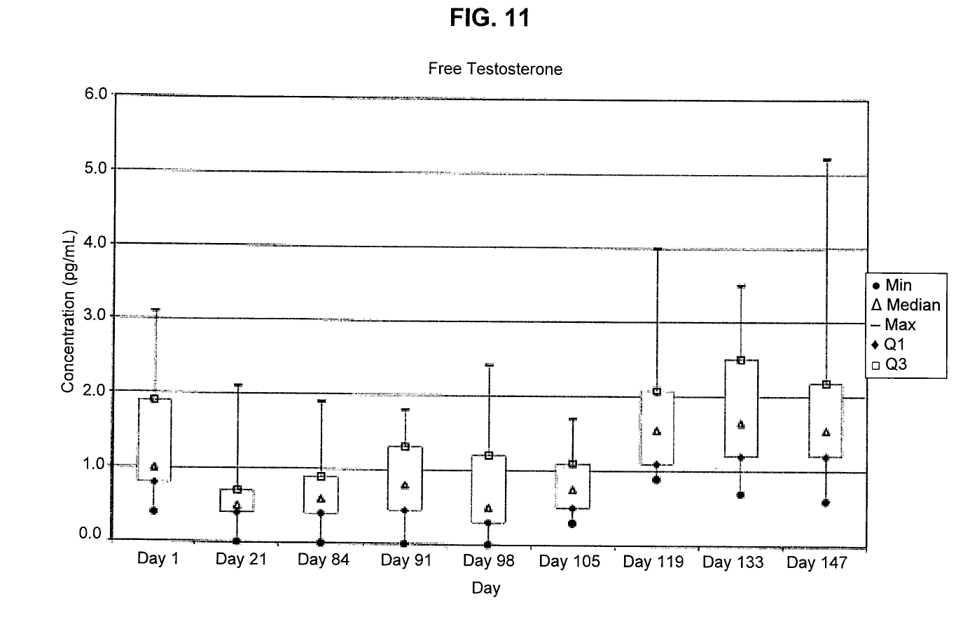

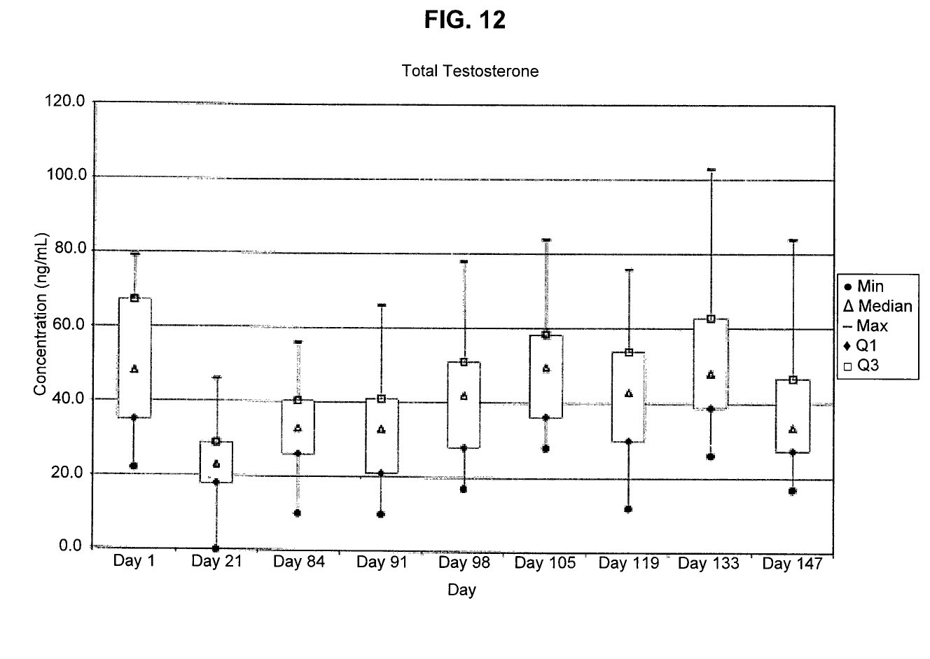

FIGS. 8 through 12 present the minimum (“Min”), maximum (“Max”), and median plasma concentrations of FSH, estradiol, LH, free testosterone and total testosterone of the patients during the course of this study, during 84 days of administration of the levonorgestrel/ethinyl estradiol, followed by 7 days of administration of ethinyl estradiol. Plasma concentrations of the hormones are also shown up to about 56 days (to Day 147) after completion of administration, during which no hormones were administered to the patients. The plasma concentration of each hormone at Day 1 represents the plasma concentration measured before the beginning of administration of the levonorgestrel/ethinyl estradiol regimen, and thus represents baseline plasma concentrations.

### Example 11

Another aspect of the invention encompasses the reduction of unscheduled bleeding and/or spotting by administration of a bridged cycle regimen as a run-in regimen. Groups of women will be administered one of the following run-in bridged regimens: (1) a regimen comprising (a) 84 oral contraceptive tablets on days 1 through 84 of the menstrual cycle, one tablet per day, each tablet containing 150 μg levonorgestrel and 30 μg ethinyl estradiol, and (b) 7 tablets on days 85 though 91 of the cycle, one tablet per day, each tablet containing 30 μg ethinyl estradiol; (2) a regimen comprising (a) 84 oral contraceptive tablets on days 1 through 84 of the menstrual cycle, one tablet per day, each tablet containing 150 μg levonorgestrel and 30 μg ethinyl estradiol, and (b) 7 tablets on days 85 though 91 of the cycle, one tablet per day, each tablet containing 10 μg ethinyl estradiol; or (3) a regimen comprising (a) 25 oral contraceptive tablets on days 1 through 25 of the menstrual cycle, one tablet per day, each tablet containing 150 μg levonorgestrel and 30 μg ethinyl estradiol, and (b) 3 tablets on days 26 though 28 of the cycle, one tablet per day, each tablet containing 30 μg ethinyl estradiol.

Following administration of the run-in regimens for 2 cycles, each group will then receive one of the following lower dose hormone regimens: (1) an extended bridged cycle regimen in which a combination of 100 μg levonorgestrel and 20 μg ethinyl estradiol is administered for 84 days followed by 7 days of 10 μg ethinyl estradiol; (2) an extended cycle regimen in which a combination of 100 μg levonorgestrel and 20 μg ethinyl estradiol is administered for 84 days followed by 7 days of placebo; (3) a 28-day regimen containing a reduced amount of estrogen and/or progestin; (4) an extended regimen other than as disclosed herein containing a reduced amount of estrogen and/or progestin; or (5) a bridged regimen in which a combination of 150 μg desogestrel and 20 μg ethinyl estradiol is administered for 21 days followed by 7 days of 10 μg ethinyl estradiol.

The lower dose hormone regimens will be administered continuously and consecutively for one or more cycles following the run-in period. Groups of women will be administered the lower dose hormone regimen for 3, 4, 5, or 6 cycles following the run-in period.

Each group of women in which a lower dose hormone regimen is administered after the run-in period will experience a reduction in the median number of days of unscheduled bleeding and/or spotting at earlier cycles as compared to the median number of days of unscheduled bleeding and/or spotting observed at corresponding cycles in the absence of the run-in period.

Application of the compounds, compositions and methods of the present invention for the medical or pharmaceutical uses described can be accomplished by any clinical, medical, and pharmaceutical methods and techniques as are presently or prospectively known to those skilled in the art. It will therefore be appreciated that the various embodiments which have been described above are intended to illustrate the invention and various changes and modifications can be made in the inventive method without departing from the spirit and scope thereof.

All documents, e.g., scientific publications, patents, patent applications and patent publications recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A method of reducing unscheduled bleeding or spotting during administration of a hormone regimen in a female, the method comprising:
(a) administering to the female one or more cycles of a run-in regimen,
wherein each cycle of the regimen comprises administering to the female a combination of estrogen and progestin for 60 to 110 consecutive days,
wherein the daily amount of estrogen administered for 60 to 110 consecutive days is equivalent to about 5 μg to about 50 μg of ethinyl estradiol,
wherein the daily amount of progestin administered for 60 to 110 consecutive days is equivalent to about 0.05 mg to about 1.5 mg of levonorgestrel,
wherein the one or more cycles of (a) is followed by
(b) administering to the female one or more cycles of a lower dose hormone regimen,
wherein the daily amount of hormone administered during any period of a cycle comprising administration of hormone is selected from the group consisting of: a daily amount of estrogen that is lower than the daily amount of estrogen administered for 60 to 110 consecutive days in (a); a daily amount of progestin that is lower than the daily amount of progestin administered for 60 to 110 consecutive days in (a); and combinations thereof,
wherein the daily amounts are contraceptive or therapeutic amounts, and
wherein unscheduled bleeding or spotting during administration of the lower dose hormone regimen of (b) is reduced following prior administration of the run-in regimen of (a).

2. The method of claim 1, wherein the combination of estrogen and progestin in (a) is administered for 81 to 110 consecutive days.

3. The method of claim 2, wherein the combination of estrogen and progestin in (a) is administered for 81 to 89 consecutive days.

4. The method of claim 3, wherein the combination of estrogen and progestin in (a) is administered for 84 consecutive days.

5. The method of claim 1, wherein the daily amount of estrogen in (a) is equivalent to about 10 μg to about 30 μg of ethinyl estradiol, and the daily amount of progestin in (a) is equivalent to about 50 μg to about 150 μg of levonorgestrel.

6. The method of claim 5, wherein the daily amount of estrogen in (a) is equivalent to about 30 μg of ethinyl estradiol, and the daily amount of progestin in (a) is equivalent to about 150 μg of levonorgestrel.

7. The method of claim 1, wherein the estrogen in (a), (b), or (a) and (b) is ethinyl estradiol.

8. The method of claim 1, wherein the progestin in (a), (b), or (a) and (b) is levonorgestrel.

9. The method of claim 1, wherein the progestin in (a), (b), or (a) and (b) is desogestrel.

10. The method of claim 1, wherein each cycle of the run-in regimen of (a) comprises:
(i) administering the combination of estrogen and progestin for 60 to 110 consecutive days, followed by
(ii) a period of 2 to 10 consecutive days selected from the group consisting of: a hormone-free period of 2 to 10 consecutive days, wherein neither estrogen nor progestin is administered to the female during the hormone-free period; and a period of 2 to 10 consecutive days wherein estrogen is administered without progestin.

11. The method of claim 10, wherein the period in (a)(ii) is a period of 7 consecutive days.

12. The method of claim 10, wherein the period in (a)(ii) is a hormone-free period.

13. The method of claim 12, wherein the hormone-free period is achieved by administering a hormone-free placebo.

14. The method of claim 10, wherein the period in (a)(ii) is a period of 2 to 10 consecutive days wherein estrogen is administered without progestin.

15. The method of claim 14, wherein the daily amount of estrogen in (a)(ii) is equivalent to about 5 μg to about 50 μg of ethinyl estradiol.

16. The method of claim 15, wherein the daily amount of estrogen in (a)(ii) is equivalent to about 10 μg to about 30 μg of ethinyl estradiol.

17. The method of claim 16, wherein the daily amount of estrogen in (a)(ii) is equivalent to about 30 μg of ethinyl estradiol.

18. The method of claim 16, wherein the daily amount of estrogen in (a)(ii) is equivalent to about 10 μg of ethinyl estradiol.

19. The method of claim 10, wherein each cycle of the lower dose hormone regimen of (b) comprises administering to the female a regimen comprising:
(i) a combination of estrogen and progestin administered for 60 to 110 consecutive days; a combination of estrogen and progestin administered for 21 to 26 consecutive days; an estrogen-only regimen in which estrogen is administered without progestin; or a progestin-only regimen in which progestin is administered without estrogen,
wherein the daily amount of estrogen in the combination of estrogen and progestin or in the estrogen-only regimen is equivalent to about 2 μg to about 50 μg of ethinyl estradiol, and
wherein the daily amount of progestin in the combination of estrogen and progestin or in the progestin-only regimen is equivalent to about 0.01 mg to about 1.5 mg of levonorgestrel.

20. The method of claim 19, wherein each cycle of the lower dose hormone regimen further comprises:
(ii) a period of 2 to 10 consecutive days following the regimen of (b)(i) selected from the group consisting of: a hormone-free period of 2 to 10 consecutive days wherein neither estrogen nor progestin is administered to the female during the hormone-free period, and a period of 2 to 10 consecutive days wherein estrogen is administered without progestin.

21. The method of claim 19, wherein the combination of estrogen and progestin in (b)(i) is administered for 84 consecutive days.

22. The method of claim 19, wherein the combination of estrogen and progestin in (b)(i) is administered for 21 consecutive days.

23. The method of claim 19, wherein the combination of estrogen and progestin in (b)(i) is administered for 25 consecutive days.

24. The method of claim 19, wherein the daily amount of estrogen in (b)(i) is equivalent to about 10 μg to about 30 μg of ethinyl estradiol, and the daily amount of progestin in (b)(i) is equivalent to about 50 μg to about 150 μg of levonorgestrel.

25. The method of claim 24, wherein the daily amount of estrogen in (b)(i) is equivalent to about 20 μg of ethinyl estradiol, and the daily amount of progestin in (b)(i) is equivalent to about 100 μg of levonorgestrel.

26. The method of claim 20, wherein the period of 2 to 10 consecutive days in (b)(ii) is a period wherein estrogen is administered without progestin.

27. The method of claim 26, wherein the daily amount of estrogen in (b)(ii) is equivalent to about 5 μg to about 50 μg of ethinyl estradiol.

28. The method of claim 27, wherein the daily amount of estrogen in (b)(ii) is equivalent to about 10 μg to about 30 μg of ethinyl estradiol.

29. The method of claim 28, wherein the daily amount of estrogen in (b)(ii) is equivalent to about 30 μg of ethinyl estradiol.

30. The method of claim 28, wherein the daily amount of estrogen in (b)(ii) is equivalent to about 10 μg of ethinyl estradiol.

31. The method of claim 20, wherein the period in (b)(ii) is a period of 7 consecutive days.

32. The method of claim 20, wherein:
the estrogen in (a)(i) is administered for 84 consecutive days and is present in a daily amount of about 30 μg of ethinyl estradiol,
the progestin in (a)(i) is administered for 84 consecutive days and is present in a daily amount of about 150 μg of levonorgestrel,
the estrogen in (b)(i) is administered for 84 consecutive days and is present in a daily amount of about 20 μg of ethinyl estradiol,
the progestin in (b)(i) is administered for 84 consecutive days and is present in a daily amount of about 100 μg of levonorgestrel,
the periods in (a)(ii) and (b)(ii) are hormone-free periods of 7 consecutive days.

33. The method of claim 20, wherein:
the estrogen in (a)(i) is administered for 84 consecutive days and is present in a daily amount of about 30 μg of ethinyl estradiol,
the progestin in (a)(i) is administered for f 84 consecutive days and is present in a daily amount of about 150 μg of levonorgestrel,
the estrogen in (b)(i) is administered for 84 consecutive days and is present in a daily amount of about 20 μg of ethinyl estradiol,
the progestin in (b)(i) is administered for 84 consecutive days and is present in a daily amount of about 100 μg of levonorgestrel,
the period in (a)(ii) is a hormone-free period of 7 consecutive days, and
the period in (b)(ii) is a period of 7 consecutive days wherein estrogen is administered without progestin in a daily amount of about 10 μg of ethinyl estradiol.

34. The method of claim 20, wherein:
the estrogen in (a)(i) is administered for 84 consecutive days and is present in a daily amount of about 30 μg of ethinyl estradiol,
the progestin in (a)(i) is administered for 84 consecutive days and is present in a daily amount of about 150 μg of levonorgestrel,
the estrogen in (b)(i) is administered for 84 consecutive days and is present in a daily amount of about 20 μg of ethinyl estradiol,
the progestin in (b)(i) is administered for 84 consecutive days and is present in a daily amount of about 100 μg of levonorgestrel,
the periods in (a)(ii) and (b)(ii) are periods of 7 consecutive days wherein estrogen is administered without progestin in a daily amount of about 10 μg of ethinyl estradiol.

35. The method of claim 20, wherein:
the estrogen in (a)(i) is administered for 84 consecutive days and is present in a daily amount of about 30 μg of ethinyl estradiol,
the progestin in (a)(i) is administered for 84 consecutive days and is present in a daily amount of about 150 μg of levonorgestrel,
the estrogen in (b)(i) is administered for 84 consecutive days and is present in a daily amount of about 20 μg of ethinyl estradiol,
the progestin in (b)(i) is administered for 84 consecutive days and is present in a daily amount of about 100 μg of levonorgestrel,
the period in (a)(ii) is a period of 7 consecutive days wherein estrogen is administered without progestin in a daily amount of about 10 μg of ethinyl estradiol, and
the period in (b)(ii) is a hormone-free period of 7 consecutive days.

36. A method of reducing unscheduled bleeding or spotting during administration of a hormone regimen in a female, the method comprising:
(a) administering to the female one or more cycles of a run-in regimen,
wherein each cycle of the regimen comprises administering to the female a combination of estrogen and progestin for 21 to 26 consecutive days,
wherein the daily amount of estrogen administered for 21 to 26 consecutive days is equivalent to about 5 μg to about 50 μg of ethinyl estradiol,
wherein the daily amount of progestin administered for 21 to 26 consecutive days is equivalent to about 0.05 mg to about 1.5 mg of levonorgestrel,
wherein the one or more cycles of (a) is followed by
(b) administering to the female one or more cycles of a lower dose hormone regimen,
wherein the daily amount of hormone administered during any period of a cycle comprising administration of hormone is selected from the group consisting of: a daily amount of estrogen that is lower than the daily amount of estrogen administered for 21 to 26 consecutive days in (a); a daily amount of for 21 to 26 consecutive days in (a); and combinations thereof,
wherein the daily amounts are contraceptive or therapeutic amounts,
wherein unscheduled bleeding or spotting during administration of the lower dose hormone regimen of (b) is reduced following prior administration of the first hormone regimen of (a).

37. A kit comprising:
(a) 60 to 110 tablets for oral administration comprising a combination of estrogen and progestin, wherein:
(1) the estrogen in each of the tablets is present in an amount equivalent to about 5 μg to about 50 μg of ethinyl estradiol; and
(2) the progestin in each of the tablets is present in an amount equivalent to about 0.05 mg to about 1.5 mg of levonorgestrel;
(b) 2 to 10 tablets for oral administration selected from the group consisting of:
(1) 2 to 10 tablets for oral administration consisting essentially of estrogen, wherein the estrogen in each of the tablets is present in an amount equivalent to about 5 μg to about 50 μg ethinyl estradiol; and
(2) 2 to 10 tablets for oral administration consisting of a hormone-free placebo;
wherein the tablets in (a) and (b) are arranged in a fixed sequence that corresponds to the stages of daily administration; and
(c) instructions for administering the tablets of (a) and (b),
wherein the instructions require administration of the tablets of (a) and (b) prior to a lower dose hormone regimen for oral administration, wherein the daily amount of hormone administered during any period of a cycle comprising administration of hormone is selected from the group consisting of: a daily amount of estrogen that is lower than the daily amount of estrogen administered in the 60 to 110 tablets in (a); a daily amount of progestin that is lower than the daily amount of progestin administered in the 60 to 110 tablets in (a); and combinations thereof.

38. A kit comprising:
(a) 21 to 26 tablets for oral administration comprising a combination of estrogen and progestin, wherein:
(1) the estrogen in each of the tablets is present in an amount equivalent to about 5 μg to about 50 μg of ethinyl estradiol; and
(2) the progestin in each of the tablets is present in an amount equivalent to about 0.05 mg to about 1.5 mg of levonorgestrel;
(b) 2 to 10 tablets for oral administration selected from the group consisting of:
(1) 2 to 10 tablets for oral administration consisting essentially of estrogen, wherein the estrogen in each of the tablets is present in an amount equivalent to about 5 μg to about 50 μg ethinyl estradiol; and
(2) 2 to 10 tablets for oral administration consisting of a hormone-free placebo;
wherein the tablets in (a) and (b) are arranged in a fixed sequence that corresponds to the stages of daily administration; and
(c) instructions for administering the tablets of (a) and (b),
wherein the instructions require administration of the tablets of (a) and (b) prior to a lower dose hormone regimen for oral administration, wherein the daily amount of hormone administered during any period of a cycle comprising administration of hormone is selected from the group consisting of: a daily amount of estrogen that is lower than the daily amount of estrogen administered in the 21 to 26 tablets in (a); a daily amount of progestin that is lower than the daily amount of progestin administered in the 21 to 26 tablets in (a); and combinations thereof.
